SEQUENCE LISTING (36 sequences)

SEQ ID NO: 1 - protein, 556 aa, Mycoplasma genitalium
MQFKKHKNSVKFKRKLFWTIGVLGAGALTTFSAVMITNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 2 - protein, 537 aa, Artificial Sequence
  Synthetic polypeptide
HHHHHHSSGLVPRGSHMTNLVNQSGYALVASGRSGNLGFKLFSTQSPSAEVKLKSLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVIYDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDENIKRITASGKYSVQFQKLENDTDSSLERMTKAVEGLVTVIGEEKFETVDITGVSSDTNEVKSLAKELKTNALGVKLKL

SEQ ID NO: 3 - protein, 406 aa, Mycoplasma genitalium
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 4 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 5 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDQYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 6 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELDLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 7 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 8 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLIKIVNTNPDVDDDIVYRSLKELNLHLEEAYREGDNIYYRVNENYYPGASIYENERASRDSEFQNEILKRAEQNGVTFDEN

SEQ ID NO: 9 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAIGDIYNYRRFERQFQGYFAGGYIDKYLIKNVNTNKDSDDDLVYRSLKELNLHLEEAYREGDNTYYRVNENYYPGASIYENERASRDSEFQNEILKRAEQNGVTFDEN

SEQ ID NO: 10 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKLVNTNPDVDDDIVYRSLKELNLHLEEAYREGDNTYYRVNENYYPGASIYENERASRDSEFQNEILKRAEQNGVTFDEN

SEQ ID NO: 11 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 12 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELDLPESVKKVSLYGDYTGVNVAKQIFANVVELEFYSTSKANSFGFNPLVLGSKTNVINDLFVSKPFTHIDLTQVTLQNSDNSAIDANKLKQAIGDIYNYRRFERQFQGYFAGGYIDKYLIKIVNTNPDVDDDIVYRSLKELNLHLEEAYREGDNIYYRVNENYYPGASIYENERASRDSEFQNEILKRAEQNGVTFDEN

SEQ ID NO: 13 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 14 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 15 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAIGDIYNYRRFERQFQGYFAGGYIDKYLIKIVNTNPDVDDDIVYRSLKELNLHLEEAYREGDNIYYRVNENYYPGASIYENERASRDSEFQNEILKRAEQNGVTFDEN

SEQ ID NO: 16 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKPTTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 17 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVDLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 18 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVPLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 19 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAIGDIYNYRRFERQFQGYFAGGYIDKYLIKNVNTNKDSDDDLVYRSLKELNLHLEEAYREGDNTYYRVNENYYPGASIYENERASRDSEFQNEILKRAEQNGVTFDEN

SEQ ID NO: 20 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAIGDIYNYRRFERQFQGYFAGGYIDKYLIKNVNTNKDSDDDLVYRSLKELNLHLEEAYREGDNTYYRVNENYYPGASIYENERASRDSEFQNEILKRAEQNGVTFDEN

SEQ ID NO: 21 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVASDGSLLSYYQSTEYFNNRVLMPILQTTNGTLMANNRGYDDVFRQVPSFSGWCNTKATTVSTSNNLTYDKWTYFACKGSPLYDSYPNHFFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFASKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 22 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWCNTKATTVSTSNNLTYDKWTYFACKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 23 - protein, 582 aa, Mycoplasma pneumoniae
MKLNFKIKDKKTLKRLKKGGFWALGLFGAAINAFSAVLIVNEVLRLQSGETLIASGRSGNLSFQLYSKVNQNAKSKLNSISLTDGGYRSEIDLGDGSNFREDFRNFANNLSEAITDAPKDLLRPVPKVEVSGLIKTSSTFITPNFKAGYYDQVAADGKTLKYYQSTEYFNNRVVMPILQTTNGTLTANNRAYDDIFVDQGVPKFPGWFHDVDKAYYAGSNGQSEYLFKEWNYYVANGSPLYNVYPNHHFKQIKTIAFDAPRIKQGNTDGINLNLKQRNPDYVIINGLTGDGSTLKDLELPESVKKVSIYGDYHSINVAKQIFKNVLELEFYSTNQDNNFGFNPLVLGDHTNIIYDLFASKPFNYIDLTSLELKDNQDNIDASKLKRAVSDIYIRRRFERQMQGYWAGGYIDRYLVKNTNEKNVNKDNDTVYAALKDINLHLEETYTHGGNTMYRVNENYYPGASAYEAERATRDSEFQKEIVQRAELIGVVFEYGVKNLRPGLKYTVKFESPQEQVALKSTDKFQPVIGSVTDMSKSVTDLIGVLRDNAEILNITNVSKDETVVAELKEKLDRENVFQEIRT

SEQ ID NO: 24 - protein, 415 aa, Mycoplasma pneumoniae
SISLTDGGYRSEIDLGDGSNFREDFRNFANNLSEAITDAPKDLLRPVPKVEVSGLIKTSSTFITPNFKAGYYDQVAADGKTLKYYQSTEYFNNRVVMPILQTTNGTLTANNRAYDDIFVDQGVPKFPGWFHDVDKAYYAGSNGQSEYLFKEWNYYVANGSPLYNVYPNHHFKQIKTIAFDAPRIKQGNTDGINLNLKQRNPDYVIINGLTGDGSTLKDLELPESVKKVSIYGDYHSINVAKQIFKNVLELEFYSTNQDNNFGFNPLVLGDHTNIIYDLFASKPFNYIDLTSLELKDNQDNIDASKLKRAVSDIYIRRRFERQMQGYWAGGYIDRYLVKNTNEKNVNKDNDTVYAALKDINLHLEETYTHGGNTMYRVNENYYPGASAYEAERATRDSEFQKEIVQRAELIGVVFE

SEQ ID NO: 25 - DNA, 1287 bp, Artificial Sequence
  Synthetic polynucleotide
atgggcagcagccatcaccatcatcatcacagcagcggtctggtgccgcgtggtagccacatgagcctgagcctgaacgatggtagctaccagagcgagatcgacctgagcggcggtgccaacttccgtgaaaaattccgcaactttgctaacgagctgagcgaagccattaccaatagcccaaagggcctggatcgtccagtgccgaaaaccgagatcagcggcctgattaagaccggtgacaactttatcaccccgagcttcaaggcgggctactatgatcacgtggctgaggacggtagcctgctgagctactatcagagcaccgagtactttaacaaccgtgtgctgatgccgattctgcagaccaccaacggcaccctgatggccaacaaccgtggttatgacgacgtgttccgtcaggtgccgcgtttcccgggctggagcaacaccaaagcgaccaccgtgagcaccagcaacaacctgacctacgacaagtggacctatttcgccgcgaaaggtagcccgctgtacgatcagtatccgaaccacacctttgaggacgtgaaaaccctggctatcgatgccaaggacattagcgcgctgaaaaccaccatcgatagcgaaaagccgacctacctgatcattcgcggtctgagcggcaacggtagccagctgaacgagctgcagctgccggaaagcgtgaagaaagtgagcctgtacggcgactataccggtgtgaacgtggccaaacagatttttgcgaacgtggtggagctggaattctatagcaccagcaaggcgaacagcttcggctttaacccgctggtgctgggtagcaaaaccaacgtgatcaacgacctgttcgtgagcaagccgttcacccacattgacctgacccaggtgaccctgcagaacagcgataacagcgcgatcgacgctaacaagctgaaacaggctgtgggcgatatctacaactatcgtcgcttcgagcgtcagtttcagggttacttcgccggcggttacatcgataagtatctggtgaaaaacgtgaacaccaacaaagacagcgacgatgacctggtgtaccgcagcctgaaggaactgaacctgcacctggaggaagcttatcgtgagggcgacaacacctactatcgcgtgaacgaaaactactatccgggtgccagcatctacgagaacgaacgtgcgagccgcgatagcgagtttcagaacgaaattctgaagcgtgcggagcagaacggcgtgaccttcgacgaaaactaataa

SEQ ID NO: 26 - DNA, 1227 bp, Artificial Sequence
  Synthetic polynucleotide
atgagcctgagcctgaacgatggcagctaccagagcgagatcgacctgtctggcggagccaacttcagagagaagttcagaaacttcgccaacgagctgagcgaggccatcacaaacagccccaaaggcctggacagacccgtgcctaagacagagatcagcggcctgatcaagaccggcgacaacttcatcacccctagcttcaaggccggctactacgatcacgtggcctctgatggcagcctgctgagctactaccagtccaccgagtacttcaacaaccgggtgctgatgcccatcctccagaccaccaatggcaccctgatggccaacaacagaggctacgacgacgtgttcagacaggtgcccagctttagcggctggtccaataccaaggccaccaccgtgtccaccagcaacaacctgacctacgacaagtggacctacttcgccgccaagggcagccctctgtacgacagctaccccaaccacttcttcgaggacgtgaaaaccctggccatcgacgccaaggatatcagcgccctgaaaaccaccatcgacagcgagaagcccacctacctgatcatcagaggactgagcggcaacggcagccagctgaatgaactccagctgcctgagagcgtgaagaaggtgtccctgtacggcgattacaccggcgtgaacgtggccaagcagatcttcgccaatgtggtggaactggaattctacagcaccagcaaggccaacagcttcggcttcaaccctctggtgctgggcagcaagaccaacgtgatcaacgacctgttcgccagcaagcccttcacacacatcgatctgacccaagtgaccctccagaacagcgacaacagcgccattgatgccaacaagctgaaacaggccgtgggcgacatctacaactacagaagattcgagcggcagttccagggctacttcgctggcggctacatcgacaagtacctggtcaagaacgtgaacaccaacaaggacagcgacgacgacctggtgtacagaagcctgaaagagctgaacctgcacctggaagaggcctacagagagggcgacaacacctactacagagtgaacgagaactactacccaggcgccagcatctacgagaacgagagagccagcagagacagcgagttccagaacgagatcctgaagcgggccgagcagaatggcgtgaccttcgacgagaactgatga

SEQ ID NO: 27 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYEAGGYIDKYLVKNVNTNKDSDDDLVYRSLKELNLHLEEAYREGDNTYYRVNENYKPGASIYENERASRDSEFQNEILKRAEQNGVTFDEN

SEQ ID NO: 28 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWCNTKPTTVSTSNNLTYDKWTYFACKGSPLYDQYPNHTFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFVSKPFTHIDLTQVPLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 29 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFPGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 30 - protein, 406 aa, Artificial Sequence
  Synthetic polypeptide
SISLTDGGYRSEIDLGDGSNFREDFRNFANNLSEAITDAPKDLLRPVPKVEVSGLIKTSSTFITPNFKAGYYDQVAEDGKTLKYYQSTEYFNNRVVMPILQTTNGTLTANNRAYDDIFVDQGVPRFPGWFHDVDKAYYAGSNGQSEYLFKEWNYYVANGSPLYNQYPNHTFKQIKTIAFDAPRIKQGNTDGINLNLKQRNPDYVIINGLTGDGSTLKDLELPESVKKVSIYGDYHSINVAKQIFKNVLELEFYSTNQDNNFGFNPLVLGDHTNIIYDLFVSKPFNYIDLTSLELKDNQDNIDASKLKRAVSDIYIRRRFERQMQGYWAGGYIDRYLVKNTNEKNVNKDNDTVYAALKDINLHLEETYTHGGNTMYRVNENYYPGASAYEAERATRDSEFQKEIVQR

SEQ ID NO: 31 - protein, 677 aa, Artificial Sequence
  Synthetic polypeptide
MYRMQLLSCIALSLALVTNSAPLEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGAGGGSGGGSGGGSGGGMSLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTDGTLMANNRGYDDVFRQVPRFPGWSNTKATTVSTSNNLYYDKWTYFAAKGSPLYDQYPNHTFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 32 - protein, 673 aa, Artificial Sequence
  Synthetic polypeptide
MYRMQLLSCIALSLALVTNSAPLERKSSVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAGGGSGGGSGGGSGGGMSLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTDGTLMANNRGYDDVFRQVPRFPGWSNTKATTVSTSNNLYYDKWTYFAAKGSPLYDQYPNHTFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 33 - protein, 471 aa, Artificial Sequence
  Synthetic polypeptide
MKFNKEQQNAFYEILHLPNLNEEQRNAFIQSLKDDPSQSANLLAEAKKLNDAQAPGGSGGSGGSGSLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 34 - protein, 406 aa, Artificial Sequence
  Synthetic polypeptide
SLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKPTTVSTSNNLTYDKWTYFAAKGSPLYDQYPNHTFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFVSKPFTHIDLTQVPLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 35 - protein, 469 aa, Artificial Sequence
  Synthetic polypeptide
MEEVTIKANLILPGCCTQTAEFKGTFEKATSEAYAYADTLKKDNGEWTVDVADKGYTLNIKFAGGSGGSGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKATTVSTSNNLTYDKWTYFAAKGSPLYDQYPNHTFEDVKTLAIDAKDISALKTTIDSEKPTYLIIRGLSGNGSQLNELQLPESVKKVSLYGDYTGVNVAKQIFANVVELEFYSTSKANSFGFNPLVLGSKTNVINDLFVSKPFTHIDLTQVTLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

SEQ ID NO: 36 - protein, 458 aa, Artificial Sequence
  Synthetic polypeptide
GGCGGMKQLEDKIEELLSKIYHLENEIARLKKLIGEGGGSGGGSGGGSGGGMSLSLNDGSYQSEIDLSGGANFREKFRNFANELSEAITNSPKGLDRPVPKTEISGLIKTGDNFITPSFKAGYYDHVAEDGSLLSYYQSTEYFNNRVLMPILQTTNGTLMANNRGYDDVFRQVPRFPGWSNTKPTTVSTSNNLTYDKWTYFAAKGSPLYDQYPNHTFEDVKTLAIDAKDISALKTTIDSEKPTYLIIRGLSGDGSQLNELQLPESVKKVSLYGDYTGVNVAKQIFANVVELEFYSTSKANSFGFNPLVLGSKTNVINDLFVSKPFTHIDLTQVPLQNSDNSAIDANKLKQAVGDIYNYRRFERQFQGYFAGGYIDKYLVKNVNTNKDSDDDLVYRSLKELNLHLEEAYREGDNTYYRVNENYYPGASIYENERASRDSEFQNEILKRAEQNGVTFDEN

(19) United States
(12) Patent Application Publication
(10) Pub. No.: US 2024/0309055 A1
(43) Pub. Date: Sep. 19, 2024

(54) PROTEIN M ANALOGS AND FUSION PROTEINS AND THEIR USE FOR INHIBITING ANTIBODY FUNCTION

(71) Applicant: The University of North Carolina at Chapel Hill, Chapel Hill, NC (US)

(72) Inventors: Charles Askew, Durham, NC (US); Chengwen Li, Chapel Hill, NC (US); Brian Kuhlman, Chapel Hill, NC (US); David Forrest Thieker, Durham, NC (US)

(21) Appl. No.: 18/264,136

(22) PCT Filed: Feb. 3, 2022
(86) PCT No.: PCT/US2022/015127

§ 371 Date: Aug. 3, 2023

Related U.S. Application Data
(60) Provisional application No. 63/145,188, filed on Feb. 3, 2021.

(51) Int. Cl. C07K 14/30; A61K 38/00; A61P 37/00; C12N 15/86

(52) U.S. Cl. CPC C07K 14/30; A61P 37/00; C12N 15/86; A61K 38/00; C07K 2319/00; C12N 2750/14143

(57) ABSTRACT

This invention relates to methods and compositions for binding antibodies. The methods may be used to isolate antibodies, treat disorders related to excess antibodies, acutely block antibodies to stop an autoimmune or inflammatory response, and inhibit neutralizing antibodies. In embodiments, the invention relates to methods of inhibiting neutralizing antibodies against a heterologous agent when the heterologous agent is administered to a subject, comprising administering to the subject an effective amount of Mycoplasma protein M or a functional fragment or derivative thereof, thereby inhibiting neutralization of the heterologous agent. The invention further relates to modified Mycoplasma protein M or functional fragments thereof having increased thermostability relative to wild-type protein M and their use in the methods of the invention.

### STATEMENT OF PRIORITY

This application claims the benefit of U.S. Provisional Application Ser. No. 63/145,188, filed Feb. 3, 2021, the entire contents of which are incorporated by reference herein.

### FIELD OF THE INVENTION

This invention relates to methods and compositions for binding antibodies. The methods may be used to isolate antibodies, treat disorders related to excess antibodies, acutely block antibodies to stop an autoimmune or inflammatory response, and inhibit neutralizing antibodies. In embodiments, the invention relates to methods of inhibiting neutralizing antibodies against a heterologous agent when the heterologous agent is administered to a subject, comprising administering to the subject an effective amount of Mycoplasma protein M or a functional fragment or derivative thereof, thereby inhibiting neutralization of the heterologous agent. The invention further relates to modified Mycoplasma protein M or functional fragments thereof having increased thermostability relative to wild-type protein M and their use in the methods of the invention.

### BACKGROUND OF THE INVENTION

Approximately 1 in 10 people in the US suffers from a rare genetic disease, which can seriously impact life-span, quality of life, independence, and economic potential. Gene therapy is the most promising form of treatment for the correction of heritable diseases. Among gene therapy delivery vehicles, adeno-associated virus (AAV) vectors have showed therapeutic effect in numerous clinical trials. The first FDA approved gene therapy has been used in patients with blindness. The number of clinical trials for AAV gene therapy has grown substantially due to its safety and success in targeting many different types of organs for long-term therapeutic gene expression. Despite clinical success, the major barrier to AAV mediated gene delivery is the high prevalence of neutralizing antibodies (NAbs), which block vector transduction of target tissues. Greater than 90% of the general population has been exposed to AAV through natural infection, and over 50% of people are serum positive for NAbs against AAV. Identification of pre-existing NAbs above a specific threshold during clinical trial screening disqualifies patients from enrollment, since NAbs severely attenuate therapeutic effectiveness and cause outcome variability.

To overcome AAV NAbs, several approaches have been employed: serial plasmapheresis, immune suppressive drugs, and alteration of the vector capsid to eliminate immune epitopes. Plasmapheresis is inefficient, requiring multiple rounds that remove 2-3 fold of the remaining NAbs each session and is only capable of addressing low titer of NAbs. Plasmapheresis is also time consuming and puts patients at risk for communication of nosocomial infections through simultaneous depletion of antibodies and repeated intravenous needle exposure. Steroids or pharmacological immune suppression to kill B-cells carries significant health risks and requires prolonged regimens to reduce NAbs by even 10-fold. AAV capsid engineering is an innovative method but ultimately inadequate against polyclonal anti-AAV sera, where a modest 10-fold increase in antibody escape is observed in vivo. Additionally, modification of the capsid structure to alter NAb recognition epitopes typically results in less potent vectors and defective vector production because those altered surface regions are multifunctional. None of the current approaches successfully overcome pre-existing anti-AAV NAbs above the typical thresholds set for clinical trial exclusion, or allow repeat administration of the same AAV vectors.

Mycoplasma protein M has been identified as capable of non-specifically binding antibodies and blocking their ability to bind antigen (U.S. Pat. No. 9,593,150; US Publication No, 2017/0320921).

The present invention overcomes shortcomings in the art by providing a vector independent protein based method to universally block Nabs and other methods and compositions based on antibody binding.

### SUMMARY OF THE INVENTION

The present invention is based, in part, on the development of a vector independent protein based method to universally block NAbs and demonstration that this method is effective against a broad range of pre-existing NAb concentrations. The invention pioneers the use of a unique Mycoplasma-derived protein and its analogues, termed protein M, to enable successful gene delivery by preventing neutralization of heterologous agents (e.g., AAV vectors) by NAbs upon administration of the heterologous agent to a subject. Protein M was shown to block mammalian IgG, IgM, and IgA antibody classes in a species and antigen independent manner by universally binding to conserved regions on the antibody light and heavy chains, causing structural interference with the antigen recognizing CDR regions. Protein M binds to antibodies with nanomolar affinity, and prevents antigen-antibody union for a variety of different tested immunoglobulin/antigen pairs. The inventors discovered that protein M blocks antibody recognition of AAV and prevents antibody-mediated neutralization of AAV. It has been demonstrated that the level of AAV vector escape from NAbs is proportional to the NAb titer and volume, amount of protein M, and amount of AAV. Protein M mediated NAb escape has been validated in vitro and in vivo using human serum (IVIG) and serum from AAV immunized mice. It was demonstrated that protein M can be administered alone prior to AAV administration, or formulated with AAV for NAb evasion. The effectiveness of this approach depends on the interaction of protein M with immunoglobulin before AAV is neutralized. This approach can be used to overcome NAbs to multiple heterologous agents (e.g., AAV vector serotypes) while maintaining the unique or beneficial properties of each agent for specific gene therapy applications.

The invention further relates to the use of protein M in other methods in which binding of antibodies is beneficial, including treatment of autoimmune disorders, treatment of disorders associated with excess antibodies, methods of isolating antibodies, and methods of performing immunoassays.

The invention further relates to modified protein M proteins having increased thermostability and/or other advantageous characteristics (e.g., increased or maintained pH stability relative to wild-type Mycoplasma protein M or a functional fragment thereof) for carrying out the methods of the invention in vivo or at elevated temperatures.

Thus, one aspect of the invention relates to a modified Mycoplasma protein M or a functional fragment thereof, having one or more amino acid mutations that increase or maintain thermostability and/or pH stability of the Mycoplasma protein M or a functional fragment thereof relative to wild-type Mycoplasma protein M or a functional fragment thereof. In some embodiments, the thermostability is maintained up to 75° C., 70° C., 65° C., 60° C., 55° C., 50° C., 45° C., 40° C., or 38° C. and/or the pH stability is maintained from a pH of 2 to 8, from a pH of 2.5 to 7.5, or from a pH of 4.5 to 7.5.

Another aspect of the invention relates to a fusion protein comprising a modified Mycoplasma protein M or a functional fragment thereof, a linker, and a peptide.

An additional aspect of the invention relates to a polynucleotide encoding the modified Mycoplasma protein M or a functional fragment thereof of the invention and a vector or transformed cell comprising the same.

Another aspect of the invention relates to a method of inhibiting neutralization of a heterologous agent by neutralizing antibodies upon administration of the heterologous agent to a subject, comprising administering to the subject an effective amount of Mycoplasma protein M or a functional fragment or derivative thereof, thereby inhibiting neutralization of the heterologous agent.

A further aspect of the invention relates to a method of expressing a polypeptide or functional nucleic acid in a subject, comprising administering to the subject (a) a nucleic acid delivery vector encoding the polypeptide or functional nucleic acid, and (b) an effective amount of Mycoplasma protein M or a functional fragment or derivative thereof, thereby expressing the polypeptide or functional nucleic acid in the subject.

An additional aspect of the invention relates to a method of treating a disorder in a subject in need thereof, wherein the disorder is treatable by expressing a polypeptide or functional nucleic acid in the subject, comprising administering to the subject (a) a therapeutically effective amount of a nucleic acid delivery vector encoding the polypeptide or functional nucleic acid, and (b) an effective amount of Mycoplasma protein M or a functional fragment or derivative thereof, thereby treating the disorder in the subject.

Another aspect of the invention relates to a method of editing a gene in a subject, comprising administering to the subject (a) a gene editing complex, and (b) an effective amount of Mycoplasma protein M or a functional fragment or derivative thereof, thereby expressing the polypeptide or functional nucleic acid in the subject.

An additional aspect of the invention relates to a method of treating an autoimmune disease in a subject in need thereof, comprising administering to the subject an effective amount of Mycoplasma protein M or a functional fragment or derivative thereof, thereby treating the autoimmune disease.

A further aspect of the invention relates to a method of treating a disorder associated with excess antibodies in a subject in need thereof, comprising administering to the subject a therapeutically effective amount of Mycoplasma protein M or a functional fragment or derivative thereof, thereby treating the disorder associated with excess antibodies. Another aspect of the invention relates to a method of treating and/or preventing acute transplant rejection of solid organs associated with recognition of the transplant by recipient antibodies in a subject in need thereof, comprising administering to the subject prior to and/or after a transplant, a therapeutically effective amount of the Mycoplasma protein M or a functional fragment or derivative thereof.

Another aspect of the invention relates to a method of isolating a compound comprising an antibody light chain variable region and/or heavy chain variable region from a sample, the method comprising contacting the compound with the modified Mycoplasma protein M or a functional fragment thereof of the invention attached to a solid support, then eluting the compound from the modified Mycoplasma protein M or a functional fragment thereof.

An additional aspect of the invention relates to a method of performing an immunoassay, the method comprising using the modified Mycoplasma protein M or a functional fragment thereof of the invention to bind a compound comprising an antibody light chain variable region and/or heavy chain variable region.

A further aspect of the invention relates to a kit comprising the modified Mycoplasma protein M or a functional fragment thereof of the invention.

These and other aspects of the invention are set forth in more detail in the description of the invention below.

### BRIEF DESCRIPTION OF THE DRAWINGS

FIG. 1 shows human IVIG contains AAV neutralizing antibodies that dose-dependently inhibit AAV transduction. A 2-fold dilution series of human IVIG was used to generate an AAV2 neutralization plot. Beginning with 50 μg, IVIG was diluted stepwise to less than 1 μg, and each dilution was incubated for 1 h at 4° C. with 2×10⁸ viral genomes of AAV2-Luciferase before seeding the wells. The luminescent reporter signal generated by the AAV2 transgene expression is a functional read-out of AAV transduction and is proportional to the amount of AAV that was available to transduce cultured HEK-293 cells. In this experiment, each well of a 48-well plate was seeded with 1×10⁵ cells and transduced at an MOI of 2,000 in serum free media (n=3 technical replicates per condition). Measurement of luciferase activity was performed at 48 h post-transduction on a plate reader after cell lysis and addition of luciferin. The relationship between the percent of AAV neutralized for a given IVIG quantity was determined at 12.5 μg of IVIG to be 72% (+/−1.3%) neutralization compared to the no-IVIG control that contained an equivalent volume of phosphate buffered saline (PBS). Furthermore, 25 μg of IVIG neutralized 96% (+/−0.2%) while 50 μg of IVIG neutralized 99% (+/−0.17%) of AAV2.

FIG. 2 shows protein M protects AAV from neutralizing antibodies present in human IVIG. Using 12.5 μg IVIG which was established in the previous figure to neutralize ˜70% of AAV2 at a dose of 2×10⁸ vg, escape from antibody mediated neutralization was investigated using a 2-fold dilution series of protein M. In this case, the experiment began with a molar ratio of 8 protein M molecules to 1 IgG molecule, which is a 4-fold excess of protein M given that 1 IgG molecule has 2 protein M binding sites that must be occupied for complete antibody inactivation. In each well, IVIG was first incubated with individual protein M dilutions for 1 h at 4° C., followed by addition of AAV2-Luciferase (2×10⁸ vg) and incubation for an additional 1 h at 4° C. HEK-293 cells (1×10⁵) were then added to the wells and incubated for 48 h before measuring the luciferase reporter signal. Wells that contained IVIG but no protein M demonstrated a ˜68% (+/−3.3%) reduction in AAV signal as compared to no-IVIG control wells containing phosphate buffered saline (PBS). However, protein M dose dependently blocked neutralization of AAV at molar ratios greater than 2:1, which completely prevented neutralization (108%-193% of no-IVIG control) while ratios of 1:1 or 0.5:1 only partially blocked neutralization (52% and 40% of no-IVIG control respectively). Additionally, at ratios of 8:1 and 4:1 a dose dependent increase of AAV luciferase signal over the no-IVIG control was observed. All wells were done in triplicate in serum free media (n=3 technical replicates per condition) in a 48-well plate and luciferase signal was measured 48 h post-transduction.

FIG. 3 shows excess protein M provides little additional protection from NAbs at molar ratios greater than 8:1 and protein M enhances AAV transduction. In an effort to establish the upper limit of protein M molar ratios that protect or enhance transduction of AAV, with or without neutralizing antibody conditions, the NAb escape assay was repeated with molar ratios of 8:1 or 20:1. Addition of 12.5 μg of IVIG neutralized 80% (+/−2.6%) of the AAV2-Luciferase signal compared with the no-IVIG PBS control wells. Similar to the previous experiment, protein M pre-incubated with 12.5 μg IVIG (8:1 molar ratio, 33 μg protein M) for 1 h at 4° C. followed by 1 h at 4° C. incubation with AAV prevented AAV neutralization and enhanced luciferase signal to 194% (+/−24%) of the no-IVIG control with PBS. At 10-times the amount of protein M necessary for NAb escape (20:1 molar ratio, 75 μg protein M), a modest increase in luciferase activity was observed to 256% (+/−44%) over the no-IVIG control containing PBS. The luciferase signal of the 20:1 ratio was not statistically different from the 8:1 ratio in the presence of IVIG. Furthermore, without IVIG when either 75 μg or 33 μg of protein M were incubated alone with AAV2-Luciferase for 1 h at 4° C., there was no significant difference in the amount of luciferase signal (208% vs 216% respectively), confirming that the increase in luciferase signal relative to the PBS control is due to protein M based enhancement. Each well was seeded with 1×10⁵ HEK-293 cells in serum free media and transduced at an MOI of 2,000 (n=3 technical replicates per well condition) in a 48-well plate format.

FIG. 4 shows protein M enhancement of AAV transduction is dose dependent. A 2-fold dilution series of protein M, beginning with 33 μg, demonstrates that incubation of AAV2-Luciferase with protein M results in an increase in luciferase signal relative to the PBS+AAV control. Enhancement of the luciferase signal is abolished at concentrations of protein M below 2 μg for 2×10⁸ viral genomes, or a molar ratio of 40,000 protein M molecules to 1 genome containing AAV2-Luciferase particle. Enhancement begins to saturate at about 33 μg of protein M for 2×10⁸ vector particles, or roughly 700,000 molecules of protein M to 1 genome containing AAV2-Luciferase particle. Protein M dilutions were incubated with AAV for 1 h at 4° C. prior to transduction. Each well was seeded with 1×10⁵ HEK-293 cells in serum free media and transduced at an MOI of 2,000 (n=3 technical replicates per well condition) in a 48-well plate format.

FIG. 5 shows protein M based enhancement of AAV transduction is dependent on protein M direct interaction with the AAV capsid. Three different conditions were tested with AAV2-Luceiferase: protein M pre-incubation with AAV for 1 h prior to transduction (−1 h), addition of protein M to AAV at the pen-transduction time point (0 h), or addition of protein M to the wells 18 h post-transduction by AAV (18 h). Two negative control conditions without protein M were used: AAV was incubated alone for 1 h at 4° C. in cell culture media prior to cell seeding and transduction (representative of conditions in the pre-incubation and pen-transduction groups), or AAV was diluted in PBS and added to cell culture at the time of cell seeding and transduction (representative of the conditions in the post-transduction group). At 18 h post-transduction, an additional volume of PBS was added to the PBS control group to mimic conditions in which an equivalent volume of protein M was added to the post-transduction group. No enhancement of AAV luciferase signal was seen in the wells where protein M was added peri-transduction or post-transduction, while dose dependent enhancement was observed in the pre-incubation group, indicating protein M interaction with AAV was necessary for the enhancement mechanism. All wells were seeded with 1×10⁵ Huh7 cells and each well was transduced by 2×10⁸ vg of AAV, n=3 technical replicates per well condition in a 48-well plate format.

FIG. 6 shows protein M does not prevent AAV neutralization after NAbs have bound the capsid. AAV2-Luciferase was first incubated with different dilutions of IVIG at 4° C. for 1 h, then protein M was added and incubated for an additional 1 h at 4° C. A double negative control group received AAV only, while a positive control received 33 μg of protein M incubated with AAV for 1 h at 4° C. before transduction. Three different dilutions of IVIG were used that contained either 200 μg, 50 μg, or 12.5 μg. Compared to the protein M negative control groups that contained only AAV incubated with dilutions of IVIG, 33 μg of protein M does not enable post-neutralization enhancement or escape from neutralizing antibodies when 99% of AAV has already been neutralized by either 50 μg or 200 μg of IVIG. However, when 12.5 μg of IVIG is incubated with AAV about 30% of the vector remains un-neutralized (see FIGS. 1, 2, and 3), and 33 μg of protein M was able to enhance transduction of this fraction post-neutralization compared to the protein M negative control wells containing AAV and 12.5 μg IVIG. Unlike previous figures, luciferase measurements were taken at only 24 h post transduction, which could explain why luciferase signal from the group with 12.5 μg IVIG plus AAV is less than 30% of the double negative control. Each well was seeded with 1×10⁵ HEK-293 cells in serum free media and transduced at an MOI of 2,000 (n=3 technical replicates per well condition) in a 48-well plate format.

FIG. 7 shows pre-incubation of protein M with AAV protects the vector from later neutralization by IVIG. For this neutralization assay the quantity of protein M was kept the same (8.25 μg) while various amounts of IVIG were used to achieve different molar ratios (from 50 μg to 3.12 μg IVIG). Protein M was first incubated with AAV2-Luciferase for 1 h at 4° C., followed by addition of IVIG for 1 h at 4° C. before transducing the cell cultures. Positive control groups contained AAV plus either 33 μg, 8.25 μg, or 1 μg protein M, while the negative control group contained only AAV incubated with PBS. Protein M mediated enhancement of the non-neutralized fraction of AAV by 2-3 fold could be observed. Each well was seeded with 1×10⁵ HEK-293 cells in serum free media and transduced at an MOI of 2,000 (n=3 technical replicates per well condition) in a 48-well plate format. Luciferase measurements were taken 24 h post transduction.

FIG. 8 shows pre-incubating protein M with AAV protects the vector from neutralization by IVIG in an excess of background serum immunoglobulins. For this experiment 25 μg of IVIG was used, which was previously shown to neutralize ˜95% of AAV2-Luciferase at a dose of 2×10⁸ viral genomes. The 25 μg of Human IVIG was added to cell culture wells containing 10% Fetal Bovine Serum (FBS), with an estimated Bovine IgG concentration of 350 μg as determined by ELISA performed by the manufacturer. Another set of cell culture wells containing the same quantity of 10% FBS served as a no-IVIG control. Protein M at molar ratios of 4:1, 2:1, and 1:1 (based on Bovine IgG content) was incubated with AAV for 1 h at 4° C. before being added the no-IVIG control wells or 25 μg IVIG containing wells each with 10% FBS. AAV was incubated with PBS instead of protein M in the negative control group, and added to wells containing either 10% FBS or 10% FBS with 25 μg of IVIG. The results demonstrate that protein M pre-incubation protected AAV2-Luciferase from neutralization by IVIG even at a ratio of 1 protein M molecule to 1 Bovine IgG molecule, although this amount of protein M (108 μg) was still in a 12-fold greater ratio than Human IVIG (25 μg). Each well was seeded with 1×10⁵ HEK-293 cells and transduced at an MOI of 2,000 (n=3 technical replicates per well condition) in a 48-well plate format. Luciferase measurements were taken 24 h post transduction.

FIG. 9 shows protein M allows in vitro escape of AAV8 from neutralizing antibodies found in pooled polyclonal serum collected from AAV8 immunized mice. For this experiment 10 μl of pooled polyclonal serum from AAV8 immunized mice was serially diluted in PBS, first 10-fold to generate 1 μl and 0.1 μl serum containing wells. The 0.1 μl serum well was then further diluted in a 2-fold dilution series. AAV8-Luciferase vector (2×10⁸ viral genomes per well) was incubated with the dilutions of neutralizing serum for 1 h at 4° C. before transduction. Protein M was added to a 10-fold dilution series of the same AAV8 neutralizing mouse serum in a ratio of 2 protein M molecules to an estimated 1 immunoglobulin molecule. Beginning with 6.5 μg of protein M to an estimated 10 μg of immunoglobulin in 1 μl of neutralizing mouse serum, each of the protein M and serum samples were reciprocally diluted separately before being combined for a 1 h incubation at 4° C. All samples were then incubated for 1 h at 4° C. with AAV8-Luciferase before being added to cells. Data from the resulting neutralization experiment were normalized to no serum control wells containing only AAV8, and the luciferase signals were then fit with double exponential decay functions to estimate the curve between data points. Using the modeled curve it was found that 50% neutralization of AAV8 by the polyclonal serum occurred at a volume of 0.0039 μl for an effective titer of 1:2,564. However, 50% neutralization of the same serum incubated with a 2:1 molar ratio of protein M occurred at a serum volume of 0.2744 μl resulting in an estimated effective titer of 1:36. This result demonstrates that protein M is capable of protecting AAV over nearly a 100-fold difference in serum concentration in vitro. All wells were seeded with 5×10⁴ HEK-293 cells in a 96-well plate format (MOI of 4,000), n=3 technical replicates per well condition, and luciferase was measured 48 h post-transduction.

FIG. 10 shows in vivo administration of protein M to mice with passive immunity to AAV8 results in neutralizing antibody escape. In this experiment mice were given various volumes of polyclonal AAV8 serum (titer 1:2,564 from the previous figure) through passive transfer via IV injection, followed within 15-20 minutes by IV administration of 2×10¹⁰ viral genomes of AAV8-Luciferase. This established a neutralization curve based on serum volume delivered to each mouse. It was found that over 50% neutralization of the AAV8-Luciferase signal occurred from 1 μl to 0.001 μl of transferred serum as compared to a group of naïve mice without passive transfer of serum. However, when an estimated 2:1 ratio of protein M (6.3 mg) was delivered to mice after passive transfer but before AAV administration, then complete escape from neutralizing antibodies was achieved at transferred serum volumes of 0.3 μl and 1 μl indicated protein M mediated escape from neutralizing antibodies of a 1,000-fold change in NAb concentration. Furthermore, escape from neutralization was dose dependent for the 0.3 μl passively transferred serum group, demonstrating NAb escape is decreased for 1:1 (3.15 mg) and 0.5:1 (1.58 mg) estimated molar ratios compared to the no-serum control group. For all groups n=5 mice per group condition and luciferase signal from the liver was measured at 24 h post AAV administration.

FIG. 11 shows the averaged raw luminescence quantified from the results in FIG. 10.

FIG. 12 shows the protein M/antibody complex is stable after formation in vitro. In this experiment 1 μl of polyclonal serum (titer 1:2,564) containing an estimated 10 μg of immunoglobulin was used to incubate with protein M (2:1 molar ratio, 6.6 μg) for various durations before addition of AAV8-Luciferase. The negative control group contained neutralizing serum plus media, while the positive control groups contained either media plus PBS or protein M plus media. Incubation intervals of 72 h, 48 h, 24 h, 16 h, 4 h, 2 h, and 1 h were applied to all groups. All incubation durations showed protein M mediated protection of AAV8 from neutralization compared to the media plus PBS negative control. AAV8-Luciferase was added to the wells at the time of seeding (0 h) with 5×10⁴ HEK-293 cells and transduced at an MOI of 4,000 (n=3 technical replicates per well condition) in a 96-well plate format. Luciferase measurements were taken 48 h post transduction.

FIG. 13 shows the potency of neutralizing antibody escape by protein M in vivo is unstable. The same experiment from FIGS. 10 and 11 was performed but this time AAV8 was added at either 5 minutes post protein M administration, or 3 h post protein M administration in order to test the durability of NAb escape. AAV8-Luciferase signal was about ⅓ of the no-serum control group after waiting 3 h to administer AAV8 while the luciferase signal was roughly equivalent to the no-serum control group when waiting only 5 minutes to administer AAV8 after protein M. An estimated 2:1 ratio of protein M (6.3 mg) was delivered to mice after passive transfer but before AAV administration, and n=5 mice per group condition except for the 3 h interval group which contained n=3 mice. Luciferase signal from the liver was measured at 24 h post AAV administration.

FIG. 14 shows truncated protein M from M. genitalium (MG WT) is unstable at body temperature. Melting temperature (Tm) was determined with nano differential scanning fluoroscopy (NanoDSF). Inflection points of the first derivative indicate Tm. Proteins were recombinantly expressed in E. coli and purified with nickel column chromatography before measurement. A circular dichroism assay demonstrated that MG WT is stable for at least 2 h at 20° C., along with no visible precipitate forming. However, at 37° C. MG WT began to unfold after 15 minutes, along with production of visible precipitate. This indicates the protein is unstable near standard human body temperature. A temperature ramp from 0-100° C. demonstrated instantaneous unfolding of MG WT and a melting temperature (Tm) of about 41.2° C. Unfolding was accompanied by aggregation of a visible precipitate.

FIGS. 15A-15B show melting temperatures of truncated protein M from M. genitalium (MG WT) and M. pneumoniae (MP WT), as well as analogs of protein M that improved melting temperature over MG WT by at least 1 degree (A) or maintain the melting temperature of MG WT (B). Melting temperatures were determined by differential scanning fluoroscopy (NanoDSF) for WT and mutant protein analogs generated by small scale protein production from E. coli. The number of mutations and their corresponding amino-acid substitutions are listed to the right. The analogs demonstrate a range of thermostability, with multiple mutations resulting in an additive effect to increase melting temperature.

FIG. 16 shows example data using differential scanning fluoroscopy (DSF) to measure the melting temperature of MG WT and MG 29. Melting temperatures were determined for all analogs, with the results of MG WT (Tm=41.9° C.) and MG 29 (Tm=55.2° C.) pictured here. Inflection points of the first derivative indicate melting temperatures (Tm). Proteins were recombinantly expressed in E. coli and purified with nickel column chromatography before measurement.

FIGS. 17A-17C show soluble fraction assessment of protein M analogs determined by SDS-PAGE. Seven aliquots of each protein (0.4 mg/mL) were incubated at 37° C. for varying amounts of time. Precipitated protein was pelleted by centrifuging the samples at 15,000×G for 10 minutes before running the soluble fraction on an SDS-PAGE gel. Proteins were heated for either 0, 1, 4, 24, 48, 72, or 96 h prior to assessment. Results demonstrate MG WT (A) begins to precipitate out of solution between 1-4 h post heating. MG 27 (B) and MG 29 (A) remain soluble for over 72 h, while MG 31 (B) and MG 40 (C) remain soluble for over 24 h.

FIGS. 18A-18C show different protein M analogs block pooled human Intravenous Immunoglobulin Gamma (IVIG) to prevent AAV2-Luciferase vector neutralization during an in vitro neutralization assay: comparison of 4° C. vs. 37° C. thermal challenge for 1 h (A and B) or 24 h (C) incubation. Ratio of 4:1 protein M to IVIG. Relative light units generated by luciferase activity were measured from cell cultures 24 h after transduction by an AAV2-Luciferase reporter vector (2E8 vg) performed in triplicate in a 96-well plate format. Results were normalized to wells containing only AAV2 and phosphate buffered saline (PBS), white bar. AAV2 incubated with IVIG (12 μg) for 1 h demonstrated near complete neutralization of the AAV. Protein M analogs (16 μg) incubated at 4° C. prevented neutralization of AAV by IVIG when incubated together for 1 h prior to AAV. This was compared to protein M analogs incubated at 4° C. but without IVIG incubation, (not done for MG 8 and MG 24). MG WT and some analogs with lower melting temperatures did not protect AAV from neutralization when the analogs were thermally challenged at 37° C. prior to incubation with IVIG, while other analogs with higher Tm retained the ability to protect AAV from neutralization. Most mutant MG analogs retained neutralizing antibody blocking ability after 37° C. challenge for 1 h. However, MG 27, MG 29, and MG 46 prevented AAV neutralization after 37° C. challenge for 24 h.

FIG. 19 shows MG WT blocks NAbs for AAV re-dosing 1-month after primary AAV administration. Wild-type C57BL6 female mice were immunized via intraperitoneal administration of AAV8-GFP (1E9 vg), and serum was collected one month later to assess AAV neutralizing antibody titer in vitro. Mice were then administered AAV8-Luciferase reporter vectors intramuscularly (1E9 vg per leg), where AAV was formulated as a simple admixture with MG WT (33 μg per leg) to the mouse's right leg, or AAV was formulated with phosphate buffered saline as a vehicle control to the mouse's left leg. Luminescent imaging of the leg muscles was performed 2 weeks after AAV8-Luciferase administration. Three mice with an AAV neutralizing antibody titer of 1:10 demonstrated neutralization of the AAV luciferase reporter vector in the saline formulated leg, while the AAV luciferase reporter vector was protected from neutralizing antibodies in the MG WT formulated leg of the same mouse. Neutralization of AAV in the saline leg produced an average luciferase signal of 80% less than that of the MG WT formulated leg. This result demonstrates the ability to successfully re-dose AAV using protein M after generation of neutralizing antibodies elicited by a previous AAV dose.

FIG. 20 shows MG 29 blocks NAbs for AAV re-dosing 1-month after primary AAV administration. Wild-type C57BL6 female mice were immunized via intraperitoneal administration of AAV8-GFP (5E8 vg), and serum was collected one month later to assess AAV neutralizing antibody titer in vitro. Mice were then administered AAV8-Luciferase reporter vectors intramuscularly (2E9 vg per leg), where AAV was formulated as a simple admixture with the engineered analog MG 29 (500 μg per leg) to the mouse's right leg, or AAV was formulated with phosphate buffered saline as a vehicle control to the mouse's left leg. Luminescent imaging of the leg muscles was performed 2 weeks after AAV8-Luciferase administration. Three mice each with an AAV neutralizing antibody titer less than 1:100 (individually 1:8, 1:32, and 1:64) demonstrated neutralization of the AAV luciferase reporter vector in the saline formulated leg, while the AAV luciferase reporter vector was protected from neutralizing antibodies in the MG 29 formulated leg of the same mouse. Neutralization of AAV in the saline leg produced an average luciferase signal at least 98% less than that of the MG WT formulated leg. This result demonstrates the ability to successfully re-dose AAV using an engineered protein M analog after generation of neutralizing antibodies elicited by a previous AAV dose.

FIG. 21 shows engineered protein M analogs demonstrate different affinities for IgG. Affinity of specific protein M analogs were measured using biolayer inferometry (BLI). Binding constants (KD) are calculated using association and dissociation rates (kinetic analysis) over a protein M concentration range of 15.6 nM-250 nM. Results demonstrate enhanced or decreased affinity to IgG affixed to the BLI probe compared to MG WT.

FIG. 22 shows an example of BLI affinity data generated by kinetic analysis. The curve is used to predict the kinetic K_{D} value. The curve fit is overlaid on collected data.

FIG. 23 shows mutant stabilization success rate. The frequency that mutations predicted by Rosetta for MG WT increased (Tm+1° C.), decreased (Tm−1° C.), or had no effect on stability are shown. ‘Combined mutations’ represent constructs built by merging mutant constructs that stabilized MG WT individually.

FIG. 24 shows MP WT sequence conservation. The homology model of MP WT is depicted in two orientations, colored by conservation to the MG WT sequence according to the BLOSUM62 matrix. Residues are colored according to degree of conservation, ranging from white (identical) to black (significantly different). The homology model was built with PDB ID: 4NZR as a template.

FIG. 25 shows codon optimization of protein M DNA sequences enhances manufacturing yield. DNA plasmids encoding for MG WT protein were generated using either bacterial codons native to MG281 (Original PM), or codons optimized to both bacteria and human codon usage (Optimized PM). Three pooled bacterial colonies transformed by equivalent concentrations of either the Original PM plasmid or Optimized PM plasmid were cultured and propagated in equivalent growth media volumes (10 mL) for an equivalent overnight time period. Bacteria were pelleted and crude lysates were generated by freeze thaw and lysis in equivalent volumes of lysis buffer. Crude lysate was centrifuged and the supernatant collected for protein separation on an SDS-PAGE gel. Equal volumes from each lysate were run on the SDS-PAGE gel, transferred to a western blot, and then probed with a mouse antibody against the 6× Histidine tag present on the amino terminus of MG WT, followed by a goat anti-mouse secondary antibody conjugated to horseradish peroxidase (HRP). Band intensity of MG WT protein was measured using a luminol chemiluminescence assay. The resulting MG WT yield was calculated based on western blot band intensity normalized to the weight of the bacterial pellet. The Optimized PM plasmid generated roughly a 4 fold increase in MG WT protein yield when compared to the Original PM plasmid.

FIG. 26 shows improved pH stability of mutants. The melting temperature (Tm) was determined with NanoDSF at varying pH conditions. The protein was purified in PBS via SEC and buffer-exchanged into glycine (pH 2.5 & 3.5), acetate (pH 4.5 & 5.5), and phosphate (pH 6.5 and 7.5) buffers.

FIGS. 27A-27D show a sequence alignment of wild-type M. genitalium protein M amino acids 74-479 (SEQ ID NO:3) and modified protein M MG1 (SEQ ID NO:4), MG8 (SEQ ID NO:5), MG13 (SEQ ID NO:6), MG15 (SEQ ID NO:7), MG21 (SEQ ID NO:8), MG22 (SEQ ID NO:9), MG23 (SEQ ID NO:10), MG24 (SEQ ID NO:11), MG27 (SEQ ID NO:12), MG28 (SEQ ID NO:13), MG29 (SEQ ID NO:14), MG31 (SEQ ID NO:15), MG33 (SEQ ID NO:16), MG38 (SEQ ID NO:17), MG40 (SEQ ID NO:18), MG43 (SEQ ID NO:19), MG44 (SEQ ID NO:20), MG45 (SEQ ID NO:21), and MG46 (SEQ ID NO:22).

FIG. 28 shows a sequence alignment of wild-type M. genitalium protein M amino acids 74-479 (SEQ ID NO:3) and the equivalent fragment of M. pneumoniae protein M (SEQ ID NO:23).

FIG. 29 is a bar graph of luminescence after administration. FIG. 29 shows in vivo antibody neutralization 7 days after administration. Serum was collected and pooled from mice serially immunized with AAV8. In vitro neutralization assay determined that the anti-AAV8 neutralizing titer of the pooled serum to be roughly 1:10,000. Different quantities of the anti-AAV8 serum (determined by serum volume) were administered via IV injection to groups of AAV naive mice in a passive transfer experiment, followed 20-25 minutes later by IV injection of 2e10 viral genomes (vg) of AAV8-Luciferase reporter vector. Some groups of mice were given protein M via IV injection 5 minutes before the AAV administration. Non-invasive in vivo imaging of luciferase activity was performed 7 days later to quantify luminescence as a proxy of gene expression from the AAV8 vector, and any decrease in luminescence signal is indicative of AAV8-Luciferase neutralization by the serum. Mice given 0.0003 ul of serum failed to neutralize the AAV8 and resulted in nearly 100% luminescence signal as compared to naive mice without serum injected with 2e10 vg AAV8-Luciferase alone. Groups of mice given increasing quantities of neutralizing serum neutralized progressively more and more AAV8-Luciferase, where 0.001 ul to 0.003 ul serum neutralized approximately half of the AAV and 0.3 ul serum or greater neutralized over 99% of the AAV. Groups of mice administered 3 ul of serum, followed by MG88 or MG118 and then 2e10 vg AAV8-Luciferase, resulted in gene expression indicative of escape from neutralization due to suppression of neutralizing antibodies by the protein M fusion proteins MG29 (6.3 mg), MG29* (6.3 mg), and MGWT* (6.3 mg), MG118 (9 mg), and MG88 (6.3 mg).

FIG. 30 shows an in vitro neutralization assay where high titer anti-AAV8 serum from mice was titrated in 2-fold dilutions, and added to the well as indicated on the X-axis. Then the different Protein M analogs (MG29, MG66, MG71, and MG64) were added to the well in the molecular ratio indicated in the figure legend and incubated for 1 hour. Saline was added to the serum only well (black circles). Then AAV8-Luciferase (2×10¹⁰ viral genomes) was added to each well and incubated 1 hr, before adding 5×10⁴ Huh7 cells in serum free media. The cells were cultured 48 hrs and then the luciferase reporter signal, which is a proxy for neutralization, was read and quantified. These data demonstrate these analogs have neutralizing antibody blocking functionality, thereby shifting the neutralization curve, and that the MG64 Fc-Protein M fusion dimer (Fc-PM) shifts the curve greater than the Protein M monomer analogs.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 2nd Ed. (Cold Spring Harbor, N Y, 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, +0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1.

The term “adeno-associated virus” (AAV) in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J Virol. 78:6381-6388 and Table 1), which are also encompassed by the term “AAV.”

The parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC 001863, NC 001829, NC 001862, NC_000883, NC 001701, NC 001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC 001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73: 939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-:375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

A “chimeric” AAV nucleic acid capsid coding sequence or AAV capsid protein is one that combines portions of two or more capsid sequences. A “chimeric” AAV virion or particle comprises a chimeric AAV capsid protein.

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus and/or from a non-integrated episome, as well as any other form which the virus nucleic acid may take within the cell.

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g. U.S. Pat. No. 9,636,370 McCown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

As used herein, “transduction” of a cell by a virus vector (e.g., an AAV vector) means entry of the vector into the cell and transfer of genetic material into the cell by the incorporation of nucleic acid into the virus vector and subsequent transfer into the cell via the virus vector.

TABLE 1

| | GenBank |
|---|---|
| AAV | Accession |
| Serotypes/Isolates | Number |
| Clonal Isolates | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY388617 |
| AAV4 | NC_001829 |
| AAV5 | AY18065, |
| | AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

Unless indicated otherwise, “efficient transduction” or “efficient tropism,” or similar terms, can be determined by reference to a suitable positive or negative control (e.g., at least about 50%, 60%, 70%, 80%, 85%, 90%, 95% or more of the transduction or tropism, respectively, of a positive control or at least about 110%, 120%, 150%, 200%, 300%, 500%, 1000% or more of the transduction or tropism, respectively, of a negative control).

Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism for) tissues outside the CNS, e.g., liver, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., CNS cells).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides), but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical to the reference polypeptide or amino acid sequence. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention. In some embodiments, such fragments can comprise, consist essentially of, and/or consist of peptides having a length of less than about 4, 6, 8, 10, 12, 15, 20, 25, 30, 35, 40, 45, 50, 75, 100, 150, 200, 250, 300, 350, 400, 450, or 500 consecutive amino acids of a polypeptide or amino acid sequence according to the invention.

As used herein, a “functional fragment” is one that substantially retains at least one biological activity normally associated with that polypeptide (e.g., antibody binding). In “functional fragment” substantially retains all of the activities possessed by the unmodified polypeptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as antibody binding can be measured using assays that are well known in the art and as described herein.

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence (for example, in a wildtype sequence, including, e.g., a coding sequence for protein M) with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wild-type sequence due to one or more deletions, additions, substitutions, or any combination thereof.

As used herein, by “isolate” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus, is a sequence or nucleic acid, respectively, that is not naturally occurring in the virus. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “vector,” “virus vector,” “delivery vector” (and similar terms) in a specific embodiment generally refers to a virus particle that functions as a nucleic acid delivery vehicle, and which comprises the viral nucleic acid (i.e., the vector genome) packaged within the virion. Virus vectors according to the present invention comprise a chimeric AAV capsid according to the invention and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s), but need not be contiguous thereto. The TRs can be the same or different from each other. The vector genome can also contain a single ITR at its 3′ or 5′ end.

The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV ITR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV inverted terminal repeat” or “AAV ITR” may be from any AAV, including but not limited to serotypes 1, 2, 3, 4, 5, 6, 7, 8, 9, 10 or 11 or any other AAV now known or later discovered (see, e.g., Table 1). An AAV ITR need not have the native ITR sequence (e.g., a native AAV ITR sequence may be altered by insertion, deletion, truncation and/or missense mutations), as long as the ITR mediates the desired functions, e.g., replication, virus packaging, integration, and/or provirus rescue, and the like.

The terms “rAAV particle” and “rAAV virion” are used interchangeably here. A “rAAV particle” or “rAAV virion” comprises a rAAV vector genome packaged within an AAV capsid.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions.

As used herein, the term “amino acid” encompasses any naturally occurring amino acids, modified forms thereof, and synthetic amino acids, including non-naturally occurring amino acids.

Naturally occurring, levorotatory (L-) amino acids are shown in Table 2.

TABLE 2

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) or can be an amino acid that is modified by post-translation modification (e.g., acetylation, amidation, formylation, hydroxylation, methylation, phosphorylation or sulfatation).

TABLE 3
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., (2006) Annu. Rev. Biophys. Biomol. Struct. 35:225-49. These unnatural amino acids can advantageously be used to chemically link molecules of interest to the AAV capsid protein.

Conservative amino acid substitutions are known in the art. In particular embodiments, a conservative amino acid substitution includes substitutions within one or more of the following groups: glycine, alanine; valine, isoleucine, leucine; aspartic acid, glutamic acid; asparagine, glutamine; serine, threonine; lysine, arginine; and/or phenylalanine, tyrosine.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wildtype or synthetic. A wildtype large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

By “substantially retain” a property, it is meant that at least about 75%, 85%, 90%, 95%, 97%, 98%, 99% or 100% of the property (e.g., activity or other measurable characteristic) is retained.

### Methods of Using Protein M and Derivatives Thereof to Bind Antibodies

One aspect of the present invention relates to a method of inhibiting neutralization of a heterologous agent by neutralizing antibodies upon administration of the heterologous agent to a subject, comprising administering to the subject an effective amount of Mycoplasma protein M or a functional fragment or derivative thereof or a fusion protein comprising an effective amount of Mycoplasma protein M or a functional fragment or derivative thereof, thereby inhibiting neutralization of the heterologous agent.

Another aspect of the invention relates to a method of expressing a polypeptide or functional nucleic acid in a subject, comprising administering to the subject (a) a nucleic acid delivery vector encoding the polypeptide or functional nucleic acid, and (b) an effective amount of Mycoplasma protein M or a functional fragment or derivative thereof or a fusion protein comprising Mycoplasma protein M or a functional fragment or derivative thereof, thereby expressing the polypeptide or functional nucleic acid in the subject.

A further aspect of the invention relates to a method of editing a gene in a subject, comprising administering to the subject (a) a gene editing complex, and (b) an effective amount of Mycoplasma protein M or a functional fragment or derivative thereof or a fusion protein comprising an effective amount of Mycoplasma protein M or a functional fragment or derivative thereof, thereby expressing the polypeptide or functional nucleic acid in the subject.

As used herein, the term “heterologous agent” refers to an agent that is not naturally found in the subject to which the agent is to be administered. The term also includes recombinant or synthetic versions of agents that are naturally found in the subject. The heterologous agent may be one for which neutralizing antibodies are present in the subject prior to administration of the heterologous agent or one that is likely to raise neutralizing antibodies upon administration to the subject. The heterologous agent may be one that has never been administered to the subject. The heterologous agent may be one that has previously been administered to the subject.

As used herein, the term “neutralizing antibodies” refers to antibodies that specifically bind to a heterologous agent and inhibit one or more biological activities of the heterologous agent after it has been administered to a subject.

In some embodiments, the heterologous agent may be a nucleic acid delivery vector, e.g., a viral vector or a non-viral vector. In some embodiments, the viral vector is an oncolytic vector. In some embodiments, the viral vector is an adeno-associated virus, retrovirus, lentivirus, poxvirus, alphavirus, baculovirus, vaccinia virus, herpes virus, Epstein-Barr virus, polio virus, or adenovirus vector. In some embodiments, the non-viral vector is a plasmid, liposome, electrically charged lipid, nucleic acid-protein complex, or biopolymer.

In some embodiments, the heterologous agent is a gene editing complex, e.g., a CRISPR complex.

In some embodiments, the heterologous agent is a protein or nucleic acid. In some embodiments, the protein is an enzyme, a regulatory protein, or a structural protein, e.g., one that can substitute for a missing or defective protein in a subject. In some embodiments, the nucleic acid is a functional nucleic acid, e.g., an antisense nucleic acid or an inhibitory RNA.

The effective amount of protein M or a functional fragment or derivative thereof or a fusion protein comprising an effective amount of Mycoplasma protein M or a functional fragment or derivative thereof is an amount that at least partially blocks the inhibition of the heterologous agent by neutralizing antibodies and/or outcompetes self-neutralizing antibodies ability to bind to the heterologous agent. In some embodiments, the effective amount of protein M is an amount sufficient to inhibit neutralization by at least about 50%, 60%, 70%, 80%, 90%, 91%, 92%, 93%, 94%, 95%, 96%, 97%, 98, 99%, 99.5%, or 99.9%. In some embodiments, the effective amount of protein M or a functional fragment or derivative thereof or a fusion protein comprising an effective amount of Mycoplasma protein M or a functional fragment or derivative thereof is an amount sufficient to produce a ratio of protein M to total immunoglobulin in the subject of about 0.5:1 to about 8:1 on a molar basis or any range therein, e.g., about 0.5:1, 1:1, 1.5:1, 2:1, 2.5:1, 3:1, 3.5:1, 4:1, 4.5:1, 5.5:1, 6:1, 6.5:1, 7:1, 7.5:1, or 8:1 or any range therein. In other embodiments, the ratio is from about 8:1 to about 50:1. In some embodiments, the ratio is about 0.5:1 to about 6:1, about 0.5:1 to about 4:1, about 0.5:1 to about 2.5:1, about 0.5:1 to about 2:1, about 1:1 to about 8:1, about 1.5:1 to about 8:1, or about 2:1 to about 8:1. In one embodiment, the ratio is about 1:1 to about 3:1, e.g., about 2:1.

In some embodiments, the administration comprises from about 1e9 vg/kg to about 1e14 vg/kg, from about 1e10 vg/kg to about 1e13 vg/kg, or from about Tell vg/kg to about 1e12 vg/kg of AAV, from about 1 μl to about 6 L of serum, and from about 1 mg/kg to about 1000 mg/kg, from about 10 mg/kg to about 900 mg/kg, from about 25 mg/kg to about 800 mg/kg, from about 50 mg/kg to about 600 mg/kg, from about 100 mg/kg to about 500 mg/kg, or from about 200 mg/kg to about 400 mg/kg of Protein M.

Total immunoglobulin may be total serum immunoglobulin (e.g., for systemic administration of protein M). Total immunoglobulin may be the total level in a localized fluid or tissue (e.g., for specific delivery to the eye, ear, lung, brain, muscle, joint, etc.). Total immunoglobulin may be measured by any technique known in the art, such as by performing an ELISA on serum using either an antibody that binds the Fc region of immunoglobulins or by using Protein A or G to bind immunoglobulins. Additionally, for mice it is known that their serum contains between 5 mg/ml to 10 mg/ml immunoglobulin. The normal range for serum immunoglobulin in humans is 8-10 mg/ml. For in vivo estimates, the high end of 10 mg/ml can be used to calculate the ratio. Local immunoglobulin content can be estimated based on tissue weight (40 mL of serum per 1 kg weight), or concentration of Ig in specific body fluids and the body fluid volume in that organ if it is less than blood serum (e.g., eye, cerebrospinal fluid).

The protein M may be administered to the subject by any schedule found to be effective to block inhibition of the heterologous agent by neutralizing antibodies. In some embodiments, the protein M or a functional fragment or derivative thereof or a fusion protein comprising an effective amount of Mycoplasma protein M or a functional fragment or derivative thereof is administered to the subject prior to administration of the heterologous agent, e.g., at least about 1, 5, 10, 15, 20, 30, 40, or 50 minutes or at least about 1, 2, 3, 4, 5, 6, 12, 18, 24. 48, or 72 hours prior to administration of the heterologous agent. In some embodiments, the protein M or a functional fragment or derivative thereof or a fusion protein comprising an effective amount of Mycoplasma protein M or a functional fragment or derivative thereof is administered to the subject concurrently with administration of the heterologous agent. As used herein, the term “concurrently” means sufficiently close in time to produce a combined effect (that is, concurrently can be simultaneously, or it can be two or more events occurring within a short time period before or after each other).

In some embodiments, the heterologous agent is combined with the protein M or a functional fragment or derivative thereof or a fusion protein comprising an effective amount of Mycoplasma protein M or a functional fragment or derivative thereof prior to administration to the subject, e.g., the two components are mixed together prior to administration in a single composition. The heterologous agent may be combined with the protein M or a functional fragment or derivative thereof or a fusion protein comprising an effective amount of Mycoplasma protein M or a functional fragment or derivative thereof at least about 1, 5, 10, 15, 20, 30, 40, or 50 minutes or at least about 1, 2, 3, 4, 5, 6, 12, 18, or 24 hours prior to administration to the subject. In other embodiments, the protein M or a functional fragment or derivative thereof or a fusion protein comprising an effective amount of Mycoplasma protein M or a functional fragment or derivative thereof and the heterologous agent are administered in separate compositions.

In some embodiments, it may be necessary to administer the heterologous agent and/or the protein M or a functional fragment or derivative thereof or a fusion protein comprising an effective amount of Mycoplasma protein M or a functional fragment or derivative thereof to the subject more than once to provide a therapeutic or otherwise beneficial effect. The protein M or a functional fragment or derivative thereof or a fusion protein comprising an effective amount of Mycoplasma protein M or a functional fragment or derivative thereof may be administered, e.g., 1, 2, 3, 4 or more times. The administrations may be days, weeks, months, or years apart (e.g., from about two days to about 10 years or more). In some embodiments, the administration decreases the amount of antibody cycling and the onset of the decrease is from about 1 hour to about 3 hours. In some embodiments, the administration decreases the amount of antibody cycling and the onset of the decrease is from about 5 minutes to about 8 weeks, from about 5 minutes to about 1 hour, from about 1 hour to about 2 days, or from about 2 days to about 8 weeks. In some embodiments, the protein M or a functional fragment or derivative thereof or a fusion protein comprising an effective amount of Mycoplasma protein M or a functional fragment or derivative thereof is administered to the subject each time the heterologous agent is administered to the subject, e.g., in the same manner as described above, e.g., before or currently with the heterologous agent. The use of protein M with each administration of the heterologous agent may inhabit the effect of NAb against the heterologous agent which is often an issue upon readministration. In some embodiments, the same protein M or a functional fragment or derivative thereof or a fusion protein comprising an effective amount of Mycoplasma protein M or a functional fragment or derivative thereof is administered each time. In other embodiments, a different protein M or a functional fragment or derivative thereof or a fusion protein comprising an effective amount of Mycoplasma protein M or a functional fragment or derivative thereof is administered each time, e.g., a different modified protein M as describe further below. Without being bound by theory, it is thought that the use of a different protein M derivative with each administration may limit the effect of inhibitory antibodies against protein M that may occur with readministration of the same protein. It is also thought that administration of saturating doses of protein M or a functional fragment or derivative thereof or a fusion protein comprising an effective amount of Mycoplasma protein M or a functional fragment or derivative thereof will outcompete any inhibitory antibodies to protein M and prevent antigen recognition. In some embodiments, administrations outcompete self-neutralizing antibodies.

The ability of protein M or a functional fragment or derivative thereof or a fusion protein comprising an effective amount of Mycoplasma protein M or a functional fragment or derivative thereof to non-specifically bind antibodies advantageously may be used in other methods where it is beneficial to inhibit antibody binding to antigen, e.g., where immunosuppression is desirable or where excess antibodies are present.

An additional aspect of the invention relates to a method of treating an autoimmune disease in a subject in need thereof, comprising administering to the subject a therapeutically effective amount of Mycoplasma protein M or a functional fragment or derivative thereof or a fusion protein comprising an effective amount of Mycoplasma protein M or a functional fragment or derivative thereof, thereby treating the autoimmune disease.

The term “autoimmune disease,” as used herein, refers to any disorder associated with an autoimmune reaction. Examples include, without limitation, multiple sclerosis, Crohn's disease, ulcerative colitis, systemic lupus erythematosus, rheumatoid arthritis, inflammatory bowel syndrome, irritable bowel syndrome, uveitis, insulin-dependent diabetes mellitus, hemolytic anemias (e.g., warm autoimmune hemolytic anemia), rheumatic fever, Goodpasture's syndrome, Guillain-Barre syndrome, psoriasis, thyroiditis, Graves' disease, myasthenia gravis, glomerulonephritis, autoimmune hepatitis, immune thrombocytopenic purpura, acute inflammatory demyelinating polyneuropathy, antibody-mediated rapidly progressive glomerulonephritis, chronic inflammatory demyelinating polyradiculoneuropathy, hyper-viscosity syndrome, recurrent focal segmental glomerulosclerosis, neuromyelitis optica spectrum disorder, cryoglobulinemia, autoimmune encephalitis, autoimmune neuropathy, ANCA vasculitis, autoimmune neutropenia, anti-GBM disease, pemphigus vulgaris, Sjogren's syndrome, lupus nephritis, and immune cytopenias.

Another aspect of the invention relates to a method of treating a disorder associated with excess antibodies in a subject in need thereof, comprising administering to the subject a therapeutically effective amount of Mycoplasma protein M or a functional fragment or derivative thereof or a fusion protein comprising an effective amount of Mycoplasma protein M or a functional fragment or derivative thereof, thereby treating the disorder associated with excess antibodies. The term “disorder associated with excess antibodies,” as used herein, refers to any disorder wherein the cause or at least one symptom of the disorder is due to greater than average levels of antibodies in the blood or elsewhere in the body. Examples include, without limitation, multiple myeloma, monoclonal gammopathy of undetermined significance (MGUS), Waldenstrom macroglobulinemia, cytokine release syndrome, or acute autoimmune attacks such as severe autoimmune vasculitis with sudden onset. The method may also be useful for an acute blockade of all antibodies to rapidly stop an autoimmune event such as cytokine release syndrome or acute autoimmune attacks such as severe autoimmune vasculitis with sudden onset, or preventing damage to transplant tissue and/or transplant organ caused by antibody mediated immune complex formation.

For any of the methods of the invention, the protein M or a functional fragment or derivative thereof or a fusion protein comprising an effective amount of Mycoplasma protein M or a functional fragment or derivative thereof may be administered to the subject by any route of administration found to be effective (e.g., local or systemic).

The most suitable route will depend on the subject being treated and the disorder or condition being treated. In some embodiments, the protein M or a functional fragment or derivative thereof or a fusion protein comprising an effective amount of Mycoplasma protein M or a functional fragment or derivative thereof is administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

The protein M or a functional fragment or derivative thereof or a fusion protein comprising an effective amount of Mycoplasma protein M or a functional fragment or derivative thereof may be delivered or targeted to any tissue or organ in the subject. In some embodiments, the protein M or a functional fragment or derivative thereof or a fusion protein comprising an effective amount of Mycoplasma protein M or a functional fragment or derivative thereof is administered to, e.g., a skeletal muscle, a smooth muscle, the heart, the diaphragm, the airway epithelium, the liver, the kidney, the spleen, the pancreas, the skin, the lung, the ear, and the eye. In some embodiments, the protein M or a functional fragment or derivative thereof or a fusion protein comprising an effective amount of Mycoplasma protein M or a functional fragment or derivative thereof is administered to a diseased tissue or organ, e.g., a tumor.

In some embodiments, the heterologous agent and the protein M or a functional fragment or derivative thereof or a fusion protein comprising an effective amount of Mycoplasma protein M or a functional fragment or derivative thereof are administered by the same route. In other embodiments, the heterologous agent and the protein M or a functional fragment or derivative thereof or a fusion protein comprising an effective amount of Mycoplasma protein M or a functional fragment or derivative thereof are administered by different routes, e.g., the protein M or a functional fragment or derivative thereof or a fusion protein comprising an effective amount of Mycoplasma protein M or a functional fragment or derivative thereof is administered intravenously and the heterologous agent is administered locally to a target tissue or organ (e.g., diseased tissue or organ).

Any of the above methods may further comprise administering to the subject an additional treatment for reducing antibody concentration or inhibiting antibody function in the subject. The additional treatment may be any method known in the art and incudes, without limitation, plasmapheresis, administration of an antibody digesting enzyme such as IdeS or IdeZ, administration of an antibody that binds the FcRn receptor, administration of an Fc fragment that binds the FcRn receptor, administration of an immunosuppressant drug (e.g., corticosteroids (e.g., prednisone, budesonide, prednisolone), Janus kinase inhibitors (e.g., tofacitinib), calcineurin inhibitors (e.g., cyclosporine, tacrolimus), mTOR inhibitors (e.g., sirolimus, everolimus), IMDH inhibitors (e.g., azathioprine, leflunomide, mycophenolate), or biologics (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab, daclizumab), administration of an antibody that binds CD19 or an antibody that binds CD20 on B cells and depletes them or other treatments designed to inhibit or destroy B cells (e.g., splenectomy, chemotherapy, immunotherapy, radiation therapy). The additional treatment may be administered before, during, and/or after administration of the protein M or a functional fragment or derivative thereof or a fusion protein comprising an effective amount of Mycoplasma protein M or a functional fragment or derivative thereof.

The ability of protein M or a functional fragment or derivative thereof or a fusion protein comprising an effective amount of Mycoplasma protein M or a functional fragment or derivative thereof to non-specifically bind antibodies advantageously may be used in purification methods. While purification of antibodies often relies on agents that bind to the Fc region of the antibody (such as protein A and protein G), protein M non-specifically binds to the variable region of the antibody. Thus, protein M can be used to isolate antibody fragments and antibody derivatives that do not contain an Fc region (such as single chain variable fragments) and other molecules that incorporate an antibody variable region.

Thus, one aspect of the invention relates to a method of isolating a compound comprising an antibody light chain variable region and/or heavy chain variable region from a sample, the method comprising contacting the compound with the modified Mycoplasma protein M or a functional fragment thereof or a fusion protein comprising an effective amount of Mycoplasma protein M or a functional fragment or derivative thereof of the invention attached to a solid support, then eluting the compound from the modified Mycoplasma protein M or a functional fragment thereof or a fusion protein comprising an effective amount of Mycoplasma protein M or a functional fragment or derivative thereof. In some embodiments, the compound comprising an antibody light chain variable region and/or heavy chain variable region is an antibody or an antigen-binding fragment thereof. In some embodiments, the compound comprising an antibody light chain variable region and/or heavy chain variable region is an antibody derivative, an immunoglobulin scaffold, or the like. The modified protein M or a functional fragment thereof or a fusion protein comprising an effective amount of Mycoplasma protein M or a functional fragment or derivative thereof of the invention is advantageous over wild-type protein M due to increased thermostability. This allows the protein M to be reusable for multiple purifications and permits the use of elution conditions that would destabilize wild-type protein M.

The method may be carried using techniques well known in the art of affinity purification. The solid support may be any material that is suitable for affinity chromatography or batch purification. Suitable materials include, without limitation, agarose, polyacrylamide, dextran, cellulose, polysaccharide, nitrocellulose, silica, alumina, aluminum oxide, titania, titanium oxide, zirconia, styrene, polyvinyldifluoride nylon, copolymer of styrene and divinylbenzene, polymethacrylate ester, derivatized azlactone polymer or copolymer, glass, or cellulose. In some embodiments, the solid support is a resin. In some embodiments, the solid support is a bead or particle. In some embodiments, the solid support is a surface, e.g., of a plate, vial, or column.

The contacting step may be carried out by any suitable method, e.g., by passing a sample comprising the compound over the modified Mycoplasma protein M or a functional fragment thereof or a fusion protein comprising an effective amount of Mycoplasma protein M or a functional fragment or derivative thereof in a column or incubating the composition comprising the compound with the modified Mycoplasma protein M or a functional fragment thereof or a fusion protein comprising an effective amount of Mycoplasma protein M or a functional fragment or derivative thereof in a vessel or the well of a plate. The contacting step may be carried out for a sufficient amount of time to allow the compound to bind to the modified Mycoplasma protein M or a functional fragment thereof or a fusion protein comprising an effective amount of Mycoplasma protein M or a functional fragment or derivative thereof. After washing, centrifugation, or other forms of separation of the compound bound to the modified Mycoplasma protein M or a functional fragment thereof or a fusion protein comprising an effective amount of Mycoplasma protein M or a functional fragment or derivative thereof from other components in the sample, the compound is eluted from the modified Mycoplasma protein M or a functional fragment thereof or a fusion protein comprising an effective amount of Mycoplasma protein M or a functional fragment or derivative thereof. The elution may be carried out by any method known in the art, e.g., a change in ion concentration, temperature, etc. In one embodiment, the elution is carried out by a change in pH. The modified Mycoplasma protein M or a functional fragment thereof of the present invention advantageously is stable over a wider range of pH than wild-type protein M. This allows the modified protein M to remain stable at lower pHs that permit elution of the compound.

In some embodiments, the contacting step is carried out in a binding buffer (e.g., at neutral pH) and the elution is carried out with a low pH buffer (e.g., 0.1 M glycine pH 2-3.5 or 0.1 M acetate pH 3.5-4.5) into a neutralization buffer (e.g., a high-ionic strength alkaline buffer such as 1 M phosphate or 1 M Tris at pH 8-9).

A further aspect of the invention relates to the modified Mycoplasma protein M or a functional fragment thereof or a fusion protein comprising an effective amount of Mycoplasma protein M or a functional fragment or derivative thereof attached to a solid support as described above. The modified Mycoplasma protein M or a functional fragment thereof or a fusion protein comprising an effective amount of Mycoplasma protein M or a functional fragment or derivative thereof may be attached to the solid support by any means known in the art, e.g., covalently linked, e.g., using a linker molecule.

The ability of protein M or a functional fragment or derivative thereof or a fusion protein comprising an effective amount of Mycoplasma protein M or a functional fragment or derivative thereof to non-specifically bind antibodies advantageously may be used in any immunoassay that involves a step of binding an antibody or fragment or derivative thereof or a fusion protein comprising an effective amount of Mycoplasma protein M or a functional fragment or derivative thereof.

Thus, one aspect of the invention relates to a method of performing an immunoassay, the method comprising using the modified Mycoplasma protein M or a functional fragment thereof or a fusion protein comprising an effective amount of Mycoplasma protein M or a functional fragment or derivative thereof of the invention to bind a compound comprising an antibody light chain variable region and/or heavy chain variable region.

The modified Mycoplasma protein M or a functional fragment thereof or a fusion protein comprising an effective amount of Mycoplasma protein M or a functional fragment or derivative thereof can substitute for any generic or specific antibody binding molecule, e.g., protein A, protein G, or a secondary antibody. The modified Mycoplasma protein M or a functional fragment thereof or a fusion protein comprising an effective amount of Mycoplasma protein M or a functional fragment or derivative thereof may be labeled as is well known in the art, e.g., for radioactive, chemiluminescent, or enzymatic detection.

Examples of immunoassays include, without limitation, radio-immunoassays (RIA), enzyme-linked immunosorbent assays (ELISA) assays, enzyme immunoassays (EIA), sandwich assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, immunoprecipitation, competitive binding assays, and immunohistochemical staining.

### Protein M and Derivatives Thereof

The protein M or a functional fragment or derivative thereof used in the methods of the invention may be from any mycobacterial species that produces a protein M that binds to antibodies. In some embodiments, the protein M or a functional fragment or derivative thereof is from Mycoplasma genitalium, Mycoplasma pneumoniae, or Mycoplasma penetrans.

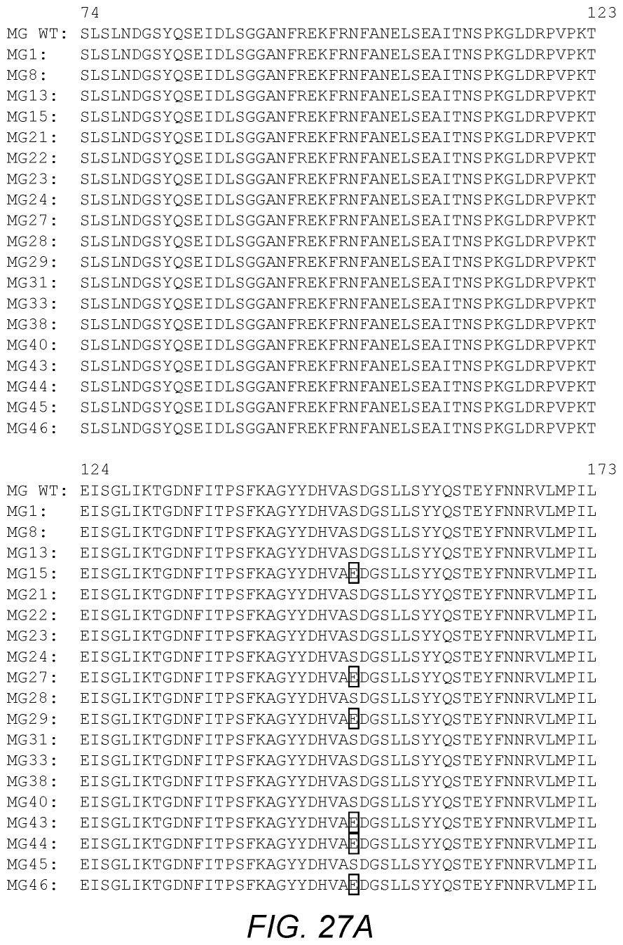

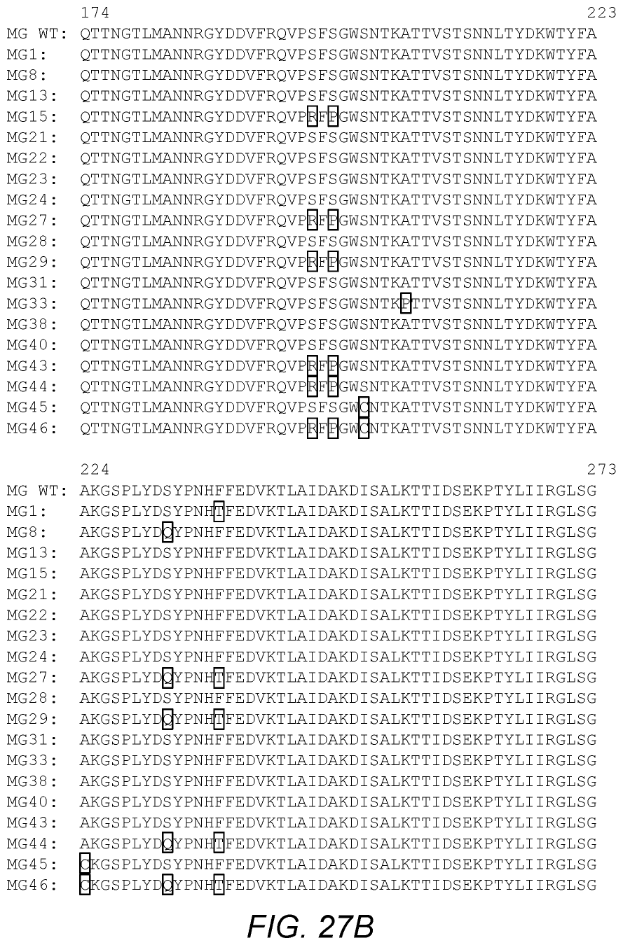

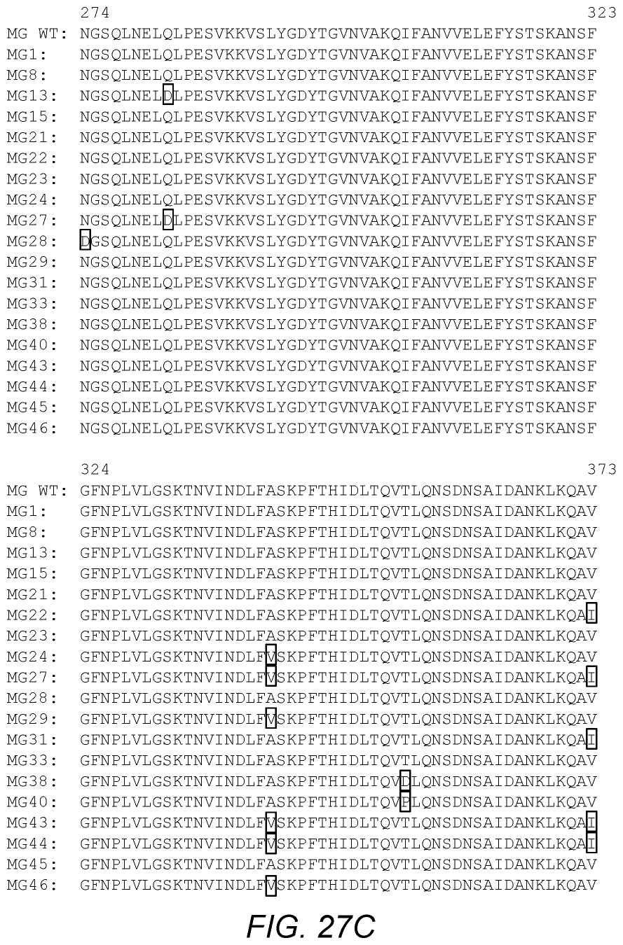

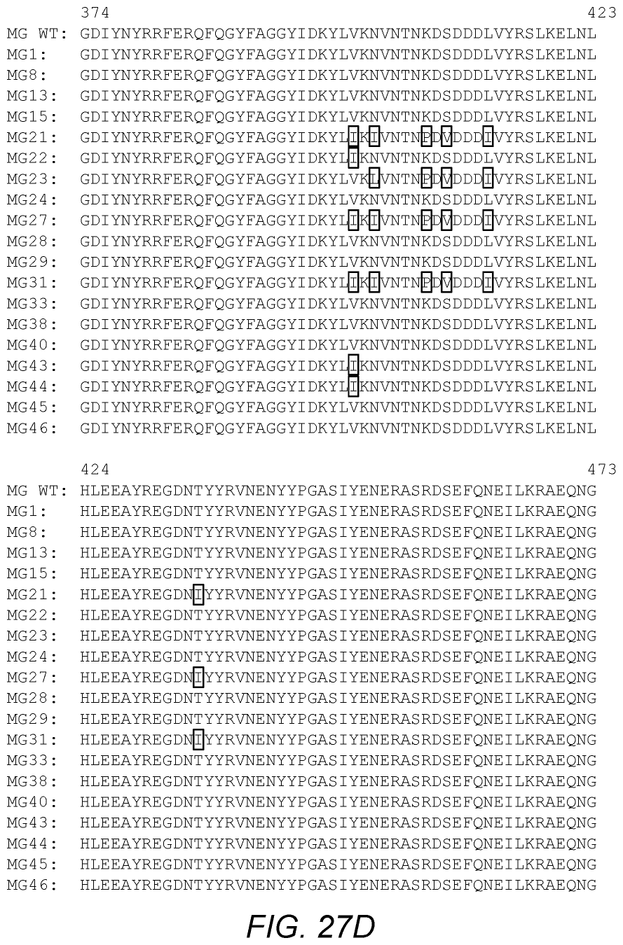

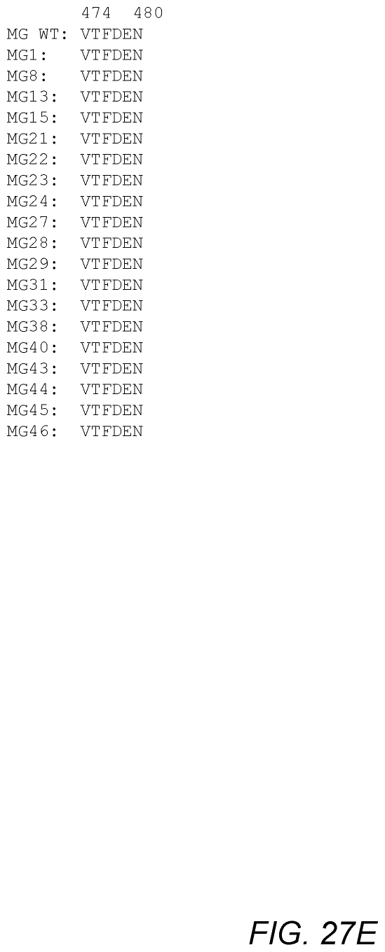

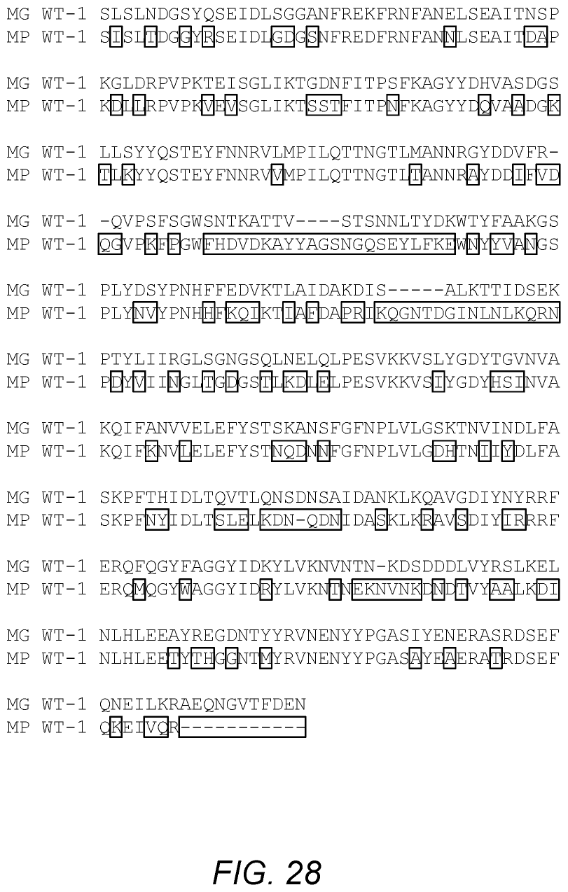

In some embodiments, the protein M or a functional fragment or derivative thereof may be any of the protein M sequences described in PCT Publication No. WO 2014/014897 and US Publication No. 2017/0320921, incorporated by reference herein in their entirety. In some embodiments, the protein M or a functional fragment or derivative thereof is a M. genitalium protein M (MG281, SEQ ID NO:1) or a functional fragment or derivative thereof (e.g., the fragment shown in SEQ ID NO:3 or a derivative thereof). In some embodiments, the protein M or a functional fragment or derivative thereof is a M. pneumoniae protein M (MPN400, SEQ ID NO:23) or a functional fragment or derivative thereof (e.g., the fragment shown in SEQ ID NO:24 or a derivative thereof). In some embodiments, the protein M or a functional fragment or derivative thereof is a Mycoplasma penetrans protein M or a functional fragment or derivative thereof. In some embodiments, the protein M or a functional fragment or derivative thereof is a protein M fragment or a derivative of the fragment, e.g., a fragment that does not contain the transmembrane domain and/or does not contain the C-terminus, e.g., a functional fragment comprising, consisting essentially of, or consisting of about amino acid residues 17-537, 37-556, 37-482, 37-468, 37-442, 74-468, 74-479, 74-482, 74-468, 74-442, or 74-556 of M. genitalium protein M (SEQ ID NO:1) or the equivalent residues from another protein M. The term “about” as applied to the termini of each of the listed fragments means that one or both of the terminal residues may be varied to a small extent, e.g., by about 5, 4, 3, or 2 amino acids on either side of the recited residue. The equivalent residues from another protein M can readily be determined by the skilled artisan by performing a sequence alignment between M. genitalium protein M and the other protein M. For example, FIG. 27 showing a sequence alignment of wild-type M. genitalium protein M amino acids 74-479 (SEQ ID NO:3) and the equivalent fragment of M. pneumoniae protein M (SEQ ID NO:24). In some embodiments, the protein M or a functional fragment or derivative thereof comprises, consists essentially of, or consists of the amino acid sequence of SEQ ID NO:2, which is a soluble form of protein M (amino acid residues 37-556 of SEQ ID NO:1) with an N-terminal 6-His tag followed by a thrombin cleavage site.

As used herein, the term “derivative” is used to refer to a polypeptide which differs from a naturally occurring Protein M or Protein M functional fragment by minor modifications to the naturally occurring polypeptide, but which significantly retains a biological activity of Protein M. Minor modifications include, without limitation, changes in one or a few amino acid side chains, changes to one or a few amino acids (including deletions, insertions, and/or substitutions), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol. The term “substantially retains,” as used herein, refers to a fragment, derivative, or other variant of a polypeptide that retains at least about 20% of the activity of the naturally occurring polypeptide (e.g., antibody binding), e.g., about 30%, 40%, 50% or more. In some embodiments, the derivative of Protein M or Protein M functional fragment contains mutations (deletions, insertions, and/or substitutions in any combination) of 20 or fewer amino acid residues, e.g., 20, 19, 18, 17, 16, 15, 14, 13, 12, 11, 10, 9, 8, 7, 6, 5, 4, 3, or 2 or fewer mutations. In some embodiments, the protein M derivative comprises an amino acid sequence that is at least about 80%, 85%, 90%, 91%, 92%, 93%, 94%, 95%, 96%, 97%, 98%, 99%, 99.5%, or 99.9% identical to the amino acid sequence of SEQ ID NO:1, SEQ ID NO:2, or SEQ ID NO:3 of M. pneumoniae protein M or the wild-type sequence of another Mycoplasma Protein M or a functional fragment thereof.

In some embodiments, the protein M or a functional fragment or derivative thereof can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases. Such blocking agents can include, without limitation, additional related or unrelated peptide sequences that can be attached to the amino and/or carboxyl terminal residues of the protein to be administered. This can be done either chemically during the synthesis of the protein or by recombinant DNA technology by methods familiar to artisans of average skill. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the proteins can be covalently or noncovalently coupled to pharmaceutically acceptable “carrier” proteins prior to administration.

In one aspect of the invention, the protein M derivative is a modified Mycoplasma protein M or a functional fragment thereof comprising mutations that increase or at least maintain the thermostability of protein M. These modified protein M derivatives have increased suitability for use in in vivo methods and other methods that require elevated temperatures (e.g., about 37° C.) where wild-type protein M may denature.

In some embodiments, the protein M derivative is a modified Mycoplasma protein M or a functional fragment thereof, having one or more amino acid mutations that increase or maintain thermostability of the Mycoplasma protein M or a functional fragment thereof relative to wild-type Mycoplasma protein M or a functional fragment thereof. In some embodiments, the modified protein M or a functional fragment thereof has an increased melting temperature (Tm) that is at least 0.5° C. than the Tm of wild-type protein M or a functional fragment thereof, e.g., 0.5° C., 1.0° C., 1.5° C., 2.0° C., 2.5° C., 3.0° C., 3.5° C., 4.0° C., 4.5° C., 5.0° C., 5.5° C., 6.0° C., 6.5° C., 7.0° C., 7.5° C., 8.0° C., 8.5° C., 9.0° C., 9.5° C., 10.0° C., 11.0° C., 12.0° C., 13.0° C., 14.0° C., 15.0° C., 16.0° C., 17.0° C., 18.0° C., 19.0° C., 20.0° C., 25.0° C., 30.0° C. or more higher. In some embodiments, the modified protein M or a functional fragment thereof has a Tm that is maintained (i.e., is within 0.5° C.) relative to the Tm of wild-type protein M or a functional fragment thereof. The Tm may be measured by differential scanning fluoroscopy or any other suitable technique. The Tm of wild type Mycoplasma genitalium protein M is about 41.9° C. and the Tm of wild type Mycoplasma pneumoniae protein M is about 44.1° C.

In some embodiments, the modified Mycoplasma protein M or a functional fragment thereof may have 1, 2, 3, 4, 5, 6, 7, 8, 9, 10, 11, 12, 13, 14, 15, 16, 17, 18, 19, or 20 or more mutations. In some embodiments, the modified Mycoplasma protein M or a functional fragment thereof may have 20, 19, 18, 17, 16, 15, 14, 13, 12, 11, 10, 9, 8, 7, 6, 5, 4, 3, 2, or fewer mutations.

In some embodiments, the modified Mycoplasma protein M or a functional fragment thereof is derived from protein M of Mycoplasma genitalium, Mycoplasma pneumoniae, or Mycoplasma penetrans.

In some embodiments, the modified Mycoplasma protein M or a functional fragment thereof is a fragment from about residue 74 (e.g., residue 69, 70, 71, 72, 73, 74, 75, 76, 77, 78, 79) to about residue 479 (e.g., residue 474, 475, 476, 477, 478, 479, 480, 481, 482, 483, 484) of M. genitalium protein M (SEQ ID NO:3) or the equivalent residues of M. pneumoniae protein M (SEQ ID NO:24).

In some embodiments, the one or more mutations are located in portions of protein M that are known to effect thermostability. In some embodiments, the one or more mutations are not located in proteins of protein M that are known to have other roles in the biological activity of protein M. In one embodiment, the one or more mutations are not at a residue within 5 Å of the antibody-binding site of protein M (i.e., residues 95, 99, 102, 103, 105, 106, 107, 109, 110, 114, 116, 117, 118, 119, 120, 144, 158, 160, 161, 162, 163, 177, 178, 179, 180, 181, 186, 187, 188, 191, 321, 338, 340, 341, 345, 381, 384, 387, 388, 389, 390, 391, 392, 393, 394, 395, 396, 397, 426, 427, 429, 436, 438, 439, 440, 441, 442, 444, 445, 446, 447, 448, 449, 452, 453, 455, 456, 457, 462, 466) of M. genitalium protein M (SEQ ID NO: 1) or the equivalent residues of M. pneumoniae protein M (SEQ ID NO:23). In one embodiment, the one or more mutations are not at a residue within 5 Å of the antibody-binding site of protein M (i.e., residues 100, 104, 107, 108, 110, 111, 112, 114, 115, 119, 121, 122, 123, 124, 125, 149, 163, 165, 166, 167, 168, 182, 183, 184, 185, 186, 192, 193, 196, 337, 338, 354, 356, 357, 399, 402, 404, 405, 406, 407, 408, 409, 410, 411, 412, 442, 443, 445, 454, 455, 456, 457, 458, 460, 461, 462, 463, 464, 465, 468, 469, 472, 473, 478) of M. pneumoniae protein M (SEQ ID NO:23). In one embodiment, the one or more mutations is not at any of residues 469-479 of M. genitalium protein M (SEQ ID NO:1) or the equivalent residues of M. pneumoniae protein M (SEQ ID NO:23).

The present inventors have used computational analysis to identify residues in protein M that are predicted to increase or maintain the Tm of the protein when mutated. Thus, in some embodiments, the one or mutations are at residue 78, 81, 83, 84, 85, 89, 90, 91, 92, 93, 94, 96, 97, 100, 101, 108, 111, 112, 113, 122, 123, 125, 126, 127, 128, 130, 131, 133, 134, 136, 137, 139, 141, 142, 146, 147, 148, 149, 150, 153, 154, 155, 156, 164, 167, 170, 175, 176, 184, 185, 189, 192, 193, 196, 198, 201, 202, 204, 205, 206, 207, 209, 211, 215, 218, 220, 224, 225, 226, 227, 231, 232, 234, 235, 236, 237, 239, 241, 243, 244, 245, 246, 247, 249, 250, 252, 253, 254, 255, 256, 257, 258, 259, 264, 269, 270, 272, 274, 275, 276, 279, 282, 284, 286, 287, 288, 291, 297, 299, 300, 302, 303, 304, 305, 307, 308, 309, 310, 311, 313, 317, 318, 319, 320, 322, 326, 327, 329, 331, 332, 333, 335, 337, 342, 343, 347, 348, 351, 354, 355, 357, 358, 359, 360, 361, 362, 363, 367, 369, 370, 371, 372, 373, 374, 375, 378, 385, 399, 400, 401, 402, 405, 406, 407, 408, 409, 411, 413, 414, 417, 418, 419, 424, 428, 434, 435, 443, 450, 459, 460, 463, 464, 465, 468, or any combination thereof of M. genitalium protein M (SEQ ID NO:1) or the equivalent residues of M. pneumoniae protein M (SEQ ID NO:23). In some embodiments, the one or mutations is a mutation listed in Table 4 or any combination thereof. In some embodiments, the one or mutations are at residue 83, 84, 85, 86, 87, 88, 89, 90, 91, 92, 93, 94, 95, 96, 97, 98, 99, 101, 102, 103, 105, 106, 109, 113, 116, 117, 118, 120, 126, 127, 128, 129, 130, 131, 132, 133, 134, 135, 136, 137, 138, 139, 140, 141, 142, 143, 144, 145, 146, 147, 148, 150, 151, 152, 153, 154, 155, 156, 157, 158, 159, 160, 161, 162, 164, 169, 170, 171, 172, 173, 174, 175, 176, 177, 178, 179, 180, 181, 187, 188, 189, 190, 191, 194, 195, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 339, 340, 341, 342, 343, 344, 345, 346, 347, 348, 349, 350, 351, 352, 353, 355, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 400, 401, 403, 413, 414, 415, 416, 417, 418, 419, 420, 421, 422, 423, 424, 425, 426, 427, 428, 429, 430, 431, 432, 433, 434, 435, 436, 437, 438, 439, 440, 441, 444, 446, 447, 448, 449, 450, 451, 452, 453, 459, 466, 467, 470, 471, 474, 475, 476, 477, 479, 480, 481, 482, 483, 484, or any combination thereof of M. pneumoniae protein M (SEQ ID NO:23).

In some embodiments, one or mutations are at residue 83, 90, 92, 94, 137, 142, 147, 150, 156, 184, 196, 198, 205, 211, 215, 225, 231, 232, 234, 235, 236, 237, 239, 243, 245, 250, 255, 256, 259, 264, 272, 274, 275, 276, 279, 282, 297, 300, 302, 310, 320, 326, 331, 332, 335, 342, 343, 347, 348, 355, 357, 361, 371, 374, 378, 385, 401, 402, 409, 413, 424, 460, 463, 464, 468, or any combination thereof of M. genitalium protein M (SEQ ID NO:1) or the equivalent residues of M. pneumoniae protein M (SEQ ID NO:23). In some embodiments, the one or mutations is a mutation listed in Table 5 or any combination thereof.

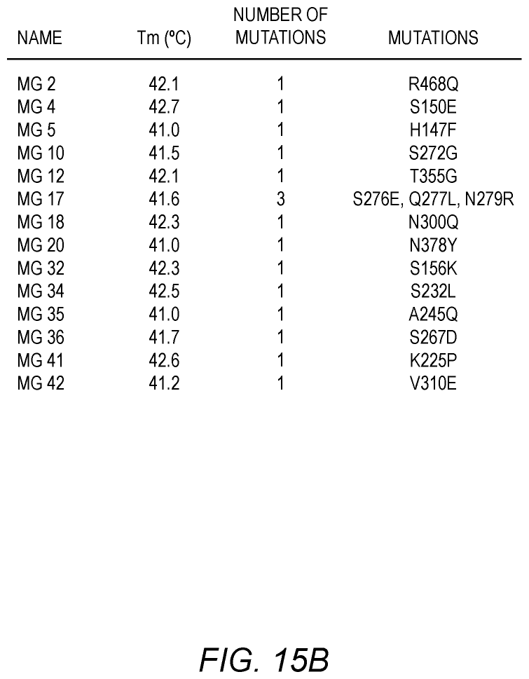

The present inventors have prepared and tested numerous mutations from the predicted residue lists alone or in combination with a high success rate of increased thermostability (stabilizing mutations) or at least maintained thermostability (neutral mutations). See FIG. 23, which shows that 79% of point mutations tested were stabilizing or neutral. The data further show that combining point mutations that increase Tm tends to produce modified protein M having even higher Tins (see FIG. 15A).

Thus, in some embodiments, the one or more mutations are at a residue that has been demonstrated to increase Tm either alone or in combination with other mutations, e.g., wherein the one or mutations are at residue 150, 196, 198, 201, 205, 224, 232, 237, 274, 282, 342, 355, 373, 400, 402, 407, 409, 413, 135, or any combination thereof of M. genitalium protein M (SEQ ID NO:1) or the equivalent residues of M. pneumoniae protein M (SEQ ID NO:23).

In some embodiments, the one or more mutations are at residues selected from the following residues or combinations of residues:
a) 237 (MG1);
b) 232 (MG8);
c) 282 (MG13);
d) 150, 196, 198, 400, 402, 407, 409 (MG15);
e) 413, 435 (MG21);
f) 373, 400 (MG22);
g) 402, 407, 409, 413 (MG23);
h) 342 (MG24);
i) 150, 196, 198, 232, 237, 282, 342, 373, 400, 402, 407, 409, 413, 435 (MG27);
j) 274 (MG28);
k) 150, 196, 198, 232, 237, 342, 400, 402, 407, 409 (MG29);
l) 373, 413, 435 (MG31)
m) 205 (MG33);
n) 355 (MG38, MG40);
o) 150, 196, 198, 342, 373, 400, 402, 407, 409 (MG43);
p) 150, 196, 198, 232, 237, 342, 373, 400, 402, 407, 409 (MG44);
q) 201, 224 (MG45);
r) 150, 196, 198, 201, 224, 232, 237, 342, 400, 402, 407, 409 (MG46);
s) 150, 196, 198, 232, 237, 342, 390, 400, 402, 407, 409, 444 (MG47);
t) 150, 196, 198, 201, 205, 224, 232, 237, 274, 342, 355, 400, 402, 407, 409 (MG48); or
u) 150, 196, 198, 232, 237, 342, 391, 400, 402, 407, 409 (MG49)
   of M. genitalium protein M (SEQ ID NO:1) or the equivalent residues of M. pneumoniae protein M (SEQ ID NO:23).

In some embodiments, the one or mutations are selected from:
a) F237T (MG1);
b) S232Q (MG8);
c) Q282D (MG13);
d) S150E, S196R, S198P, V400I, N402I, K407P, S409V (MG15);
e) L413I, T435I (MG21);
f) V373I, V400I (MG22);
g) N402L, K407P, S409V, L413I (MG23);
h) A342V (MG24);
i) S150E, S196R, S198P, S232Q, F237T, Q282D, A342V, V373I, V400I, N402I, K407P, S409V, L413I, T435I (MG27);
j) N274D (MG28);
k) S150E, S196R, S198P, S232Q, F237T, A342V, V400I, N402I, K407P, S409V (MG29);
l) V373I, L413I, T435I (MG31)
m) A205P (MG33);
n) T355D (MG38);
o) T355P (MG40);
p) S150E, S196R, S198P, A342V, V373I, V400I, N402I, K407P, S409V (MG43);
q) 150, 196, 198, 232, 237, 342, 373, 400, 402, 407, 409 (MG44);
r) S201C, A224C (MG45);
s) S150E, S196R, S198P, S201C, A224C, S232Q, F237T, A342V, V400I, N402I, K407P, S409V (MG46)
t) S150E, S196R, S198P, S232Q, F237T, A342V, F390E, V400I, N402I, K407P, S409V Y444K (MG47);
u) S150E, S196R, S198P, S201C, A205P, A224C, S232Q, F237T, N274D, A342V, T355P, V400I, N402I, K407P, S409V (MG48); or
v) S150E, S196R, S198P, S232Q, F237T, A342V, A391P, V400I, N402I, K407P, S409V (MG49)
   of M. genitalium protein M (SEQ ID NO:1) or the equivalent residues of M. pneumoniae protein M (SEQ ID NO:23).

In some embodiments, the one or more mutations are at a residue that has been demonstrated to maintain Tm either alone or in combination with other mutations, e.g., wherein the one or mutations are at residue 147, 150, 156, 225, 232, 245, 272, 276, 277, 279, 300, 310, 355, 378, 468, or any combination thereof of M. genitalium protein M (SEQ ID NO:1) or the equivalent residues of M. pneumoniae protein M (SEQ ID NO:23).

In some embodiments, the one or more mutations are at residues selected from the following residues or combinations of residues:
a) 468 (MG2);
b) 150 (MG4);
c) 147 (MG5);
d) 272 (MG10);
e) 355 (MG12);
f) 276,277,279 (MG17);
g) 300 (MG18);
h) 378 (MG20);
i) 156 (MG32);
j) 232 (MG34);
k) 245 (MG35);
l) 276 (MG36)
m) 225 (MG41); or
n) 310 (MG42)
   of M. genitalium protein M (SEQ ID NO:1) or the equivalent residues of M. pneumoniae protein M (SEQ ID NO:23).

In some embodiments, the one or mutations are selected from:
a) R468Q (MG2);
b) S150E (MG4);
c) H147F (MG5);
d) S272G (MG10);
e) T355G (MG12);
f) S276E,Q277L,N279R (MG17);
g) N300Q (MG18);
h) N378Y (MG20);
i) S156K (MG32);
j) S232L (MG34);
k) A245Q (MG35);
l) S276D (MG36)
m) K225P (MG41); or
n) V310E (MG42)
   of M. genitalium protein M (SEQ ID NO:1) or the equivalent residues of M. pneumoniae protein M (SEQ ID NO:23).

In some embodiments, the one or mutations are selected from:
a) A77E;
b) K125R;
c) V165Q;
d) H170T; or
e) A280V
   of M. pneumoniae protein M (SEQ ID NO:23).

In some embodiments, the one or mutations are selected from:
a) 159;
b) 182;
c) 102;
d) 161;
e) 86;
f) 101;
g) 147;
h) 236;
i) 348;
j) 424;
k) 147;
l) 321;
m) 389;
n) 442;
o) 119;
p) 179;
q) 186; or
r) 103
   of M. genitalium protein M (SEQ ID NO:1) or the equivalent residues of M. pneumoniae protein M (SEQ ID NO:23) or any combination thereof.

In some embodiments, the one or mutations are selected from:
a) Q159C and A182C;
b) A102C and T161C; or
c) 186C and F101C
   of M. genitalium protein M (SEQ ID NO:1) or the equivalent residues of M. pneumoniae protein M (SEQ ID NO:23) or any combination thereof, wherein the modified residues in a), b), or c) are capable of forming a di-sulfide bond between the modified residues.

In some embodiments, the one or mutations are selected from:
a) H147Y;
b) H236Y;
c) H348Y;
d) H424Y; or
e) H147Q
   of M. genitalium protein M (SEQ ID NO:1) or the equivalent residues of M. pneumoniae protein M (SEQ ID NO:23) or any combination thereof, wherein the mutation can prevent histidine protonation at that site at low pH and thereby prevent protein destabilization.

In some embodiments, the one or mutations are selected from:
a) N321H;
b) Y389H;
c) N442H;
d) P119H;
e) T179H;
f) G186H; or
g) N103H
   of M. genitalium protein M (SEQ ID NO:1) or the equivalent residues of M. pneumoniae protein M (SEQ ID NO:23) or any combination thereof, wherein the mutation can increase pH sensitivity thereby improving elution conditions for antibody purification.

In some embodiments, the one or mutations are at residues 155, 203, 243, 248, and 358 of M. pneumoniae protein M (SEQ ID NO:23).

In some embodiments, the one or mutations are A155E, K203R, H243T, V248Q, and A358V of M. pneumoniae protein M (SEQ ID NO:23).

In some embodiments, the mutation is at residue 338 of M. genitalium protein M (SEQ ID NO:1) (e.g., Y388N).

The modified Mycoplasma protein M or a functional fragment thereof may contain additional modifications beyond mutations in the amino acid sequence. In some embodiments, one or more glycosylation sites in the protein M sequence are removed, e.g., 1, 2, or 3 glycosylation sites. Three N-glycosylation sites are predicted n M. genitalium protein M based on both sequence and structural analysis with the NGlycPred server. These include N177, N213, and N274. Two O-glycosylation sites are predicted in M. genitalium protein M based on structural analysis with the NetOGlyc 4.0 server. These include T110 and T206. Suitable mutations include, without limitation, N177D, T215Y, N274D, S1121, and T206Y in any combination. In some embodiments, one or more glycosylation sites are added to the modified Mycoplasma protein M or a functional fragment thereof. Changes in glycosylation patterns may add to the thermostability of the protein and/or alter the immunogenicity of the protein by blocking antibody recognition.

For expression and purification purposes, the modified Mycoplasma protein M or a functional fragment may comprise a secretion peptide, e.g., at the N-terminus, so that the expressed protein may be secreted from the cell in which it is expressed and collected from the culture medium. Suitable secretion peptides include, without limitation, those from human serum albumin, interleukin-2, CD5, immunoglobulin Kappa light chain, trypsinogen, or prolactin for mammalian cells and Sec or Tat for bacterial cells. The secretion peptide may or may not be removed from the protein M before it is used in the methods of the invention.

In some embodiments, the modified Mycoplasma protein M or a functional fragment may comprise one or more additional mutations that alter one or more biological functions or physical characteristics of the protein. In some embodiments, the modified Mycoplasma protein M or a functional fragment may comprise one or more additional mutations that alter the affinity of the protein for antibodies. The present inventors have used computational analysis to identify residues in protein M that are predicted to increase the affinity of the protein for antibodies when mutated. Thus, in some embodiments, the one or mutations are at residue 95, 102, 103, 106, 107, 114, 116, 160, 161, 162, 163, 181, 186, 321, 381, 384, 389, 390, 391, 396, 397, 426, 429, 436, 438, 439, 441, 442, 447, 448, 449, 452, 453, 455, 456, 462, or 466, or any combination thereof of M. genitalium protein M (SEQ ID NO:1) or the equivalent residues of M. pneumoniae protein M (SEQ ID NO:23). In some embodiments, the one or mutations is a mutation listed in Table 6 or any combination thereof. In some embodiments, the one or mutations are at residue 100, 104, 107, 108, 110, 111, 112, 114, 115, 119, 121, 122, 123, 124, 125, 149, 163, 165, 166, 167, 168, 182, 183, 184, 185, 186, 192, 193, 196, 337, 338, 354, 356, 357, 399, 402, 404, 405, 406, 407,408, 409, 410, 411, 412, 442, 443, 445, 454, 455, 456, 457, 458, 460, 461, 462, 463, 464, 465, 468, 469, 472, 473, 478, or any combination thereof of M. pneumoniae protein M (SEQ ID NO:23).

In some embodiments, the modified Mycoplasma protein M or a functional fragment may comprise one or more additional mutations that alter the affinity of the protein for antibodies by modifying the pH sensitivity of the protein. The present inventors have used computational analysis to identify residues in protein M that are predicted to increase the affinity for antibodies by modifying pH sensitivity when mutated. These mutants may be particularly useful for antibody isolation due to the ability to use change in pH for elution. Thus, in some embodiments, the one or mutations are at residue 95, 103, 116, 186, 321, 389, 429, 442, or 466, or any combination thereof of M. genitalium protein M (SEQ ID NO:1) or the equivalent residues of M. pneumoniae protein M (SEQ ID NO:23). In some embodiments, the one or mutations is a mutation listed in Table 7 or any combination thereof.

In some embodiments, the modified Mycoplasma protein M or a functional fragment may comprise one or more additional mutations that reduce or eliminate affinity for antibodies. Examples include, without limitation, mutations at residues 390 and 444, e.g., 390E and Y444K of M. genitalium protein M (SEQ ID NO:1) or the equivalent residues of M. pneumoniae protein M (SEQ ID NO:23).

In some embodiments, the modified Mycoplasma protein M or a functional fragment may comprise a deletion from about residue 185 to about residue 342 of M. genitalium protein M (SEQ ID NO:1) or the equivalent residues of another Mycoplasma protein M. In some embodiments, the deleted amino acids are replaced by a short linker. In some embodiments, the deletion creates newly exposed hydrophobic residues and these newly exposed residues have one or more mutations.

In one aspect of the invention, a fusion protein comprises a modified Mycoplasma protein M or a functional fragment thereof, a linker, and a peptide. In some embodiments, the fusion protein has a modified Mycoplasma protein M or a functional fragment thereof with one or more mutations that enhance the affinity or avidity of the modified Mycoplasma protein M or a functional fragment thereof to antibodies. In some embodiments, the peptide is a dimerizing peptide (e.g., IL-GCN4, C-IL-GCN4). In other embodiments, the peptide is a Fab binding polypeptide or an Fc binding polypeptide. In some embodiments, the Fab binding polypeptide is Protein L, protein A, or protein G. In some embodiments, the Fab binding polypeptide binds to the Fc portion of immunoglobulins and/or decreases Fc receptor recycling of the immunoglobulins and/or increases binding of the fusion protein to immunoglobulins. In other embodiments, the peptide comprises a dimerizing peptide and Protein L. In one embodiment, the fusion protein, starting from the N-terminus, comprises in order a dimerizing peptide, a first linker, Protein L, a second linker, and a modified Mycoplasma protein M or a functional fragment thereof.

In other embodiments, the peptide is a Fc binding polypeptide, which can be a Fc receptor polypeptide. In some embodiments, the peptide comprises a Fc binding polypeptide and Protein L. In one embodiment, the fusion protein, starting from the N-terminus, comprises in order a Fc binding polypeptide, a first linker, Protein L, a second linker, and the modified Mycoplasma protein M or a functional fragment thereof. In some embodiments, the peptide is a Fc domain, which may include complement binding residues for binding to a Fc receptor polypeptide and/or one or more mutations of complement binding residues for binding to a Fc receptor polypeptide. In some embodiments, the peptide is a Fc receptor protein and the fusion protein decreases the cellular uptake of antibodies, decreases antibody recycling and causes immunoglobulin depletion by decreasing antibody recycling, and/or increases affinity of the fusion protein for immunoglobulins. In other embodiments, the fusion protein comprises two or more modified Mycoplasma protein M or a functional fragment thereof. In other embodiments, the fusion protein comprises half-life extension moieties, such as human albumin or addition of polyethylene glycol (PEG), protein modifications such as acylation, methylation, phosphorylation, sulfation, farnesylation, ubiquitination, site-selective conjugation, glycosylation, introduction of PTMs, PEGylation, ligation, polymerization of protein-based initiators, or any modifications that may alter binding, affinity, binding targets, or any combination thereof.

The protein M proteins according to the invention are produced and characterized by methods well known in the art and as described herein, such as recombinant expression.

An additional aspect of the invention provides an isolated polynucleotide encoding the protein M or a functional fragment or derivative thereof of this invention and an expression cassette for producing the protein M or a functional fragment or derivative thereof.

The polynucleotide may be operably linked to regulatory elements to aid in expression of the protein. In some embodiments, the polynucleotide is operably linked to a promoter. The promoter may be a bacterial promoter (e.g., operable in E. coli) or a mammalian promoter (e.g., a human promoter).

In some embodiments, the polynucleotide may be codon optimized to enhance expression of the protein in a host cell. In one embodiment, the polynucleotide is codon optimized for expression in bacteria, such as E. coli. In another embodiment, the polynucleotide is codon optimized for expression in a mammalian cell, such as a human cell. One example is the sequence of SEQ ID NO:26, which is a codon optimized for expression in human cells, or a sequence at least 90%, 91%, 92%, 93%, 94%, 95%, 96%, 97%, 98%, 99%, or 99.5% identical thereto. In a further embodiment, the polynucleotide is codon optimized for expression in both bacteria, such as E. coli, and a mammalian cell, such as a human cell. One example is the sequence of SEQ ID NO:25, which is a codon optimized for expression in both E. coli and human cells, or a sequence at least 90%, 91%, 92%, 93%, 94%, 95%, 96%, 97%, 98%, 99%, or 99.5% identical thereto.

Another aspect of the invention is a vector, e.g., an expression vector, comprising the polynucleotide of the invention. The vector may be any type of vector known in the art, including, without limitation, plasmid vectors and viral vectors. The vector may be, for example, a bacterial vector (e.g., an E. coli vector) or a mammalian cell vector (e.g., a human cell vector).

A further aspect of the invention relates to a cell comprising the polynucleotide and/or vector of the invention (e.g., an isolated cell, a transformed cell, a recombinant cell, etc.). Thus, various embodiments of the invention are directed to recombinant host cells containing the vector (e.g., expression cassette). Such a cell can be an isolated cell. In some embodiments, the polynucleotide is stably incorporated into the genome of the cell. In some embodiments, the cell may be a bacterial cell, such as E. coli, or a mammalian cell, such as a human cell.

A further aspect of the invention relates to a kit comprising the modified Mycoplasma protein M or a functional fragment thereof, polynucleotide, vector, and/or transformed cell of the invention. The kit may include additional reagents for carrying out one of the methods described herein. The reagents may be included in suitable packages or containers. Additional reagents include, without limitation, buffers, labels, enzymes, detection reagents, etc.

When a kit is supplied, the different components may be packaged in separate containers and admixed immediately before use. Such packaging of the components separately may permit long-term storage without losing the active components' functions. Kits may also be supplied with instructional materials. Instructions may be printed on paper or other substrate, and/or may be supplied as an electronic-readable medium.

### Heterologous Agents

As described above, the heterologous agent may be one for which neutralizing antibodies are present in the subject prior to administration of the heterologous agent or one that is likely to raise neutralizing antibodies upon administration to the subject. In some embodiments, the heterologous agent may be a nucleic acid delivery vector (e.g., a viral vector or a non-viral vector), a gene editing complex (e.g., a CRISPR complex), a protein, or a nucleic acid.

Any nucleic acid sequence(s) of interest may be delivered in the nucleic acid delivery vectors of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor −3 and −4, brain-derived neurotrophic factor, bone morphogenic proteins [including RANKL and VEGF], glial derived growth factor, transforming growth factor −α and −β, and the like), lysosomal acid α-glucosidase, α-galactosidase A, receptors (e.g., the tumor necrosis growth factor α soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the nucleic acid may encode a functional nucleic acid, i.e., nucleic acid that functions without getting translated into a protein, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan [e.g., α, β, γ], RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The nucleic acid delivery vectors may also comprise a nucleic acid that shares homology with and recombines with a locus on a host chromosome. This approach can be utilized, for example, to correct a genetic defect in the host cell.

The present invention also provides nucleic acid delivery vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a nucleic acid introduced into a recombinant vector genome. Any immunogen of interest as described herein and/or as is known in the art can be provided by the nucleic acid delivery vectors.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The nucleic acid delivery vectors provide a means for delivering nucleic acids into a broad range of cells, including dividing and non-dividing cells. The nucleic acid delivery vectors can be employed to deliver a nucleic acid of interest to a cell in vitro, e.g., for ex vivo gene therapy. The nucleic acid delivery vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The nucleic acid delivery vectors can also be used to produce a polypeptide of interest or functional RNA in a subject (e.g., using the subject as a bioreactor to produce the polypeptide or to observe the effects of the functional nucleic acid on the subject, for example, in connection with screening methods).

In general, the nucleic acid delivery vectors of the present invention can be employed to deliver a nucleic acid encoding a polypeptide or functional nucleic acid to treat and/or prevent any disease state for which it is beneficial to deliver a therapeutic polypeptide or functional nucleic acid. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used prior to and/or following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, nucleic acid delivery vectors permit the treatment and/or prevention of genetic diseases.

The nucleic acid delivery vectors may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Nucleic acid delivery vectors find use in diagnostic and screening methods, whereby a nucleic acid of interest is transiently or stably expressed in a transgenic animal model.

The nucleic acid delivery vectors can also be used for various non-therapeutic purposes, including but not limited to use in protocols to assess gene targeting, clearance, transcription, translation, etc., as would be apparent to one skilled in the art. The nucleic acid delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the nucleic acid delivery vectors of the present invention may be used to produce an immune response in a subject. According to this embodiment, a nucleic acid delivery vectors comprising a nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the nucleic acid delivery vectors may be administered to a cell ex vivo and the altered cell is administered to the subject. The nucleic acid delivery vectors comprising the nucleic acid is introduced into the cell, and the cell is administered to the subject, where the nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the nucleic acid delivery vector or cell comprising the nucleic acid can be administered in an immunogenically effective amount, as described below.

The nucleic acid delivery vectors can also be administered for cancer immunotherapy by administration of a nucleic acid delivery vector expressing one or more cancer cell antigens (or an immunologically similar molecule) or any other immunogen that produces an immune response against a cancer cell. To illustrate, an immune response can be produced against a cancer cell antigen in a subject by administering a nucleic acid delivery vectors comprising a nucleic acid encoding the cancer cell antigen, for example to treat a patient with cancer and/or to prevent cancer from developing in the subject. The nucleic acid delivery vectors may be administered to a subject in vivo or by using ex vivo methods, as described herein. Alternatively, the cancer antigen can be expressed as part of the nucleic acid delivery vectors.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a nucleic acid delivery vectors. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polynucleotide including those described herein. As a further option, the subject can be a laboratory animal and/or an animal model of disease. Preferably, the subject is a human.

In certain embodiments, the heterologous agent and protein M or a functional fragment or derivative thereof is administered to a subject in need thereof as early as possible in the life of the subject, e.g., as soon as the subject is diagnosed with a disease or disorder. In some embodiments, the method are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

In particular embodiments, the present invention provides one or more pharmaceutical compositions comprising protein M or a functional fragment or derivative thereof, alone or together with a heterologous agent, in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro, e.g., as part of an ex vivo method. The heterologous agent (e.g., nucleic acid delivery vector, e.g., viral vector) may be introduced into the cells at the appropriate amount, e.g., multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 103 infectious units, more preferably at least about 10⁵ infectious units are introduced to the cell.

The cell(s) into which the nucleic acid delivery vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The nucleic acid delivery vectors can be introduced into cells in vitro for the purpose of administering the modified cell to a subject. In particular embodiments, the cells have been removed from a subject, the nucleic acid delivery vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the nucleic acid delivery vectors can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10′ to about 10⁶ cells will be administered per dose in a pharmaceutically acceptable carrier. In particular embodiments, the cells transduced with the nucleic acid delivery vector are administered to the subject in a treatment effective or prevention effective amount in combination with a pharmaceutical carrier.

In some embodiments, the nucleic acid delivery vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the heterologous agent (e.g., nucleic acid delivery vector) to subjects. Administration of the nucleic acid delivery vectors to a human subject or an animal in need thereof can be by any means known in the art. Optionally, the nucleic acid delivery vector is delivered in a treatment effective or prevention effective dose in a pharmaceutically acceptable carrier.

The nucleic acid delivery vectors can further be administered to elicit an immunogenic response (e.g., as a vaccine). Typically, immunogenic compositions of the present invention comprise an immunogenically effective amount of nucleic acid delivery vector in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the nucleic acid delivery vector (e.g., viral vector) to be administered to a subject depend upon the mode of administration, the disease or condition to be treated and/or prevented, the individual subject's condition, the particular nucleic acid delivery vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸-10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator intermus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronatorteres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, stemohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The heterologous agent can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the heterologous agent is administered to a limb (arm and/or leg) of a subject (e.g., a subject with muscular dystrophy such as DMD) by limb perfusion, optionally isolated limb perfusion (e.g., by intravenous or intra-articular administration. In embodiments of the invention, the heterologous agent can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the heterologous agent can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The heterologous agent can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the heterologous agent. In representative embodiments, a depot comprising the heterologous agent is implanted into skeletal, smooth, cardiac and/or diaphragm muscle tissue or the tissue can be contacted with a film or other matrix comprising the heterologous agent. Such implantable matrices or substrates are described in U.S. Pat. No. 7,201,898.

In particular embodiments, a heterologous agent is administered to skeletal muscle, diaphragm muscle and/or cardiac muscle (e.g., to treat and/or prevent muscular dystrophy or heart disease [for example, PAD or congestive heart failure]).

In representative embodiments, the invention is used to treat and/or prevent disorders of skeletal, cardiac and/or diaphragm muscle.

In a representative embodiment, the invention provides a method of treating and/or preventing muscular dystrophy in a subject in need thereof, the method comprising: administering a treatment or prevention effective amount of a heterologous agent to a mammalian subject, wherein the heterologous agent comprises a nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the heterologous agent can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional nuclei acid (e.g., functional RNA, e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid α glucosidase]). Other suitable proteins for treating and/or preventing metabolic disorders are described above. The use of muscle as a platform to express a nucleic acid of interest is described in U.S. Patent Publication No. 2002/0192189.

Thus, as one aspect, the invention further encompasses a method of treating and/or preventing a metabolic disorder in a subject in need thereof, the method comprising: administering a treatment or prevention effective amount of a heterologous agent to a subject (e.g., to skeletal muscle of a subject), wherein the heterologous agent comprises a nucleic acid encoding a polypeptide, wherein the metabolic disorder is a result of a deficiency and/or defect in the polypeptide. Illustrative metabolic disorders and nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering heterologous agent to skeletal muscle are described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of a heterologous agent of the invention to a mammalian subject, wherein the heterologous agent comprises a nucleic acid encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206 and/or mir-208.

Injectables can be prepared in conventional forms, either as liquid solutions or suspensions, solid forms suitable for solution or suspension in liquid prior to injection, or as emulsions. Alternatively, one may administer the heterologous agent in a local rather than systemic manner, for example, in a depot or sustained-release formulation. Further, the heterologous agent can be delivered adhered to a surgically implantable matrix (e.g., as described in U.S. Patent Publication No. 2004-0013645).

The heterologous agent disclosed herein can be administered to the lungs of a subject by any suitable means, optionally by administering an aerosol suspension of respirable particles comprised of the heterologous agent, which the subject inhales. The respirable particles can be liquid or solid. Aerosols of liquid particles comprising the heterologous agent may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the heterologous agent may likewise be produced with any solid particulate medicament aerosol generator, by techniques known in the pharmaceutical art.

The heterologous agent can be administered to tissues of the CNS (e.g., brain, eye) and may advantageously result in broader distribution of the heterologous agent than would be observed in the absence of the present invention.

In particular embodiments, the heterologous agent may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The heterologous agent of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a heterologous agent encoding one or more neurotrophic factors.

Age-related macular degeneration involves both angiogenesis and retinal degeneration. This disorder can be treated by administering a heterologous agent encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the heterologous agent. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a heterologous agent of the invention to treat a pituitary tumor. According to this embodiment, the heterologous agent encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the heterologous agent can comprise a secretory signal as described in U.S. Pat. No. 7,071,172.

In representative embodiments of the invention, the heterologous agent is administered to the CNS (e.g., to the brain or to the eye). The heterologous agent may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The heterologous agent may also be administered to different regions of the eye such as the retina, cornea and/or optic nerve.

The heterologous agent may be delivered into the cerebrospinal fluid (e.g., by lumbar puncture) for more disperse administration of the heterologous agent. The heterologous agent may further be administered intravascularly to the CNS in situations in which the blood-brain barrier has been perturbed (e.g., brain tumor or cerebral infarct).

The heterologous agent can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the heterologous agent is administered in a liquid formulation by direct injection (e.g., stereotactic injection) to the desired region or compartment in the CNS. In other embodiments, the heterologous agent may be provided by topical application to the desired region or by intra-nasal administration of an aerosol formulation. Administration to the eye, may be by topical application of liquid droplets. As a further alternative, the heterologous agent may be administered as a solid, slow-release formulation (see, e.g., U.S. Pat. No. 7,201,898).

In yet additional embodiments, the heterologous agent can used for retrograde transport to treat and/or prevent diseases and disorders involving motor neurons (e.g., amyotrophic lateral sclerosis (ALS); spinal muscular atrophy (SMA), etc.). For example, the heterologous agent can be delivered to muscle tissue from which it can migrate into neurons.

The protein M or a functional fragment or derivative thereof may be administered by any of the routes or schedules described above for the heterologous agent The protein M or a functional fragment or derivative thereof may be administered by a different route or schedule than the heterologous agent.

Having described the present invention, the same will be explained in greater detail in the following examples, which are included herein for illustration purposes only, and which are not intended to be limiting to the invention.

### EXAMPLES

### Example 1: Methods

AAV virus production: AAV vectors were produced using a standard approach with three-plasmid transfection in HEK293 cells. Briefly, the AAV transgene plasmid pTR-CBA-Luc was co-transfected with an AAV Rep/Cap helper plasmid (pXR2 or pXR8) and adenovirus helper plasmid pXX6-80. 72 hours later, cell cultures were harvested and lysed by freeze thaw and ultra sonication. Clarified cell lysate was DNAse treated and ultra-centrifuged in a 15%/25%/40%/60% iodixanol step gradient and purified by anion exchange Q-column. Purified AAV vector was tittered by qPCR with primers directed to amplify a segment of the packaged AAV transgene.

Protein Production: The plasmid pET-28b(+) which encodes the protein M, a truncated M. genitalium Protein MG281 lacking the transmembrane domain (amino acids 74 to 479) and carries a N-terminal His-Tag and a thrombin cleavage site, was generously provided by Rajesh. Plasmids were propagated in electro-competent DH10B cells and purified using a PureLink Maxi-Prep kit from Invitrogen. The pET-28b(+) plasmid was transiently transfected into BL21/DE3 cells using an overnight starter culture. Auto-induction media (Magic media from Invitrogen) was seeded with the starter culture and grown at 18° C. for 3 days in 1 L to 4 L culture volumes. The culture was then pelleted by centrifugation and frozen down at −80° C.

Protein Purification: The frozen bacterial cell culture pellet was thawed, lysed by sonication, DNAse treated, and clarified by centrifugation. Clarified bacterial lysate was dialyzed into nickel-binding buffer (20 mM imidazole, 50 mM sodium phosphate pH 7.4, 500 mM NaCl, 0.02% sodium azide) and passed through a nickel His-trap FF column using FPLC. Protein M bound by the nickel column was then eluted by the same buffer base but containing 500 mM imidazole. Protein M was then run through an 5-100 size exclusion column, dialyzed into phosphate buffered saline with 2% glycerol, and quantified using spectrophotometry. Protein M identity was confirmed using SDS-PAGE protein gel electrophoresis for protein separation followed by a Coomassie blue protein stain to correctly identify the 48 kDa Protein M band.

Western Blot: After protein separation on an 8% SDS-PAGE gel, the proteins were transferred onto a PVDF membrane. Immunoblotting was performed in 5% non-fat milk using anti-His primary antibody 1:1000 dilution (10 μg/ml). The secondary goat anti-human IgG antibody was conjugated to horseradish peroxidase (1:10,000 dilution).

Cell Culture: HEK-293 cells and Huh7 cells were used for all in vitro AAV neutralization experiments and for enhancement of transduction, respectively. Cells were maintained at 37° C. in 5% CO₂ with Dulbecco's modified Eagle's medium supplemented with 10% bovine calf serum and penicillin-streptomycin.

Human IVIG and immunized mouse serum: 10% Human IVIG (Gamunex) was purchased from Grifols Therapeutics Inc. (Research Triangle Park, NC, USA). Serum was collected and pooled from 12 different mice (50% male, 50% female) after IP administration of 3×10¹′ viral genomes of AAV8-FVIII followed by a boost administration of the same vector 2 weeks later, and a second boost 6 weeks after the first administration. Human IVIG and mouse serum were aliquoted and stored at −80° C. for future use.

In vitro AAV Neutralization Assay: NAb analyses were performed as described previously with slight modification. Cells were pelleted by centrifugation and resuspended in serum free X-Vivo 10 medium, then plated in either a 48-well or 96-well plate format. Human IVIG or serum was serially diluted either two-fold or ten-fold. AAV-Luc was incubated with either human IVIG or mouse serum for 1 h at 4° C. before adding AAV and incubating an additional 1 h at 4° C. AAV+serum incubations were then mixed with cells suspended in serum free media at the time of plating. In the neutralization experiments using protein M, 3 different incubations scenarios were tested with each step for 1 h at 4° C.: neutralizing serum incubated first with protein M followed by AAV, AAV incubated first with neutralizing serum followed by protein M, and AAV incubated first with protein M followed by neutralizing serum. Cells were seeded in a 48-well plate in 200 μl media, or in a 96-well plate in 100 μl media. After transduction, cells were cultured for 24-48 h at 37° C. to allow for AAV-Luciferase transgene expression. To measure Luc activity, cells were lysed with passive lysis buffer (Promega, Madison, WI, USA) and luciferase signal was measured with a Wallac1420 Victor 2 automated plate reader. NAb titers were defined as the highest dilution for which luciferase activity was 50% lower than serum-free controls.

In Vivo AAV Neutralization and Passive Transfer of NAb Serum: Different amounts of serum containing AAV NAbs were injected into the retro-orbital vein of C57BL/6 mice and 20-minutes later AAV was injected. For mice receiving protein M treatment before AAV administration, protein M (either 6.3 mg for 2:1 ratio, 3.15 mg for 1:1 ratio, or 1.58 mg for 0.5:1 ratio) was delivered by retro-orbital vein 5-15 minutes after serum injection. AAV was administered 5 minutes later by systemic injection of 2×10¹² particles per kg of AAV-Luc vector. Imaging was performed 1 day after AAV administration, as well as at 1 week and 9 day time points post-injection.

### Example 2: Protein M Ablates the Inhibition Activity of IVIG on AAV Transduction

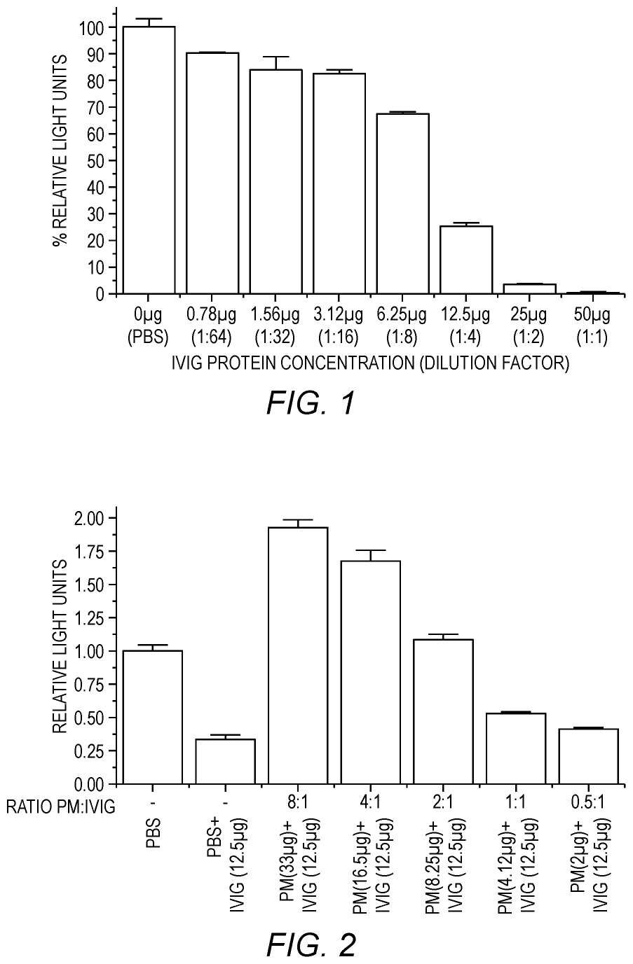

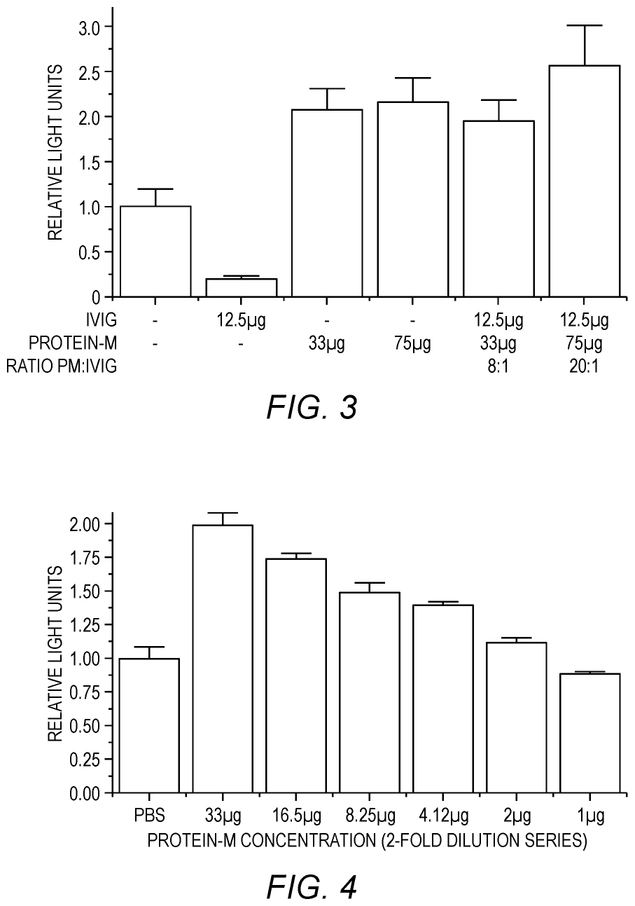

In order to study the antibody blocking function of protein M, in vitro neutralization assays of AAV by human intravenous immunoglobulin gamma (IVIG) were set up. Serial dilutions of IVIG were used to perform an AAV neutralization assay to determine the quantity of IVIG that will neutralize a given amount of AAV2. Luciferase activity generated by cellular transduction with AAV-Luciferase vectors served as a functional read out of gene expression. The neutralization was determined as the percent of luciferase activity normalized to no IVIG controls. It was found that 12.5 μg of IVIG neutralizes 75% (+/−5%) of AAV2 (FIG. 1) and this quantity of IVIG was chosen to move forward with experiments design to test protein M blocking of IVIG for AAV neutralization. Next, a dose dilution series was conducted of protein M (SEQ ID NO:2) which was collected by removing His-tag by thrombin cleavage and it's ability to block 12.5 μg of IVIG (FIG. 2), where protein M was incubated with IVIG for 1 h followed by AAV2 for 1 h at 4° C. before cell culture transduction. It was found that a molar ratio of 2 protein M molecules to 1 IgG molecule was sufficient to prevent neutralization to an equivalent level as the no IVIG control. Furthermore, it was found that higher molar ratios of protein M to IVIG enhanced the luciferase signal to levels greater than the no IVIG control (FIG. 2). 8 protein M molecules to 1 IgG increased luciferase expression by 2-fold. In order to see if enhancement increases further with greater molar ratios, an 8:1 and a 20:1 ratio of protein M to IVIG was tested in the same experimental conditions. It was found that increasing the protein M dose by 2.5-fold (from 8:1 to a 20:1 ratio) provided litter further enhancement since the corresponding signal increased by only 0.25-fold over the 8:1 ratio (FIG. 3). Additionally, it was found that protein M blocking activity of immunoglobulin does not depend on cleavage of the N-terminal His tag, therefore Protein M without cleavage of His tag was used for the next experiments.

### Example 3: Interaction of Protein M with AAV Vector Virions Enhances AAV Transduction

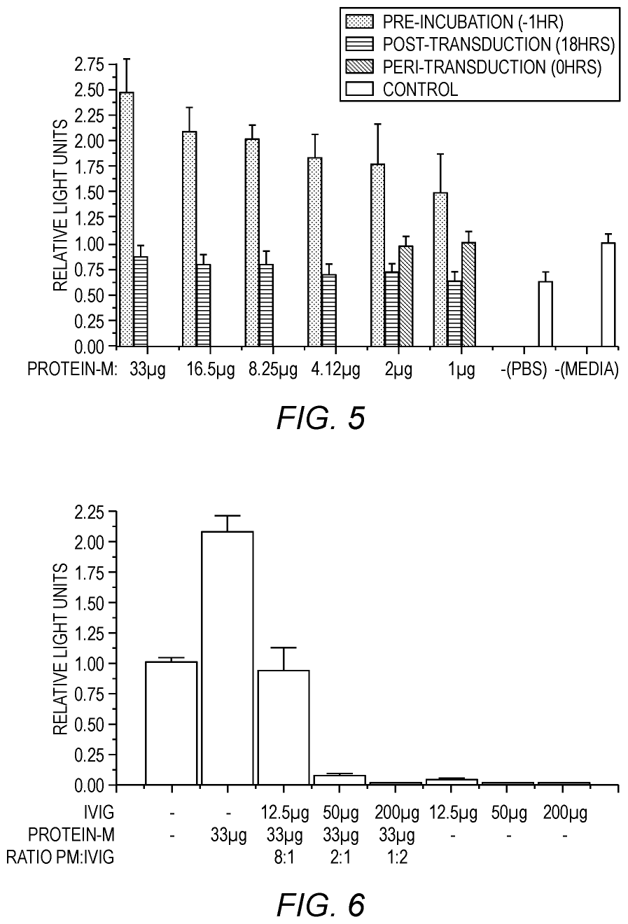

Previous studies from the inventors have demonstrated that interaction of serum proteins with AAV virions is able to enhance AAV transduction. In order to further characterize the enhancement function of protein M on AAV transduction, a dose-response assay was performed without IVIG where protein M at serial 2-fold dilution was incubated with AAV for 1 h prior to cell culture transduction. In FIG. 4 it was found that the 8:1 ratio (33 μg protein M) of protein M from the previous experiments was able to dose dependently enhance AAV transduction in the absence of IVIG, and that enhancement was lost at dilutions below 2 μg of protein M for 2×10⁸ viral particles. The equivalent molar ratio of protein M molecules to AAV particles at which enhancement was lost was below 40,000:1. Next, it was demonstrated that protein M enhancement of transduction is dependent on incubation of protein M with AAV. In FIG. 5 a cell culture transduction assay was performed where protein M was added to AAV either 1 h before adding to the cells (pre-incubation, −1 h time point), added at the time of transduction without pre-incubation (peri-transduction, 0 h time point), or 18 h after adding AAV to the cells (post-transduction, 18 h time point). It was discovered that dose-dependent enhancement was seen in the pre-incubation group similar to that in FIG. 4, but not in the other 2 groups where protein M and AAV2 were not incubated together prior to the assay. Furthermore, this result also argues that the mechanism of protein M enhancement involves interaction with the vector capsid, and not a biological effect of protein M on the cells since adding protein M to the cells at the time of transduction had no impact on luciferase signal. Finally, it was demonstrated that protein M is not able to block neutralization when IVIG is first pre-incubated with AAV2 for 1 h followed by a 1 h incubation with protein M, and that protein M enhancement of AAV2 luciferase signal is only present when AAV2 is not fully neutralized by IVIG. For this assay 12.5 μg of IVIG (which neutralizes 75-80% of AAV2), 50 μg of IVIG (which neutralizes 99% of AAV2, from FIG. 1), or 200 μg of IVIG (which neutralizes 100% of AAV2) was used. In FIG. 6 it was observed that when 99-100% of AAV2 was neutralized by IVIG (50 μg and 200 μg), then protein M is incapable of blocking neutralization and cannot provide transduction enhancement. In combination with the data in FIGS. 2 and 3, this result implicates that the excess amount of protein M after binding to immunoglobulin is able to interact with AAV virions and results in enhanced transduction.

### Example 4: Pre-Incubation of Protein M with AAV Vector Virions Protects AAV Neutralization of IVIG

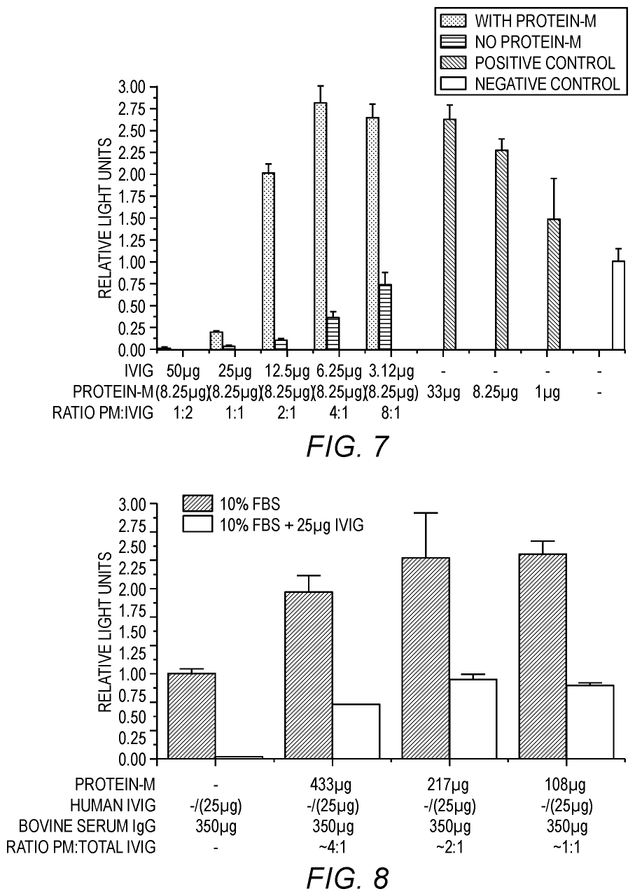

It was next decided to investigate the effect of pre-incubating protein M first with AAV and then IVIG instead of allowing protein M to first interact with IVIG. The ability of protein M to block neutralization was examined when protein M was incubated with AAV2 for 1 h followed by addition of IVIG and incubation for an additional 1 h at 4° C. before cell transduction. In FIG. 7 it was found that when protein M concentrations are keep static (8.25 μg), and the dose of IVIG is serially diluted from 50 μg to 3.12 μg (1:2 to 8:1 molar ratio of protein M to IVIG) then protection of the vector from neutralization is achieved at a 2:1 ratio or greater, which is similar to the previous results when protein M was incubated with IVIG prior to AAV addition. In an effort to simulate an environment closer to the in vivo situation, where other types of proteins are present in the serum besides IgG, a similar in vitro neutralization test was performed in the presence of 10% Fetal Bovine Serum (FBS). As per the manufacturer's IgG quantifications for the bovine serum, it was estimated the media volume contained ˜350 μg of bovine IgG per well. 25 μg IVIG or serum free media was then spiked in half the wells to neutralize AAV or serve as a control without neutralizing activity. In FIG. 8, when protein M was incubated with AAV for 1 h prior to transduction, and total IgG was added (human and bovine) at different molar ratios of 4:1, 2:1, and 1:1 (protein M to IgG), then protection from neutralization was observed even at the 1:1 ratio. These results indicate that protein M is able to bind and block immunoglobulins even in the presence of other serum proteins.

### Example 5: Efficient Blocking Function of Protein M on Neutralizing Activity of Murine Serum to AAV In Vitro

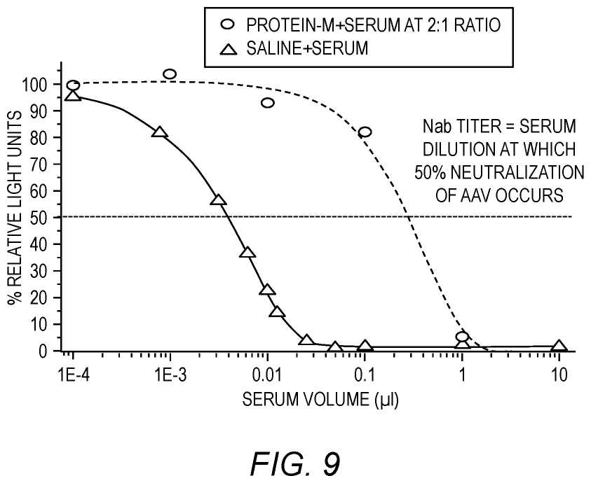

To translate the finding from IVIG to a real situation, in vitro neutralization experiments were first performed with AAV8-Luciferase vectors using serum from mice that were immunized with an AAV8 capsid vector. When both the serum from immunized mice and protein M were serially diluted to maintain a molar ratio of 2:1 the results compared to serum only controls, it was found that protein M allows escape from neutralization over an estimated ˜100-fold dilution of neutralizing serum at a titer of 1:2,564 (50% neutralization at 0.0039 μl of serum vs 0.2744 μl of serum in the presence of protein M, FIG. 9).

### Example 6: Efficient Blocking Function of Protein M on Neutralizing Activity of Murine Serum to AAV In Vivo

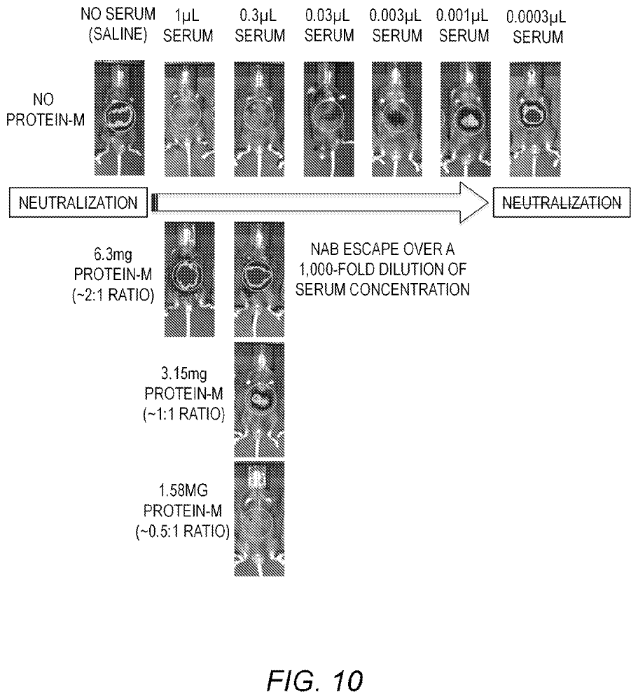

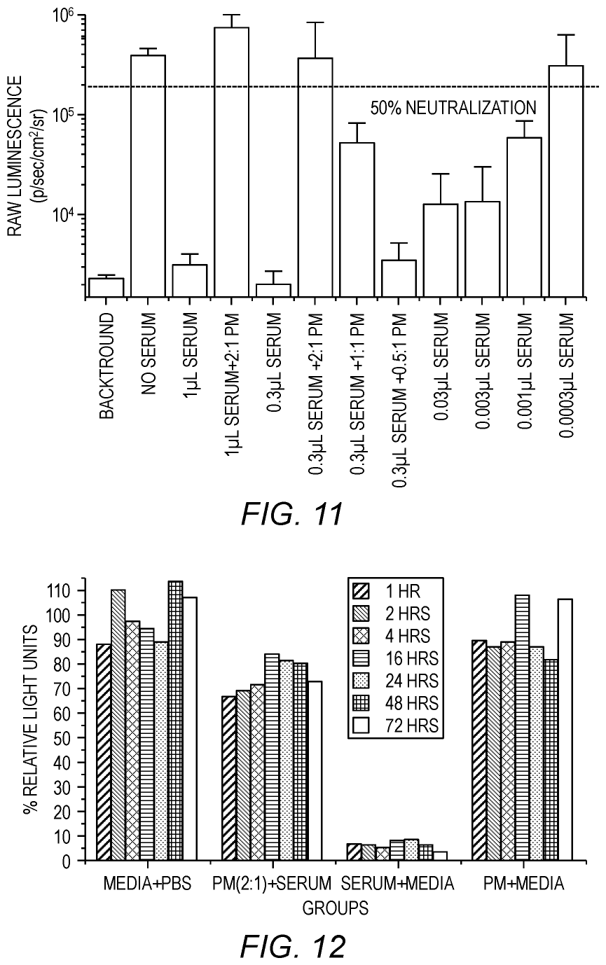

Next, in vivo experiments were carried out where different volumes of neutralizing serum were passively transferred into naïve mice via retro-orbital injection. After letting the serum perfuse through the mice for 5-15 minutes, protein M was administered systemically to the mice also via retro-orbital injection followed 5 minutes later by AAV8-Luc (2×10¹⁰ viral genomes). FIG. 10 shows that protein M, at an estimated molar ratio of 2:1 (6.3 mg) to total immunoglobulin in the mouse, is able to prevent neutralization of AAV8 by 1 μl of AAV8-NAb serum (titer 1:2,564). This is compared to an in vivo neutralization assay of serially diluted anti-AAV8 serum where greater than 50% of AAV8-Luc is neutralized at serum volumes between 1 μl to 0.001 μl, representing AAV NAb escape over a 1,000-fold difference in concentration (FIG. 11).

### Example 7: The Stability of Protein M/Immunoglobulin Complex

To study how stable the complex of protein M with immunoglobulins is, the assay was first carried out in vitro. Protein M was pre-incubated with mouse sera at molecular ratio of 2:1 at 37° C. for different duration from 1 h to 72 h, and then AAV8 vectors added for one more hour at 4° C. for neutralizing analysis. As shown in FIG. 12, the pre-formed complex between anti-AAV8 immunoglobulins in mouse serum and protein M was stable for greater than 72 h when incubated at 37° C. before being added to cell culture, as compared to controls containing neutralizing serum without protein M, or containing only PBS or media. This result suggests the formed complex between protein M and immunoglobulin is highly stable in vitro.

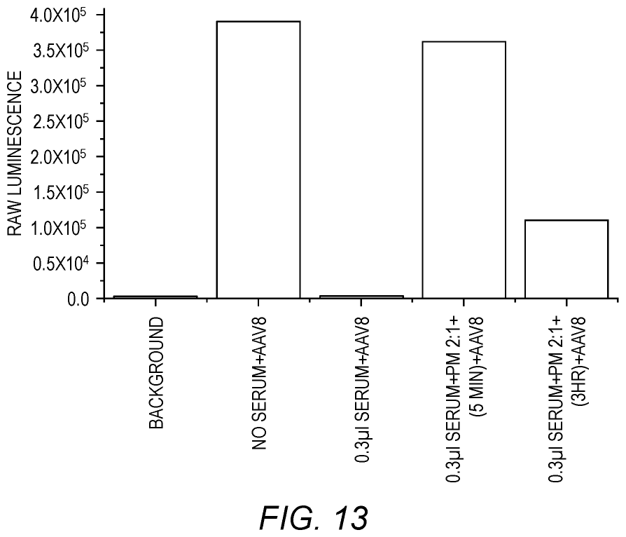

Next, in vivo stability of protein M was examined after passive transfer of 0.3 μl of AAV8 neutralizing serum. It was found that if protein M was administered and waited 3 h instead of 5 minutes, then the antibody blocking function of protein M is reduced by ˜70% from 3.5×10⁵ to 1×10⁵ photons/second/cm²/spontaneous rate (FIG. 13). This result indicates the NAb blocking effect of protein M is short lived in vivo.

Gene therapy with AAV vectors has been successful in clinical trials. However, the high prevalence of AAV neutralizing antibodies in human population prevents more patients from benefitting from this effective gene delivery. In this study, it was found that the protein M derived from Mycoplasma is able to interact with NAbs and enhance AAV transduction. The minimum dose of protein M needed to block NAb activity was 2 fold more molecules than immunoglobulin. Direct interaction of protein M with AAV virions also increased AAV transduction. Neutralizing antibody assay showed that approximate 100 fold of NAb activity was decreased when AAV immunized mouse sera were incubated with protein M in vitro, and over 1000 fold protection was observed in mice with adoptive transfer of NAb positive sera when protein M was applied. Although the complex of protein M with immunoglobulin was stable in vitro over time, the protein M gradually loses its protect function from the neutralization of immunoglobulins in vivo.

To overcome AAV NAbs, a number of strategies have been exploited in the laboratory. One approach is to mask the AAV surface for blocking Nab recognition using a polymer or exosome for coating. While promising, this approach may change the AAV transduction profile. A second approach is to use error-prone PCR or DNA shuffling to generate a library of AAV capsid variants and select NAb escape mutants in the presence of NAbs in vitro and in vivo. This approach has yielded novel capsids; however, it bears the potential limitation of generating capsids with unknown transduction efficiency in human since these mutants are isolated from and tested in animal tissues and the data from animal studies do not always translate into human as well as no authentic systems are available to predict AAV transduction in human tissues. A third approach has been to use alternative serotypes of AAV that show low or absent NAb cross-reactivity. While this popular strategy is logical and successful in animal models, concerns remain about the existence of cross-reactivity in most humans that may not be predicted in the animals. A final laboratory approach is to rationally engineer the NAb binding domains on the AAV capsid surface to eliminate the NAb binding sites. This strategy requires information about monoclonal antibody epitopes and the structure of AAV virion, and is inherently limited due to the fact that the NAbs from human sera are polyclonal and it is impossible to obtain mAbs from humans that represent all generated NAbs. Several clinical related approaches have also been studied: one example is to perform plasmapheresis prior to vector delivery. However, due to the relative inefficiency of each round of apheresis and the fact that even low titers of NAbs (<1:5) can abrogate AAV transduction, this strategy is only suitable for patients with lower starting titers of AAV NAbs and requires multiple sessions of apheresis. Similarly, the use of anti-CD20 antibody (Rituximab) can achieve B cell depletion, but it takes a long time (about 6-9 months) and is only effective in reducing AAV NAb in a minority of subjects. A final clinical approach is to use excessive empty AAV capsids as decoys for NAbs. The concern remains that the addition of empty particles increases the AAV capsid load which potentially increases capsid specific cytotoxic immune response mediated the elimination of AAV transduced cells and perhaps competes with full AAV particles for effective transduction. Additionally, empty capsids may induce a greater liver inflammation than full AAV vectors. Compared to the low efficiency of NAb protection or complications in the above approaches, in this study, a strategy with greater potential to block neutralizing antibody activity was demonstrated using immunoglobulin binding protein M. The NAb evasion ability was achieved for 100 fold in vitro and over 1000 fold in vivo when protein M was used.

Protein M functions as a universal antibody binding protein which blocks mammalian IgG, IgM, and IgA antibody classes by universally binding to conserved regions on the antibody light and heavy chains, causing structural interference with the antigen recognizing or CDR regions. Protein M binds antibodies and prevents antigen-antibody union but does not disrupt previously formed antigen-antibody complexes. The interaction of protein M with antibodies has been validated by western blot, ELISA, x-ray crystallography, electron microscopy, and biolayer interferometry. Since protein M can be used independently of the vector, it could be incorporated into a treatment regimen that includes previously FDA approved gene therapies. This is an advantage over capsid based immune evasion, since each individual capsid must go through its own clinical trial for each disease target. Based on the properties of protein M, this protein can be not only applied for any viral vector mediated gene delivery, but also for transient protein therapy such as CRISPR/cas9 in case to avoid humoral immune response mediated clearance. Also, protein M has potential to treat autoimmune disorders resulted from autoantibodies.

In the present study, it was demonstrated that at least 2 or more molecules of protein M are required to block one molecule of immunoglobulin, which may need high amount of protein M to interact with all immunoglobulin in blood for NAb evasion after systemic administration. Although high dose of recombinant proteins such as human serum albumin and immunoglobulin has been used in clinical trials, the concern from potential complications of high dose of protein M needs to be addressed in big animals before clinical trials. It may not be a major issue if protein M is locally used for AAV administration.

The in vitro study showed that the complex of protein M/immunoglobulin was stable for a relatively long time, while the in vivo experiment manifested a gradual loss of protein M function when AAV vectors were administered much later after protein M application. It is important to figure out the dynamics and kinetics of protein M/immunoglobulin complexes in blood; this information will provide valuable information about the window for AAV injection after administration of protein M.

The other concern is immunogenicity of protein M for repeat administration. Since protein M is a foreign protein, it will elicit a humoral immune response to produce antibodies. Protein M executes its protection function by binding all immunoglobulins and the amount of specific Ig to protein M only accounts for a very small portion of all Igs, therefore, when a high dose of protein M is used for the purpose of blocking AAV NAbs, the amount of protein M specific Igs should only neutralize a very small amount of protein M and then could not influence the function of administered protein M to protect AAV from AAV NAbs. Protein M could bind to B cell surface and has the potential to stimulate B cell proliferation. Previous studies have demonstrated that intact protein M is able to induce B cell proliferation, however, truncated protein M loses this function. Long-term follow up should be performed in vivo after protein M administration.

In conclusion, the present results demonstrate that protein M prevents immunoglobulins from neutralizing AAV when protein M is present at a molecular ratio equal to or greater than 2 molecules to 1 IgG molecule. Protection of the AAV vector is dependent on protein M interacting with immunoglobulin prior to immunoglobulin neutralization of AAV. Protein M can protect AAV from neutralization spanning a 1,000-fold range of NAb titer in vivo. This study has provided the important insight to use protein M for protection of AAV vector virions from NAbs activity in future AAV clinical trials in patients with NAbs or for re-administration.

### Example 8: Engineered Protein M Variants with Enhanced Properties

The above examples describe the use of Mycoplasma protein M as an antibody blocking protein in order to escape neutralizing antibodies against gene therapy vectors. While naturally occurring protein M homologs from different Mycoplasma species bind most mammalian antibody classes with nanomolar affinity (Grover et al., Science 343:656 (2014)) many features of the naturally occurring protein are unsuitable for use as a therapeutic. Native protein M is not soluble, but is membrane bound by an N-terminal transmembrane domain that anchors it in the bacterial plasma membrane. Additionally, the native protein has a disordered C-terminus that may behave unpredictably as a drug-like molecule. Investigations were made using a truncated version of the native protein M (protein M TD from Grover et al., Science 343:656 (2014)) lacking the N-terminal transmembrane domain and the disordered C-terminal segment as a therapeutic antibody-blocking molecule, but a key discovery was made that this protein lacks structural stability when incubated at body temperature (37° C.).

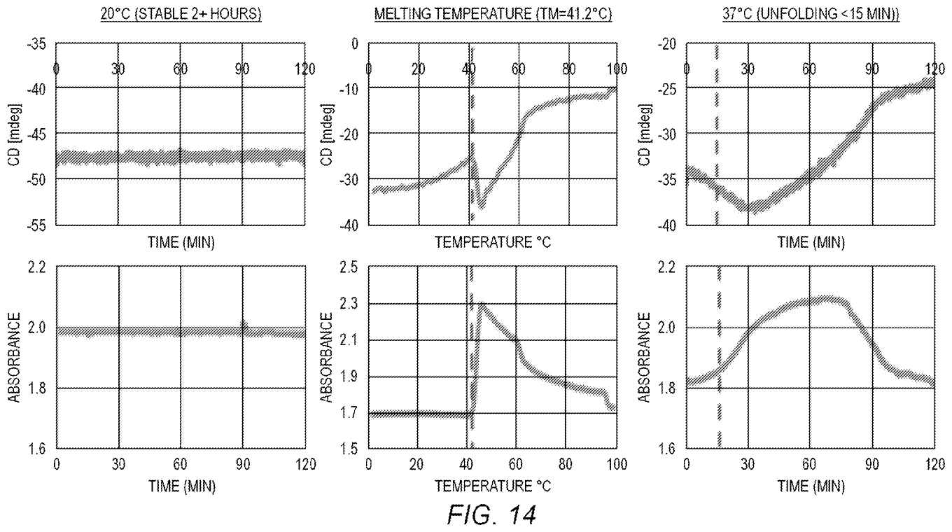

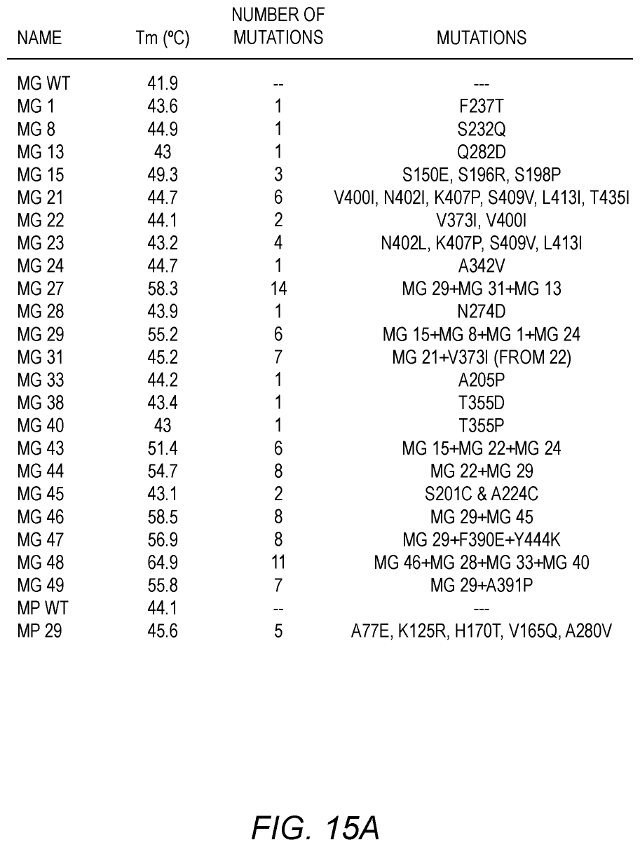

Exposing the truncated protein M (SEQ ID NO:3) to 37° C. in a simple in vitro buffer suspension caused visible precipitation and aggregation to occur within a short period of time (15 min to 1 h). Upon analysis using circular dichroism, it was discovered that heating the protein to 37° C. was associated with a distinct protein unfolding event within 1 h and instantaneous melting of the protein occurred at 41.2° C., while no unfolding event occurred at 20° C. incubation lasting over 2 h (FIG. 14). Unfolding of the protein also correlated with a measured decrease of the soluble fraction in solution assessed by western blot. To overcome therapeutic limitations of using an unstable protein, a rational design approach was used to generate mutant analogs of truncated protein M, including mutant homologs from M. genitalium (MG281) and M. pneumoniae (MPN400). This approach used crystal structures and amino acid sequences of protein M (PDB ID: 4NZR and 4NZT) as input into the protein modeling and engineering software called Rosetta. Free energy calculations and 3D modeling were used to predict which amino acid sequence changes would result in stabilization of the tertiary structure, outputting a list of over 850+ amino acid substitutions. Through DNA synthesis of individual mutants and combined multiple mutants, a rationally designed library of variants were used to produce mutant proteins. Table 4 lists 885 computationally identified point mutations that scored better than the wild-type protein for thermostability. Table 5 lists 165 computationally identified point mutations that scored substantially better (greater than −3.0 delta score) than the wild-type protein for thermostability.

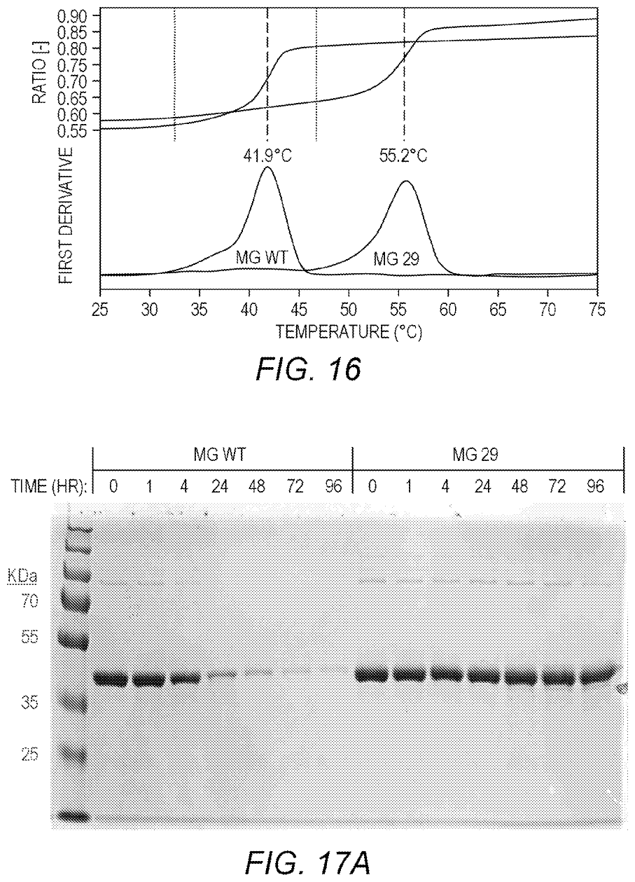

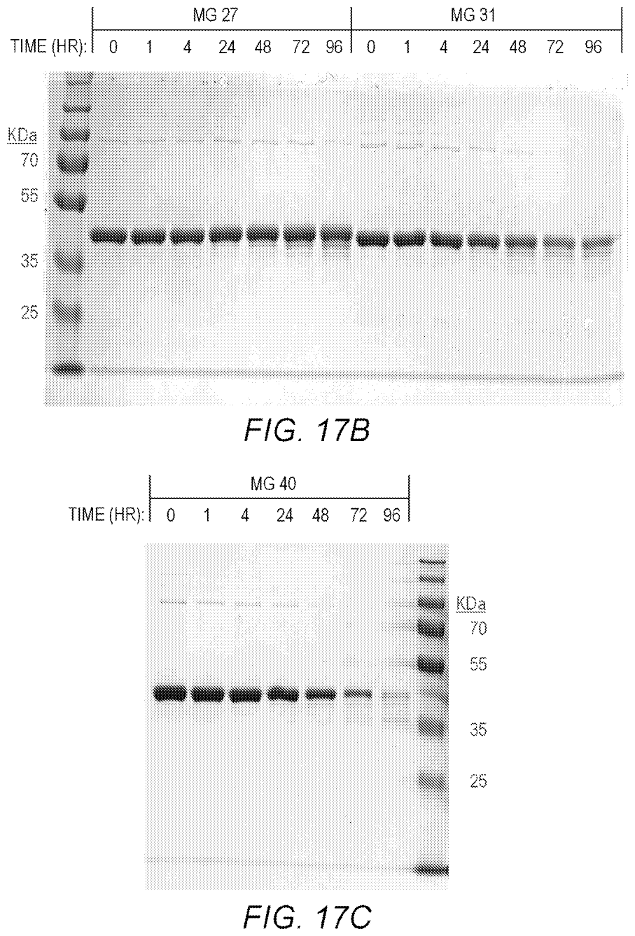

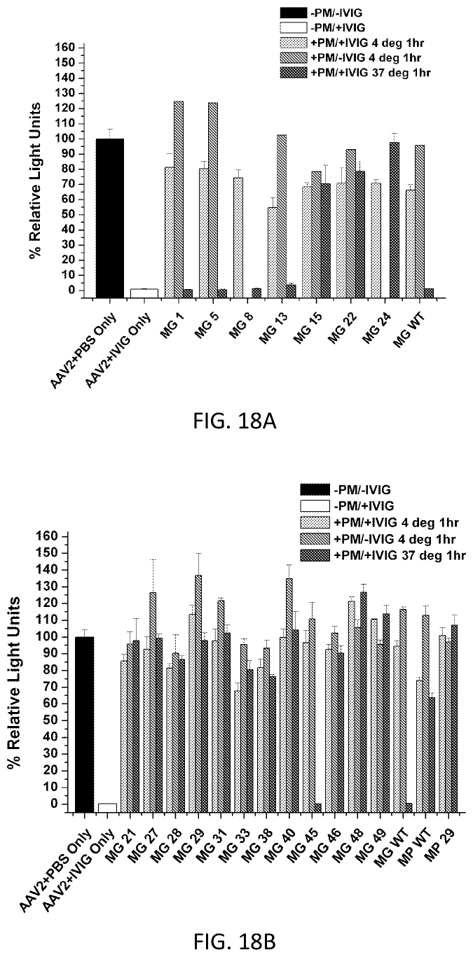

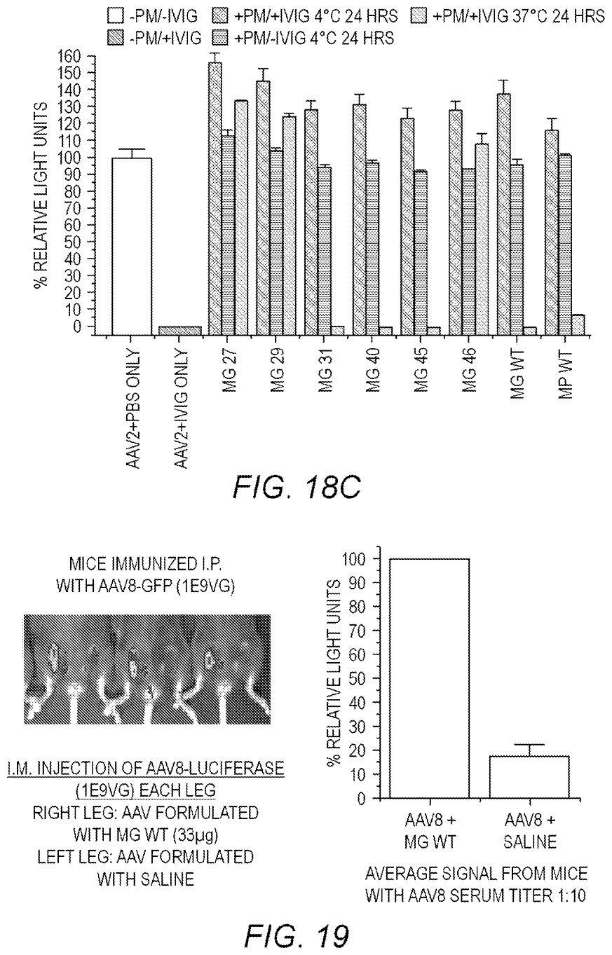

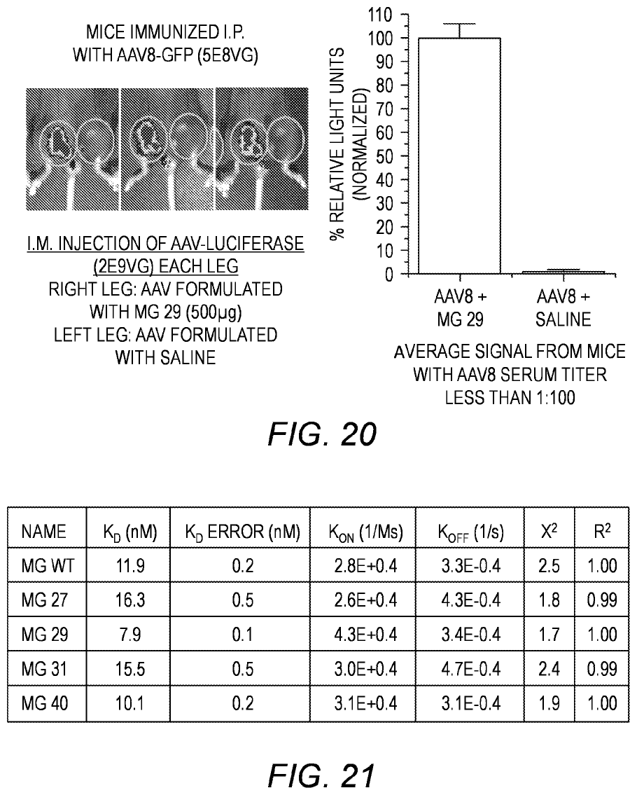

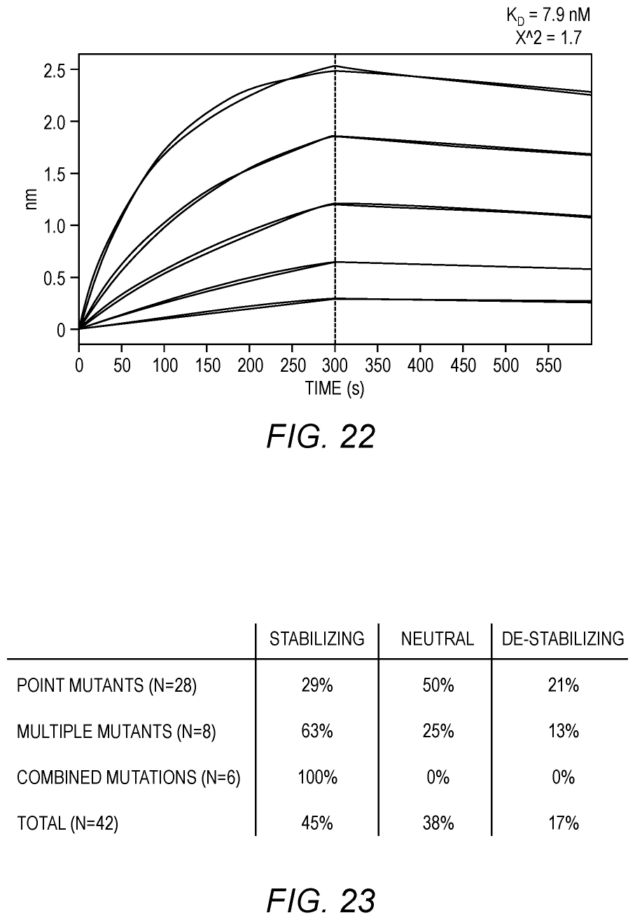

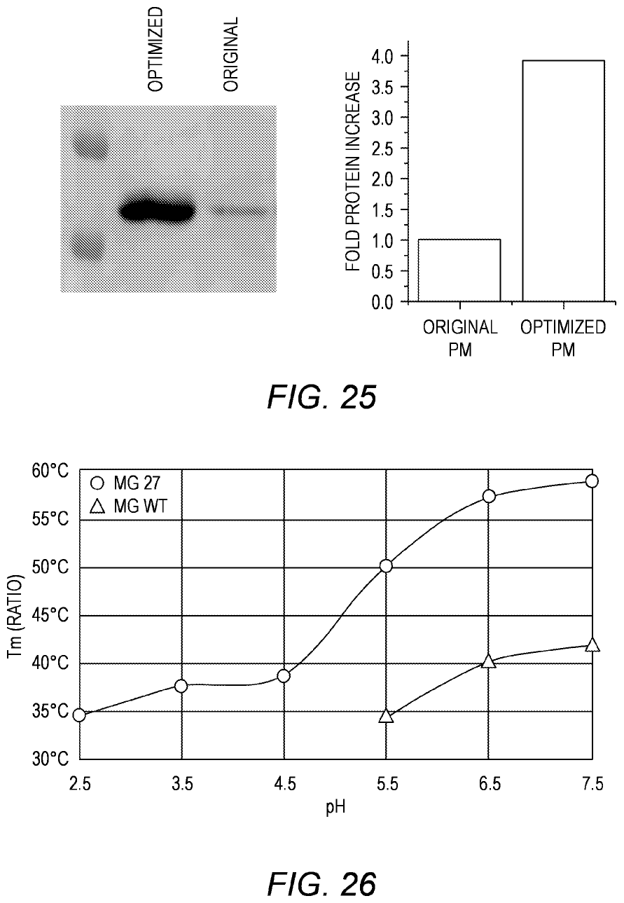

Different protein M mutants were validated using differential scanning fluoroscopy to calculate protein melting temperature (FIGS. 15, 16). Mutants were then subjected to temperature challenge at 37° C. for varying periods of time to measure solubility (FIGS. 17A-17C) and the ability to prevent AAV neutralization by pooled human intravenous IgG (IVIG) in an in vitro neutralization assay (18A-18C). In addition to testing analogs of protein M derived from M. genitalium, analogs derived from M pneumonia were also produced. Some mutants were then tested by Bio-Layer Interferometry (BLI) to determine affinity of the mutant protein to mouse IgG, and demonstrate the ability of mutants to alter affinity of the mutants for immunoglobulin substrates (FIGS. 21, 22). Mutants also showed increased stability as evidenced by stability over a broader pH range than wild-type protein M (FIG. 26). MG 29, one of the lead analogs with increased stability at 37° C. and maintenance of nano-molar affinity for IgG, was used to test blockade of AAV neutralizing antibodies in vivo after direct immunization of the mouse against the AAV capsid. Results from intramuscular injection in one leg with AAV formulated with phosphate buffered saline, and the other leg with AAV formulated with MG 29 demonstrated that MG 29 blocks AAV neutralizing antibodies in AAV immunized mice to enable effective gene delivery and redosing of AAV (FIGS. 19, 20).

A second round of rational engineering strategies using Rosetta software included alteration of mutant protein affinity using an averaged model of many human immunoglobulin structures to predict modifications of the binding site of mutant protein M analogs to enhance or diminish affinity and binding. Furthermore, a third round of rational design strategies incorporated using both Rosetta modeling and the diversity of naturally occurring protein M sequences from different Mycoplasma species to create antigenically distinct analogs that do not exist in nature. These analogs can be chimeric, mosaic, or de-novo rationally altered amino acid mutants of the whole protein or individual epitopes. The same or different analogs can then be used in conjunction with AAV for multiple rounds of redosing, even in the presence of inhibitory antibodies generated against protein M analogs. This is because protein M analogs directly compete for antibody binding and blocking of antigen recognition, even recognition of itself by inhibitory antibodies. Saturating doses of protein M analogs will be in excess of the small portion of immunoglobulins raised against protein M analogs after initial immune exposure. Additionally, generation of protein M analogs with increased epitope diversity can provide an additional layer of immune evasion from protein M inhibitory antibodies. It is believed that each of the listed engineering strategies can be combined or overlayed on top of each other to create protein M analogs with multiple different properties.

Finally, DNA codon optimized versions of protein M were produced that enhanced protein product yield (FIG. 25). Two codon optimized versions of the parental truncated protein M sequence have been generated: one sequence optimized for both E. coli and H. sapiens codon usage, and the other optimized for H. sapiens codon usage only. The dual E. coli and H. sapiens codon construct was used to produce protein in E. coli and demonstrates a ˜4-fold increase in protein yield compared to the parental unoptimized plasmid. The H. sapiens only optimized construct contains an IL-2 secretion peptide or albumin secretion peptide sequence to enable secretion of protein from mammalian cell lines for protein M analog harvest from the culture media. Protein M mutant analogs are cloned or synthesized into both types of optimized DNA sequences, and used for enhanced protein production. Protein M analogs that are stable at 37° C. can be grown in human cells without susceptibility to protein unfolding after secretion. Additionally, glycosylation sites of protein M analogs are substituted to eliminate glycosylation at the binding site or add glycosylation to the external surface, such as analog MG 28 (N274D). Addition of glycosylation sites to the external surface is another mechanism to enhance immune evasion of protein M analogs and reduce protein M inhibitory antibody recognition for multiple administration of protein M analogs. Finally, DNA sequences of protein M analogs will be stably integrated into mammalian cell lines in order to produce secreted protein that is stable at 37° C. and has glycosylation sites either removed or new ones added.

### Methods

AAV virus production: AAV vectors were produced using a standard approach with three-plasmid transfection in HEK293 cells. Briefly, the AAV transgene plasmid pTR-CBA-Luc was co-transfected with an AAV Rep/Cap helper plasmid (pXR2 or pXR8) and adenovirus helper plasmid pXX6-80. 72 h later, cell cultures were harvested and lysed by freeze thaw and ultra sonication. Clarified cell lysate was DNAse treated and ultra-centrifuged in a 15%/25%/40%/60% iodixanol step gradient and purified by anion exchange Q-column. Purified AAV vector was titered by qPCR with primers directed to amplify a segment of the packaged AAV transgene.

Protein Production: The plasmid pET-28b(+) which encodes the protein M analogs, truncated proteins derived from M. genitalium or M pneumonia lacking the transmembrane domain, and carries a N-terminal His-Tag and a thrombin cleavage site. Plasmids were propagated in electro-competent DH10B cells and purified using a PureLink Maxi-Prep kit from Invitrogen. The pET-28b(+) plasmid was transiently transfected into BL21/DE3 cells using an overnight starter culture. Auto-induction media (Magic media from Invitrogen) was seeded with the starter culture and grown at 18° C. for 3 days. The culture was then pelleted by centrifugation and frozen down at −80° C.

Protein Purification: The frozen bacterial cell culture pellet was thawed, lysed by sonication, DNAse treated, and clarified by centrifugation. Clarified bacterial lysate was dialyzed into nickel-binding buffer (20 mM imidazole, 50 mM sodium phosphate pH 7.4, 500 mM NaCl, 0.02% sodium azide) and passed through a nickel His-trap FF column using FPLC. Protein M bound by the nickel column was then eluted by the same buffer base but containing 500 mM imidazole. Protein M was then run through an S-100 size exclusion column, dialyzed into phosphate buffered saline with 2% glycerol, and quantified using spectrophotometry. Protein M identity was confirmed using SDS-PAGE protein gel electrophoresis for protein separation followed by a coomassie blue protein stain to correctly identify the 45 kDa-48 kDa protein M band.

For some protein production, either of two strategies were implemented to purify the protein M variants. The first protocol (Ni-Purity) was for small-scale production for NanoDSF. Lysis was performed by mixing cell pellets with a buffer consisting of 2.5 mg/mL Lysozyme, 25% B-PER, 75% Phosphate Buffered Saline (PBS) pH 7.4, and protease inhibitors (PMSF, Bestatin, and Pepstatin). After 30 min of mixing at room temperature, the crude lysates were centrifuged at 15,000 ref for 20 min and the supernatant was incubated with Ni resin for 1 h at 4° C. The resin was then washed (PBS+20 mM imidazole) and the protein was eluted with (PBS+500 mM imidazole). The eluted protein was exchanged into PBS pH 7.4 with Zeba desalting columns before conducting the stability assay. The second protocol (SEC-Purity) was for large-scale production and removed additional contaminants/aggregates from the protein samples. The cells were lysed via sonication, purified with nickel resin (same buffers as the first protocol), and finished with size-exclusion chromatography into PBS+2% glycerol before freezing the samples.

Western Blot: After protein separation on an 8% SDS-PAGE gel, the proteins were transferred onto a PVDF membrane. Immunoblotting was performed in 5% non-fat milk using anti-His primary antibody 1:1000 dilution (10 μg/ml). The secondary goat anti-human IgG antibody conjugated to horseradish peroxidase (1:10,000 dilution).

Determination of Protein Melting Temperature and Unfolding: Melting temperatures (Tm) were determined with nano differential scanning fluorimetry (NanoDSF), and protein unfolding was determined at different temperatures using a circular dichroism assay. Inflection points of the first derivative indicate Tm or unfolding. Proteins were purified according to protocol 1 (Ni-Purity).

Solubility Assay: A thermostability time-course was performed by incubating seven aliquots of each SEC-purified protein (0.4 mg/mL) at 37° C. for varying amounts of time. Precipitated protein was pelleted by centrifuging the samples at 15,000×G for 10 minutes before loading a gel for SDS-PAGE. Proteins were purified according to protocol 2 (SEC-Purity).

Affinity Assessment by Bio-Layer Interferometry: Bio-Layer Interferometry was performed at 37° C. to assess the affinity of the Protein M constructs. Proteins were purified according to protocol 2 (SEC-Purity). A two-fold dilution series of each M construct ranging from 1000 nM to 15.6 nM was performed with ForteBio Kinetics buffer (PBS+detergent). Binding was assessed to Anti-Mouse IgG Fc Capture biosensors. Each protocol included a baseline, association, and dissociation step for 10, 5, and 5 minutes, respectively. Data was subtracted from a reference sensor run in parallel with an association step in buffer. Affinity data were calculated based on a 1:1 curve fit within the ForteBio Data Analysis v9.0 software. Reported steady-state binding constants were calculated based on the maximum response at each concentration (n=7). To minimize X2, the kinetic data only included data for the 250-15.6 nM dilution range (n=5).

Structural Modeling and Rational Protein Engineering: Optimization of analogs of protein M (PM) thermal stability was achieved using an in silico rational protein engineering approach based on existing crystal structures (4NZT and 4NZR) of PM bound to immunoglobulins. The Rosetta software package was employed to identify mutations that stabilize the PM peptide. The first protocol performed site saturation mutagenesis in silico, allowing the amino acid neighbors to repack around the mutated residue. A second protocol generated combinatorial mutations in regions that are under-packed within the crystal structure. The mutants were ranked according to the difference in score between the wild type amino acid and the substitutions. Additional visual inspection was performed to identify preferred mutants. The mutations were cloned into the pET-28b+ vector by Twist Biosciences and synthesized. Additional design strategies for alteration of binding site affinity and generation of PM analogs with distinct antigenicity were employed.

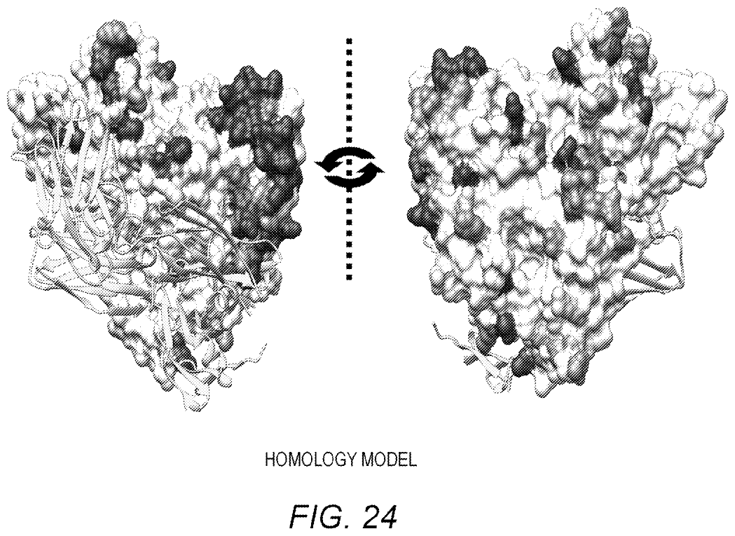

A homology model of MP WT was constructed with the Swiss-Model webserver (FIG. 24). Conservation scores between the MG and MP WT isoforms were calculated according to the BLOSUM90 matrix. The image was rendered in PyMol.

Cell Culture: HEK-293 cells or Huh7 cells were used for all in vitro AAV neutralization experiments. Cells were grown on 15 cm tissue culture plates and maintained at 37° C. in 5% CO₂ with Dulbecco's modified Eagle's medium supplemented with 10% bovine calf serum and penicillin-streptomycin.

Human IVIG for in vitro neutralization experiments: A stock of 10% Human IVIG (Gamunex) was purchased from Grifols Therapeutics Inc. (Research Triangle Park, NC, USA). Individual aliquots were diluted to 1 mg/mL in phosphate buffered saline and stored at −80° C. for future use. Serum was collected and pooled from 12 different mice (50% male, 50% female) after IP administration of 3×10¹⁰ viral genomes of AAV8-FVIII followed by a boost administration of the same vector 2 weeks later, and a second boost 6 weeks after the first administration. Mouse serum were aliquoted and stored at −80° C. for future use.

In vitro AAV Neutralization Assay: Nab analyses were performed as described previously with slight modification (Wang et al., Gene Ther. 22:984 (2015)). Cells were pelleted by centrifugation and resuspended in serum free X-Vivo 10 medium, then plated in either a 48-well or 96-well plate format. Human IVIG or serum was serially diluted either two-fold or ten-fold. AAV-Luc was incubated with either human IVIG or mouse serum for 1 h at 4° C. before adding AAV and incubating an additional 1 h at 4° C. AAV+serum incubations were then mixed with cells suspended in serum free media at the time of plating. In the neutralization experiments using protein M, 3 different incubations scenarios were tested with each step for 1 h at 4° C.: neutralizing serum incubated first with protein M followed by AAV, AAV incubated first with neutralizing serum followed by protein M, and AAV incubated first with protein M followed by neutralizing serum. Cells were seeded in a 48-well plate in 200 μl media, or in a 96-well plate in 100 μl media. After transduction, cells were cultured for 24-48 h at 37° C. to allow for AAV-Luciferase transgene expression. To measure Luc activity, cells were lysed with passive lysis buffer (Promega, Madison, WI, USA) and luciferase signal was measured with a Wallac1420 Victor 2 automated plate reader. Nab titers were defined as the highest dilution for which luciferase activity was 50% lower than serum-free controls.

In Vivo AAV Redosing in AAV Immunized Mice: C57BL/6 mice were immunized by I.P. injection of either 8E5vg or 1E9vg AAV8-GFP. Approximately 1-month later serum was collected from the mice for titering by AAV8 in vitro neutralization assay. A day later I.M. injection was performed in each leg containing equal doses of either 1E9vg or 2E9vg AAV8-Luciferase, where one leg was administered AAV formulated with phosphate buffered saline, and the other leg was administered AAV formulated with a protein M analog in a simple admixture just prior to injection. A total volume of 200 μl was injected per leg. Luminescent imaging was performed 2-weeks after AAV-Luc administration after I.P. administration of D-Luciferin substrate.

### Example 9: Kinetics of Modified Protein M and Fusion Proteins with Protein M

Biolayer interferometry was performed on a serial dilution of protein M concentrations. FIG. 22 shows exemplary data for MG29. Data from MG29 and other Protein M constructs are summarized in Tables 8, 9 and FIG. 21.

TABLE 8

| | KD | | | Full | Full | Max Conc. | # Points |
|---|---|---|---|---|---|---|---|
| Sample | (nM) | kon(1/Ms) | kdis(1/s) | X\hat{ }2 | R\hat{ }2 | (nM) | in Fit |
| WT* | 0.80 | 8.38E+04 | 6.73E−05 | 1.55 | 1.00 | 50 | 6 |
| 21* | 0.74 | 5.23E+04 | 3.85E−05 | 0.48 | 1.00 | 50 | 6 |
| 29* | 0.92 | 8.00E+04 | 7.35E−05 | 0.80 | 1.00 | 50 | 6 |
| 27* | 1.05 | 5.65E+04 | 5.95E−05 | 0.61 | 1.00 | 50 | 6 |
| 66* | 0.69 | 8.01E+04 | 5.55E−05 | 0.84 | 1.00 | 50 | 6 |

TABLE 9

| Sample | KD | | | Full | Full | Max Conc. | Num |
|---|---|---|---|---|---|---|---|
| ID | (nM) | kon(1/Ms) | kdis(1/s) | X\hat{ }2 | R\hat{ }2 | (nM) | Dilutions |
| MG64 | <0.001 | 2.21E+04 | <1.0E−07 | 0.09 | 1.00 | 500 | 5 |
| MG29 | 7.9 | 4.3E+04 | 3.4E−04 | 1.7 | 1.00 | 250 | 5 |

Anti-human IgG Fc capture (AHC) biosensors and anti-mouse IgG Fc capture biosensors (AMC) were purchased from ForteBio. The experiments were performed at 37° C. with 10 min association and dissociation. The results were fit with a 1:1 binding curve. An asterisk* denotes amino acid Y338. Analogs without an * are N338.

### Example 10: In Vivo Antibody Neutralization

Serum was collected and pooled from mice serially immunized with AAV8. In vitro neutralization assay determined that the anti-AAV8 neutralizing titer of the pooled serum to be roughly 1:10,000. Different quantities of the anti-AAV8 serum (determined by serum volume) were administered via IV injection to groups of AAV naive mice in a passive transfer experiment, followed 20-25 minutes later by IV injection of 2e10 viral genomes (vg) of AAV8-Luciferase reporter vector. Some groups of mice were given protein M via IV injection 5 minutes before the AAV administration. Non-invasive in vivo imaging of luciferase activity was performed 7 days later to quantify luminescence as a proxy of gene expression from the AAV8 vector, and any decrease in luminescence signal is indicative of AAV8-Luciferase neutralization by the serum. Results are shown in FIG. 29. Mice given 0.0003 ul of serum failed to neutralize the AAV8 and resulted in nearly 100% luminescence signal as compared to naive mice without serum injected with 2e10 vg AAV8-Luciferase alone. Groups of mice given increasing quantities of neutralizing serum neutralized progressively more and more AAV8-Luciferase, where 0.001 ul to 0.003 ul serum neutralized approximately half of the AAV and 0.3 ul serum or greater neutralized over 99% of the AAV. Groups of mice administered 3 ul of serum, followed by MG88 or MG118 and then 2e10 vg AAV8-Luciferase, resulted in gene expression indicative of escape from neutralization due to suppression of neutralizing antibodies by the protein M fusion proteins. The specific doses of MG88, MG29, MG29*, and MGWT* were 6.3 mg and the dose of MG118 was 9 mg per mouse, with an average mouse weight of 21 grams.

### Example 11: Thermostability of Disulfide Mutations of Protein M

Mutations were added to a stabilized version of protein M (Start) that included the following mutations: S150E, S196R, S198P, F237T, S232Q, A342V, N274D, A205P, and T355P (Table 10). Melting temperatures (Tm) were determined with NanoDSF. Two capillaries were run on the same sample (Tm1 & Tm2). ΔTm represents the difference between the average of Tm1 & Tm2 compared to the Start construct.

TABLE 10

| Internal | | | | | |
|---|---|---|---|---|---|
| Number | Name | Tm1 | Tm2 | ΔTm | Mutations* |
| 66 | Start* | 61.2° C. | 61.2° C. | 0 | |
| 48 | Disulf1 | 64.9° C. | 64.9° C. | 3.7 | S201C & A224C |
| 87 | Disulf2 | 64.6° C. | 64.5° C. | 3.4 | Q159C & A182C |
| 91 | Disulf3 | 60.8° C. | 60.7° C. | −0.5 | A102C & T161C |
| 92 | Disulf4 | 61.0° C. | 61.1° C. | −0.1 | I86C & F101C |
| 112 | Disulf1&2 | 68.6° C. | 68.5° C. | 7.3 | |

### Example 12: In Vitro Antibody Neutralization

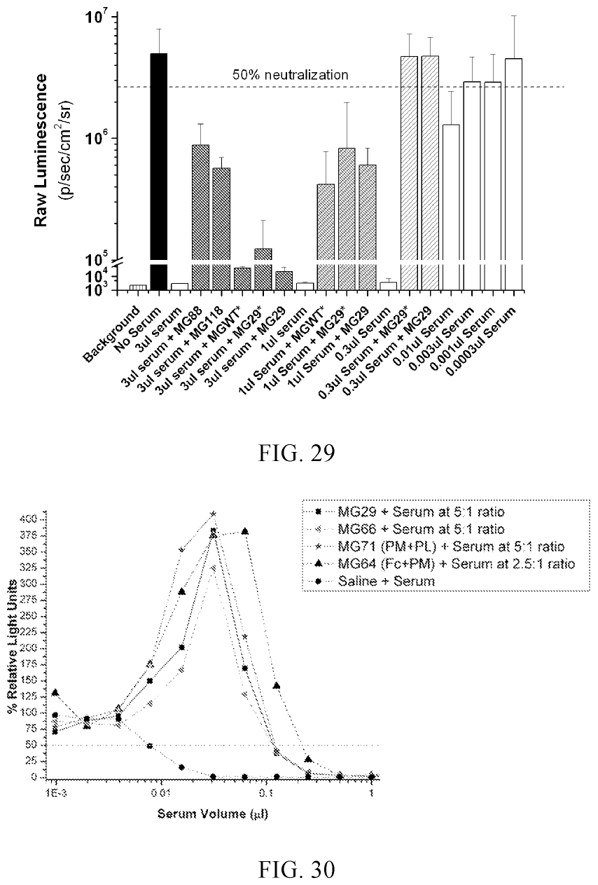

FIG. 30 shows an in vitro neutralization assay where high titer anti-AAV8 serum from mice was titrated in 2-fold dilutions, and added to the well as indicated on the X-axis. Then the different Protein M analogs (MG29, MG66, MG71, and MG64) were added to the well in the molecular ratio indicated in the figure legend and incubated for 1 hour. Saline was added to the serum only well (black circles). Then AAV8-Luciferase (2×10¹⁰ viral genomes) was added to each well and incubated 1 hr, before adding 5×10⁴ Huh7 cells in serum free media. The cells were cultured 48 hrs and then the luciferase reporter signal, which is a proxy for neutralization, was read and quantified.

This data demonstrates these analogs have neutralizing antibody blocking functionality, thereby shifting the neutralization curve, and that the MG64 Fc-Protein M fusion dimer (Fc-PM) shifts the curve greater than the Protein M monomer analogs. Based on biolayer inferometry, MG64 has sub-nanomolar affinity for immunoglobulin.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

TABLE 4
885 point mutations predicted to improve stability of MG WT. Delta
Score refers to the score of the mutant − score of the WT
residue. This list considered residues 78-468 EXCEPT for residues
within 5 A of the antibody interface within PDB ID: 4NZR.

| Delta | WT | Residue | Mutant |
|---|---|---|---|
| Score | Residue | Number | Residue |
| −1.941 | N | 78 | A |
| −1.209 | N | 78 | D |
| −2.37 | N | 78 | K |
| −2.042 | N | 78 | P |
| −0.503 | N | 78 | R |
| −2.53 | S | 81 | D |
| −1.123 | S | 81 | Q |
| −1.913 | Q | 83 | F |
| −1.083 | Q | 83 | H |
| −0.893 | Q | 83 | K |
| −2.313 | Q | 83 | W |
| −3.922 | Q | 83 | Y |
| −0.688 | S | 84 | I |
| −0.532 | S | 84 | N |
| −1.437 | S | 84 | T |
| −0.66 | E | 85 | G |
| −0.797 | E | 85 | M |
| −0.601 | S | 89 | R |
| −0.891 | G | 90 | A |
| −1.407 | G | 90 | D |
| −2.616 | G | 90 | E |
| −2.329 | G | 90 | F |
| −2.57 | G | 90 | H |
| −2.587 | G | 90 | K |
| −1.955 | G | 90 | L |
| −0.961 | G | 90 | M |
| −2.926 | G | 90 | Q |
| −1.886 | G | 90 | R |
| −1.851 | G | 90 | S |
| −0.542 | G | 90 | T |
| −0.794 | G | 90 | W |
| −3.813 | G | 90 | Y |
| −1.071 | G | 91 | D |
| −0.548 | G | 91 | S |
| −1.752 | A | 92 | D |
| −1.759 | A | 92 | E |
| −1.217 | A | 92 | F |
| −3.819 | A | 92 | G |
| −2.717 | A | 92 | H |
| −1.365 | A | 92 | K |
| −2.814 | A | 92 | L |
| −1.631 | A | 92 | M |
| −0.523 | A | 92 | N |
| −2.818 | A | 92 | Q |
| −2.113 | A | 92 | R |
| −1.364 | A | 92 | S |
| −1.66 | A | 92 | T |
| −1.995 | A | 92 | W |
| −3.614 | A | 92 | Y |
| −0.571 | N | 93 | D |
| −0.867 | N | 93 | E |
| −3.022 | F | 94 | T |
| −0.67 | F | 94 | V |
| −1.789 | E | 96 | D |
| −0.618 | K | 97 | Y |
| −0.729 | N | 100 | E |
| −0.506 | N | 100 | G |
| −0.817 | N | 100 | K |
| −0.705 | N | 100 | S |
| −0.569 | N | 100 | T |
| −2.662 | F | 101 | W |
| −0.531 | A | 108 | H |
| −1.192 | A | 108 | V |
| −1.094 | A | 108 | W |
| −0.723 | N | 111 | H |
| −1.94 | N | 111 | K |
| −1.372 | N | 111 | R |
| −0.545 | N | 111 | Y |
| −1.2 | S | 112 | A |
| −3 | S | 112 | I |
| −0.791 | S | 112 | N |
| −1.232 | S | 112 | Q |
| −2.525 | S | 112 | T |
| −1.568 | S | 112 | V |
| −2.517 | S | 112 | Y |
| −0.871 | P | 113 | E |
| −0.828 | K | 122 | Q |
| −1.059 | K | 122 | R |
| −0.876 | T | 123 | F |
| −0.857 | T | 123 | R |
| −2.035 | T | 123 | V |
| −0.542 | T | 123 | Y |
| −0.636 | I | 125 | V |
| −1.039 | S | 126 | A |
| −1.739 | S | 126 | T |
| −2.338 | S | 126 | V |
| −0.914 | S | 126 | Y |
| −1.447 | G | 127 | V |
| −0.932 | L | 128 | I |
| −0.743 | L | 128 | R |
| −0.66 | L | 128 | V |
| −0.978 | K | 130 | L |
| −0.557 | K | 130 | R |
| −1.688 | K | 130 | Y |
| −2.35 | T | 131 | L |
| −0.832 | D | 133 | W |
| −1.585 | N | 134 | I |
| −1.077 | N | 134 | K |
| −1.851 | N | 134 | L |
| −1.405 | N | 134 | M |
| −0.653 | N | 134 | R |
| −0.558 | N | 134 | W |
| −0.966 | N | 134 | Y |
| −1.303 | I | 136 | Y |
| −1.332 | T | 137 | C |
| −4.319 | T | 137 | I |
| −0.647 | T | 137 | M |
| −2.796 | T | 137 | V |
| −2.106 | S | 139 | D |
| −0.656 | S | 139 | N |
| −1.302 | S | 139 | P |
| −0.713 | K | 141 | I |
| −0.59 | K | 141 | L |
| −0.687 | K | 141 | V |
| −1.754 | K | 141 | W |
| −3.179 | A | 142 | I |
| −1.581 | A | 142 | T |
| −3.178 | A | 142 | V |
| −0.904 | D | 146 | F |
| −1.939 | D | 146 | I |
| −2.182 | D | 146 | L |
| −0.705 | D | 146 | V |
| −1.628 | D | 146 | W |
| −1.335 | D | 146 | Y |
| −5.122 | H | 147 | F |
| −2.485 | H | 147 | L |
| −2.909 | H | 147 | M |
| −0.596 | H | 147 | W |
| −3.907 | H | 147 | Y |
| −2.12 | V | 148 | F |
| −1.976 | V | 148 | I |
| −1.402 | V | 148 | L |
| −1.182 | V | 148 | Q |
| −0.697 | V | 148 | W |
| −1.809 | V | 148 | Y |
| −1.083 | A | 149 | L |
| −2.315 | A | 149 | T |
| −1.928 | A | 149 | V |
| −2.153 | S | 150 | A |
| −3.033 | S | 150 | D |
| −7.399 | S | 150 | E |
| −2.785 | S | 150 | H |
| −2.321 | S | 150 | K |
| −1.154 | S | 150 | L |
| −1.469 | S | 150 | N |
| −2.975 | S | 150 | Q |
| −1.326 | S | 150 | R |
| −0.648 | S | 150 | Y |
| −1.674 | S | 153 | R |
| −1.26 | L | 154 | I |
| −0.729 | L | 155 | R |
| −1.276 | L | 155 | Y |
| −0.943 | S | 156 | C |
| −2.615 | S | 156 | F |
| −4.064 | S | 156 | I |
| −4.341 | S | 156 | K |
| −3.463 | S | 156 | L |
| −2.776 | S | 156 | M |
| −0.936 | S | 156 | N |
| −1.309 | S | 156 | P |
| −0.846 | S | 156 | R |
| −4.236 | S | 156 | T |
| −3.759 | S | 156 | V |
| −4.005 | S | 156 | W |
| −2.623 | S | 156 | Y |
| −1.007 | F | 164 | L |
| −0.618 | F | 164 | Y |
| −0.571 | R | 167 | F |
| −2.262 | R | 167 | W |
| −1.335 | M | 170 | F |
| −2.097 | T | 175 | P |
| −0.937 | T | 176 | N |
| −1.299 | N | 184 | E |
| −1.904 | N | 184 | F |
| −2.794 | N | 184 | I |
| −1.554 | N | 184 | T |
| −3.268 | N | 184 | V |
| −1.558 | N | 184 | Y |
| −0.501 | R | 185 | F |
| −1.262 | R | 185 | I |
| −2.285 | R | 185 | L |
| −2.347 | R | 185 | M |
| −0.999 | D | 189 | Q |
| −0.831 | R | 192 | D |
| −2.382 | R | 192 | G |
| −1.519 | R | 192 | T |
| −1.168 | R | 192 | V |
| −0.527 | Q | 193 | K |
| −1.143 | Q | 193 | R |
| −0.963 | S | 196 | E |
| −1.34 | S | 196 | F |
| −1.583 | S | 196 | I |
| −1.214 | S | 196 | K |
| −1.886 | S | 196 | L |
| −0.669 | S | 196 | M |
| −0.517 | S | 196 | N |
| −3.217 | S | 196 | Q |
| −3.52 | S | 196 | R |
| −0.976 | S | 196 | V |
| −3.279 | S | 196 | Y |
| −1.692 | S | 198 | A |
| −2.806 | S | 198 | F |
| −1.083 | S | 198 | H |
| −2.867 | S | 198 | I |
| −3.325 | S | 198 | K |
| −3.234 | S | 198 | L |
| −2.335 | S | 198 | M |
| −1.628 | S | 198 | N |
| −3.686 | S | 198 | P |
| −2.556 | S | 198 | Q |
| −3.722 | S | 198 | R |
| −0.646 | S | 198 | T |
| −1.334 | S | 198 | V |
| −6.127 | S | 198 | Y |
| −1.375 | S | 201 | D |
| −1.334 | S | 201 | E |
| −1.914 | S | 201 | F |
| −1.701 | S | 201 | G |
| −0.869 | S | 201 | H |
| −1.426 | S | 201 | K |
| −1.078 | S | 201 | N |
| −1.07 | S | 201 | Q |
| −2.311 | S | 201 | T |
| −1.826 | S | 201 | V |
| −2.775 | S | 201 | Y |
| −0.788 | N | 202 | A |
| −2.384 | N | 202 | F |
| −2.528 | N | 202 | I |
| −0.825 | N | 202 | S |
| −2.151 | N | 202 | T |
| −1.783 | N | 202 | V |
| −2.941 | N | 202 | W |
| −2.296 | N | 202 | Y |
| −2.388 | K | 204 | P |
| −1.227 | K | 204 | Q |
| −0.519 | A | 205 | C |
| −2.228 | A | 205 | I |
| −1.657 | A | 205 | L |
| −3.12 | A | 205 | P |
| −1.408 | A | 205 | Q |
| −0.669 | A | 205 | T |
| −0.817 | A | 205 | V |
| −1.201 | T | 206 | D |
| −0.502 | T | 206 | E |
| −0.83 | T | 206 | F |
| −1.418 | T | 206 | I |
| −0.654 | T | 206 | L |
| −0.73 | T | 206 | Q |
| −1.067 | T | 206 | V |
| −1.448 | T | 206 | Y |
| −0.588 | T | 207 | R |
| −0.717 | S | 209 | E |
| −1.577 | S | 209 | F |
| −1.686 | S | 209 | I |
| −0.958 | S | 209 | K |
| −1.783 | S | 209 | L |
| −1.567 | S | 209 | M |
| −0.791 | S | 209 | Q |
| −1.454 | S | 209 | R |
| −1.638 | S | 209 | T |
| −1.984 | S | 209 | V |
| −1.384 | S | 209 | W |
| −2.308 | S | 209 | Y |
| −1.891 | S | 211 | D |
| −3.197 | S | 211 | K |
| −0.9 | S | 211 | N |
| −0.542 | S | 211 | P |
| −2.202 | S | 211 | Q |
| −1.875 | T | 215 | F |
| −1.234 | T | 215 | H |
| −1.064 | T | 215 | Q |
| −1.375 | T | 215 | W |
| −3.349 | T | 215 | Y |
| −0.856 | K | 218 | I |
| −0.888 | K | 218 | R |
| −1.308 | K | 218 | V |
| −0.656 | T | 220 | F |
| −1.52 | T | 220 | I |
| −1.372 | T | 220 | K |
| −0.646 | T | 220 | L |
| −0.945 | T | 220 | R |
| −1.702 | T | 220 | V |
| −0.666 | T | 220 | W |
| −0.891 | T | 220 | Y |
| −1.113 | F | 222 | R |
| −2.105 | A | 224 | E |
| −1.543 | A | 224 | Q |
| −0.698 | K | 225 | E |
| −3.025 | K | 225 | P |
| −1.562 | G | 226 | D |
| −0.743 | S | 227 | T |
| −2.441 | D | 231 | C |
| −3.049 | D | 231 | I |
| −2.346 | D | 231 | K |
| −0.937 | D | 231 | L |
| −0.738 | D | 231 | R |
| −3.258 | D | 231 | T |
| −0.971 | S | 232 | A |
| −1.24 | S | 232 | C |
| −5.712 | S | 232 | D |
| −6.694 | S | 232 | E |
| −3.533 | S | 232 | F |
| −3.132 | S | 232 | H |
| −6.649 | S | 232 | I |
| −4.075 | S | 232 | K |
| −6.718 | S | 232 | L |
| −4.104 | S | 232 | M |
| −3.012 | S | 232 | N |
| −6.497 | S | 232 | Q |
| −4.977 | S | 232 | R |
| −3.937 | S | 232 | V |
| −1.104 | S | 232 | W |
| −1.517 | S | 232 | Y |
| −1.935 | P | 234 | A |
| −3.303 | P | 234 | D |
| −1.38 | P | 234 | E |
| −1.214 | P | 234 | F |
| −3.816 | P | 234 | K |
| −2.003 | P | 234 | L |
| −4.159 | P | 234 | Q |
| −4.154 | P | 234 | R |
| −2.314 | P | 234 | S |
| −2.296 | P | 234 | T |
| −0.924 | P | 234 | W |
| −1.709 | P | 234 | Y |
| −3.955 | N | 235 | W |
| −0.751 | H | 236 | A |
| −4.755 | H | 236 | F |
| −3.111 | H | 236 | I |
| −1.635 | H | 236 | K |
| −3.932 | H | 236 | M |
| −2.882 | H | 236 | T |
| −5.392 | H | 236 | V |
| −2.542 | H | 236 | W |
| −3.223 | H | 236 | Y |
| −1.625 | F | 237 | C |
| −3.832 | F | 237 | D |
| −3.074 | F | 237 | E |
| −3.752 | F | 237 | I |
| −4.054 | F | 237 | K |
| −2.101 | F | 237 | L |
| −1.774 | F | 237 | N |
| −1.465 | F | 237 | P |
| −1.24 | F | 237 | Q |
| −4.499 | F | 237 | R |
| −3.617 | F | 237 | T |
| −3.417 | F | 237 | V |
| −2.078 | E | 239 | D |
| −4.044 | E | 239 | F |
| −1.146 | E | 239 | I |
| −0.577 | E | 239 | K |
| −0.717 | E | 239 | L |
| −1.1 | E | 239 | R |
| −0.829 | E | 239 | S |
| −0.933 | E | 239 | T |
| −0.887 | E | 239 | V |
| −1.193 | E | 239 | W |
| −3.362 | E | 239 | Y |
| −2.128 | V | 241 | I |
| −0.684 | V | 241 | M |
| −2.872 | T | 243 | I |
| −3.525 | T | 243 | V |
| −1.174 | L | 244 | M |
| −2.811 | A | 245 | D |
| −1.161 | A | 245 | E |
| −1.134 | A | 245 | F |
| −3.259 | A | 245 | I |
| −3.917 | A | 245 | L |
| −2.947 | A | 245 | M |
| −5.98 | A | 245 | Q |
| −2.926 | A | 245 | R |
| −0.579 | A | 245 | S |
| −4.056 | A | 245 | V |
| −0.829 | I | 246 | Y |
| −1.007 | D | 247 | E |
| −1.005 | D | 247 | F |
| −1.068 | D | 247 | G |
| −2.402 | D | 247 | H |
| −1.724 | D | 247 | I |
| −0.822 | D | 247 | L |
| −0.793 | D | 247 | M |
| −1.467 | D | 247 | N |
| −1.068 | D | 247 | V |
| −1.243 | D | 247 | W |
| −1.512 | D | 247 | Y |
| −0.559 | K | 249 | N |
| −1.533 | K | 249 | R |
| −2.761 | D | 250 | E |
| −0.872 | D | 250 | F |
| −1.268 | D | 250 | H |
| −1.039 | D | 250 | I |
| −0.698 | D | 250 | M |
| −1.318 | D | 250 | N |
| −3.546 | D | 250 | Q |
| −0.744 | D | 250 | R |
| −3.286 | D | 250 | W |
| −1.838 | D | 250 | Y |
| −1.447 | S | 252 | G |
| −2.581 | A | 253 | K |
| −0.599 | A | 253 | N |
| −1.189 | A | 253 | Q |
| −1.419 | A | 253 | R |
| −0.52 | L | 254 | H |
| −4.297 | K | 255 | C |
| −2.226 | K | 255 | F |
| −0.65 | K | 255 | G |
| −4.582 | K | 255 | I |
| −6.318 | K | 255 | L |
| −4.961 | K | 255 | M |
| −1.004 | K | 255 | N |
| −1.368 | K | 255 | Q |
| −1.305 | K | 255 | R |
| −1.12 | K | 255 | T |
| −2.993 | K | 255 | V |
| −0.79 | K | 255 | W |
| −2.48 | K | 255 | Y |
| −1.663 | T | 256 | A |
| −4.605 | T | 256 | D |
| −3.077 | T | 256 | E |
| −2.088 | T | 256 | F |
| −1.105 | T | 256 | H |
| −1.273 | T | 256 | I |
| −2.344 | T | 256 | K |
| −1.261 | T | 256 | L |
| −2.018 | T | 256 | N |
| −1.721 | T | 256 | Q |
| −1.135 | T | 256 | R |
| −1.085 | T | 256 | S |
| −3.685 | T | 256 | W |
| −3.064 | T | 256 | Y |
| −1.052 | T | 257 | E |
| −0.905 | T | 257 | F |
| −1.179 | T | 257 | H |
| −1.169 | T | 257 | L |
| −2.225 | T | 257 | M |
| −1.898 | T | 257 | V |
| −0.719 | T | 257 | W |
| −2.493 | T | 257 | Y |
| −1.846 | I | 258 | F |
| −0.602 | D | 259 | I |
| −2.52 | D | 259 | L |
| −2.002 | D | 259 | M |
| −2.419 | D | 259 | N |
| −4.395 | D | 259 | R |
| −0.504 | D | 259 | T |
| −1.555 | D | 259 | Y |
| −3.113 | T | 264 | D |
| −0.619 | T | 264 | G |
| −0.606 | T | 264 | H |
| −1.036 | T | 264 | I |
| −2.271 | T | 264 | K |
| −2.231 | T | 264 | N |
| −1.027 | T | 264 | Q |
| −1.297 | T | 264 | R |
| −1.789 | T | 264 | V |
| −1.162 | R | 269 | F |
| −2.384 | R | 269 | W |
| −0.916 | R | 269 | Y |
| −1.668 | G | 270 | K |
| −1.018 | G | 270 | N |
| −7.501 | S | 272 | G |
| −6.59 | N | 274 | D |
| −1.29 | N | 274 | H |
| −1.438 | N | 274 | K |
| −1.345 | N | 274 | Q |
| −1.128 | N | 274 | R |
| −1.046 | N | 274 | S |
| −6.425 | G | 275 | A |
| −6.719 | G | 275 | C |
| −3.004 | G | 275 | E |
| −3.439 | G | 275 | F |
| −2.989 | G | 275 | H |
| −5.834 | G | 275 | L |
| −5.771 | G | 275 | M |
| −4.083 | G | 275 | N |
| −2.832 | G | 275 | Q |
| −5.141 | G | 275 | S |
| −6.48 | S | 276 | D |
| −4.126 | S | 276 | E |
| −4.296 | S | 276 | F |
| −3.057 | S | 276 | G |
| −1.099 | S | 276 | H |
| −1.014 | S | 276 | L |
| −1.983 | S | 276 | M |
| −4.191 | S | 276 | Q |
| −1.244 | S | 276 | R |
| −4.129 | S | 276 | T |
| −2.589 | S | 276 | W |
| −3.747 | S | 276 | Y |
| −1.295 | N | 279 | D |
| −1.316 | N | 279 | E |
| −1.933 | N | 279 | F |
| −0.519 | N | 279 | M |
| −2.816 | N | 279 | R |
| −1.667 | N | 279 | S |
| −2.151 | N | 279 | W |
| −5.197 | N | 279 | Y |
| −5.082 | Q | 282 | D |
| −1.912 | Q | 282 | E |
| −3.221 | Q | 282 | G |
| −2.608 | Q | 282 | H |
| −3.413 | Q | 282 | N |
| −1.175 | P | 284 | A |
| −0.563 | P | 284 | C |
| −1.726 | P | 284 | D |
| −0.948 | P | 284 | R |
| −0.824 | S | 286 | D |
| −0.7 | S | 286 | T |
| −0.836 | V | 287 | I |
| −1.126 | V | 287 | L |
| −0.679 | K | 288 | R |
| −1.935 | S | 291 | T |
| −4.25 | T | 297 | D |
| −5.165 | T | 297 | G |
| −2.506 | T | 297 | H |
| −0.602 | T | 297 | K |
| −0.653 | T | 297 | L |
| −3.168 | T | 297 | N |
| −3.251 | T | 297 | Q |
| −0.569 | T | 297 | R |
| −0.607 | T | 297 | Y |
| −0.622 | V | 299 | I |
| −3.283 | N | 300 | A |
| −0.67 | N | 300 | D |
| −2.389 | N | 300 | E |
| −4.801 | N | 300 | F |
| −0.955 | N | 300 | G |
| −2.175 | N | 300 | H |
| −2.688 | N | 300 | K |
| −1.342 | N | 300 | L |
| −1.566 | N | 300 | M |
| −4.67 | N | 300 | Q |
| −4.369 | N | 300 | R |
| −2.253 | N | 300 | T |
| −3.152 | N | 300 | W |
| −4.116 | N | 300 | Y |
| −0.711 | A | 302 | D |
| −1.057 | A | 302 | K |
| −2.367 | A | 302 | N |
| −3.445 | A | 302 | P |
| −1.437 | A | 302 | Q |
| −1.562 | A | 302 | R |
| −1.241 | A | 302 | S |
| −1.665 | A | 302 | T |
| −0.918 | A | 302 | V |
| −1.631 | K | 303 | M |
| −1.841 | Q | 304 | P |
| −1.3 | I | 305 | L |
| −0.931 | A | 307 | G |
| −1.57 | N | 308 | I |
| −1.084 | N | 308 | R |
| −1.003 | N | 308 | Y |
| −0.919 | V | 309 | I |
| −1.968 | V | 309 | L |
| −3.222 | V | 310 | D |
| −4.277 | V | 310 | E |
| −1.284 | V | 310 | G |
| −2.278 | V | 310 | I |
| −1.206 | V | 310 | K |
| −2.64 | V | 310 | L |
| −1.981 | V | 310 | M |
| −1.643 | V | 310 | Q |
| −0.603 | V | 310 | R |
| −1.476 | V | 310 | Y |
| −0.969 | E | 311 | D |
| −0.741 | E | 313 | T |
| −0.584 | T | 317 | L |
| −1.56 | T | 317 | Q |
| −0.974 | S | 318 | T |
| −1.689 | K | 319 | G |
| −1.245 | K | 319 | P |
| −0.645 | K | 319 | Q |
| −0.662 | K | 319 | S |
| −1.445 | A | 320 | N |
| −2.732 | A | 320 | S |
| −3.457 | A | 320 | T |
| −1.693 | S | 322 | D |
| −1.149 | S | 322 | E |
| −2.31 | S | 322 | F |
| −0.547 | S | 322 | L |
| −1.208 | S | 322 | Q |
| −1.229 | S | 322 | R |
| −1.363 | S | 322 | W |
| −2.665 | S | 322 | Y |
| −1.806 | N | 326 | C |
| −1.348 | N | 326 | E |
| −6.807 | N | 326 | I |
| −3.008 | N | 326 | L |
| −4.117 | N | 326 | M |
| −0.912 | N | 326 | Q |
| −3.518 | N | 326 | T |
| −6.143 | N | 326 | V |
| −1.18 | N | 326 | W |
| −1.944 | P | 327 | I |
| −0.929 | P | 327 | L |
| −1.154 | V | 329 | I |
| −1.555 | G | 331 | C |
| −7.293 | G | 331 | D |
| −1.759 | G | 331 | E |
| −2.826 | G | 331 | H |
| −0.631 | G | 331 | I |
| −1.148 | G | 331 | K |
| −2.247 | G | 331 | M |
| −5.615 | G | 331 | N |
| −3.757 | G | 331 | Q |
| −1.709 | G | 331 | R |
| −5.193 | G | 331 | S |
| −6.058 | G | 331 | T |
| −2.025 | G | 331 | V |
| −1.709 | S | 332 | D |
| −4.232 | S | 332 | G |
| −1.172 | K | 333 | D |
| −0.711 | K | 333 | F |
| −1.439 | K | 333 | H |
| −1.286 | K | 333 | I |
| −0.779 | K | 333 | M |
| −0.688 | K | 333 | N |
| −0.984 | K | 333 | Q |
| −0.517 | K | 333 | R |
| −0.911 | K | 333 | V |
| −2.311 | N | 335 | F |
| −0.994 | N | 335 | H |
| −1.861 | N | 335 | I |
| −0.569 | N | 335 | K |
| −1.288 | N | 335 | Q |
| −1.41 | N | 335 | R |
| −1.375 | N | 335 | V |
| −2.167 | N | 335 | W |
| −4.264 | N | 335 | Y |
| −1.842 | I | 337 | L |
| −2.65 | A | 342 | F |
| −2.784 | A | 342 | I |
| −3.644 | A | 342 | L |
| −4.974 | A | 342 | V |
| −2.055 | S | 343 | F |
| −1.081 | S | 343 | H |
| −2.498 | S | 343 | I |
| −0.647 | S | 343 | L |
| −1.148 | S | 343 | M |
| −2.983 | S | 343 | T |
| −5.103 | S | 343 | V |
| −3.4 | T | 347 | L |
| −0.929 | T | 347 | Y |
| −2.024 | H | 348 | F |
| −1.537 | H | 348 | I |
| −3.436 | H | 348 | L |
| −0.527 | H | 348 | T |
| −0.875 | H | 348 | W |
| −2.087 | H | 348 | Y |
| −0.64 | L | 351 | W |
| −2.674 | L | 351 | Y |
| −0.9 | V | 354 | I |
| −0.894 | V | 354 | W |
| −2.123 | T | 355 | A |
| −6.547 | T | 355 | D |
| −2.908 | T | 355 | E |
| −0.505 | T | 355 | F |
| −6.168 | T | 355 | G |
| −1.467 | T | 355 | H |
| −1.937 | T | 355 | I |
| −2.431 | T | 355 | K |
| −1.713 | T | 355 | L |
| −1.509 | T | 355 | M |
| −2.926 | T | 355 | N |
| −4.599 | T | 355 | P |
| −2.548 | T | 355 | Q |
| −1.312 | T | 355 | R |
| −1.839 | T | 355 | S |
| −1.912 | T | 355 | V |
| −0.851 | T | 355 | W |
| −3.208 | T | 355 | Y |
| −1.345 | Q | 357 | A |
| −2.15 | Q | 357 | D |
| −1.018 | Q | 357 | E |
| −2.172 | Q | 357 | F |
| −0.616 | Q | 357 | K |
| −0.946 | Q | 357 | L |
| −0.974 | Q | 357 | M |
| −2.632 | Q | 357 | N |
| −3.698 | Q | 357 | P |
| −1.047 | Q | 357 | R |
| −3.433 | Q | 357 | S |
| −1.903 | Q | 357 | T |
| −2.693 | Q | 357 | W |
| −3.419 | Q | 357 | Y |
| −1.398 | N | 358 | A |
| −0.821 | N | 358 | T |
| −0.616 | N | 358 | V |
| −1.062 | N | 358 | Y |
| −1.438 | S | 359 | D |
| −1.059 | S | 359 | E |
| −1.378 | S | 359 | K |
| −2.104 | S | 359 | R |
| −1.28 | D | 360 | N |
| −2.902 | N | 361 | D |
| −3.808 | N | 361 | G |
| −0.729 | N | 361 | Q |
| −3.46 | N | 361 | S |
| −0.856 | S | 362 | T |
| −0.513 | A | 363 | F |
| −0.78 | A | 363 | N |
| −0.774 | A | 363 | R |
| −0.558 | A | 363 | T |
| −0.668 | A | 363 | Y |
| −1.701 | N | 367 | D |
| −2.021 | N | 367 | E |
| −2.692 | N | 367 | R |
| −1.089 | N | 367 | T |
| −1.429 | L | 369 | F |
| −1.334 | L | 369 | Y |
| −0.878 | K | 370 | Q |
| −1.132 | Q | 371 | E |
| −4.072 | Q | 371 | G |
| −1.78 | Q | 371 | K |
| −0.776 | Q | 371 | N |
| −1.402 | Q | 371 | S |
| −1.278 | Q | 371 | T |
| −1.584 | A | 372 | F |
| −1.167 | A | 372 | M |
| −0.734 | A | 372 | Q |
| −1.863 | A | 372 | Y |
| −1.946 | V | 373 | F |
| −1.549 | V | 373 | I |
| −0.698 | V | 373 | L |
| −2.254 | V | 373 | W |
| −2.226 | V | 373 | Y |
| −2.604 | G | 374 | A |
| −0.584 | G | 374 | C |
| −1.9 | G | 374 | D |
| −2.244 | G | 374 | E |
| −1.248 | G | 374 | F |
| −2.467 | G | 374 | H |
| −2.244 | G | 374 | I |
| −2.238 | G | 374 | K |
| −2.56 | G | 374 | L |
| −2.589 | G | 374 | M |
| −1.682 | G | 374 | N |
| −2.086 | G | 374 | Q |
| −1.912 | G | 374 | R |
| −2.461 | G | 374 | S |
| −1.343 | G | 374 | T |
| −1.743 | G | 374 | V |
| −1.968 | G | 374 | W |
| −3.292 | G | 374 | Y |
| −2.883 | D | 375 | E |
| −2.01 | D | 375 | F |
| −2.704 | D | 375 | Y |
| −1.033 | N | 378 | D |
| −2.421 | N | 378 | F |
| −0.527 | N | 378 | H |
| −4.306 | N | 378 | I |
| −2.095 | N | 378 | L |
| −2.91 | N | 378 | V |
| −2.298 | N | 378 | W |
| −5.452 | N | 378 | Y |
| −1.64 | Q | 385 | I |
| −1.14 | Q | 385 | L |
| −6.284 | Q | 385 | Y |
| −1.657 | L | 399 | Y |
| −2.025 | V | 400 | I |
| −1.497 | V | 400 | L |
| −0.776 | V | 400 | M |
| −4.382 | K | 401 | G |
| −0.99 | N | 402 | D |
| −0.666 | N | 402 | H |
| −3.648 | N | 402 | I |
| −0.629 | N | 402 | K |
| −3.676 | N | 402 | L |
| −1.291 | N | 402 | M |
| −4.123 | N | 402 | Q |
| −1.9 | N | 402 | R |
| −0.632 | N | 402 | T |
| −2.535 | N | 402 | V |
| −2.104 | N | 402 | Y |
| −1.381 | T | 405 | D |
| −1.16 | T | 405 | E |
| −1.456 | T | 405 | G |
| −0.661 | T | 405 | N |
| −1.568 | T | 405 | P |
| −0.659 | T | 405 | S |
| −1.621 | N | 406 | D |
| −1.036 | K | 407 | Q |
| −0.624 | D | 408 | E |
| −1.367 | S | 409 | A |
| −1.017 | S | 409 | D |
| −1.012 | S | 409 | E |
| −2.884 | S | 409 | F |
| −2.887 | S | 409 | G |
| −3.223 | S | 409 | H |
| −2.537 | S | 409 | I |
| −2.811 | S | 409 | K |
| −1.925 | S | 409 | L |
| −2.672 | S | 409 | N |
| −0.58 | S | 409 | Q |
| −2.919 | S | 409 | R |
| −2.456 | S | 409 | V |
| −1.9 | S | 409 | W |
| −3.769 | S | 409 | Y |
| −0.703 | D | 411 | I |
| −0.601 | D | 411 | N |
| −1.019 | D | 411 | W |
| −2.19 | D | 411 | Y |
| −3.008 | L | 413 | I |
| −0.851 | L | 413 | R |
| −0.557 | L | 413 | T |
| −2.019 | V | 414 | F |
| −1.445 | V | 414 | W |
| −2.099 | V | 414 | Y |
| −1.502 | S | 417 | A |
| −1.726 | S | 417 | L |
| −1.482 | S | 417 | M |
| −1.798 | S | 417 | T |
| −2.722 | S | 417 | V |
| −2.372 | S | 417 | W |
| −1.977 | S | 417 | Y |
| −1.184 | L | 418 | F |
| −0.84 | L | 418 | Y |
| −0.759 | K | 419 | E |
| −1.798 | K | 419 | I |
| −2.514 | K | 419 | L |
| −1.942 | K | 419 | M |
| −1.308 | K | 419 | Q |
| −3.11 | H | 424 | F |
| −1.633 | H | 424 | L |
| −1.004 | H | 424 | M |
| −3.125 | H | 424 | Y |
| −0.972 | A | 428 | C |
| −1.093 | A | 428 | I |
| −1.48 | A | 428 | L |
| −2.906 | A | 428 | T |
| −2.715 | A | 428 | V |
| −2.242 | N | 434 | A |
| −1.994 | N | 434 | F |
| −1.556 | N | 434 | G |
| −1.092 | N | 434 | I |
| −0.835 | N | 434 | L |
| −1.969 | N | 434 | Q |
| −2.092 | N | 434 | R |
| −1.518 | N | 434 | T |
| −0.503 | N | 434 | V |
| −0.931 | T | 435 | I |
| −1.41 | T | 435 | V |
| −1.764 | Y | 443 | E |
| −2.209 | Y | 450 | W |
| −0.751 | S | 459 | K |
| −1.234 | S | 459 | L |
| −1.791 | S | 459 | M |
| −2.107 | S | 459 | P |
| −0.934 | E | 460 | D |
| −3.904 | E | 460 | G |
| −1.489 | E | 460 | K |
| −1.046 | E | 460 | Q |
| −1.919 | E | 460 | R |
| −0.581 | N | 463 | A |
| −2.529 | N | 463 | D |
| −3.986 | N | 463 | E |
| −4.058 | N | 463 | K |
| −0.527 | N | 463 | L |
| −2.682 | N | 463 | Q |
| −2.561 | N | 463 | R |
| −0.51 | N | 463 | T |
| −1.797 | N | 463 | V |
| −3.545 | E | 464 | A |
| −3.37 | E | 464 | D |
| −2.285 | E | 464 | H |
| −2.454 | E | 464 | I |
| −5.122 | E | 464 | K |
| −2.639 | E | 464 | L |
| −3.909 | E | 464 | M |
| −2.144 | E | 464 | N |
| −2.078 | E | 464 | Q |
| −3.742 | E | 464 | R |
| −2.292 | E | 464 | S |
| −2.385 | E | 464 | T |
| −1.377 | E | 464 | W |
| −0.612 | E | 464 | Y |
| −2.49 | I | 465 | L |
| −1.611 | I | 465 | Y |
| −1.788 | R | 468 | G |
| −0.909 | R | 468 | K |
| −1.169 | R | 468 | L |
| −4.089 | R | 468 | Q |
| −0.664 | R | 468 | S |
| −1.506 | R | 468 | T |
| −1.095 | R | 468 | W |

TABLE 5
165 point mutations predicted to improve stability of MG WT. Delta
Score refers to the score of the mutant − score of the WT
residue. This list considered residues 78-468 EXCEPT for residues
within 5 A of the antibody interface within PDB ID: 4NZR.

| Delta | WT | Residue | Mutant |
|---|---|---|---|
| Score | Residue | Number | Residue |
| −3.9 | Q | 83 | Y |
| −3.8 | G | 90 | Y |
| −3.8 | A | 92 | G |
| −3.6 | A | 92 | Y |
| −3.0 | F | 94 | T |
| −4.3 | T | 137 | I |
| −3.2 | A | 142 | I |
| −3.2 | A | 142 | V |
| −5.1 | H | 147 | F |
| −3.9 | H | 147 | Y |
| −3.0 | S | 150 | D |
| −7.4 | S | 150 | E |
| −4.1 | S | 156 | I |
| −4.3 | S | 156 | K |
| −3.5 | S | 156 | L |
| −4.2 | S | 156 | T |
| −3.8 | S | 156 | V |
| −4.0 | S | 156 | W |
| −3.3 | N | 184 | V |
| −3.2 | S | 196 | Q |
| −3.5 | S | 196 | R |
| −3.3 | S | 196 | Y |
| −3.3 | S | 198 | K |
| −3.2 | S | 198 | L |
| −3.7 | S | 198 | P |
| −3.7 | S | 198 | R |
| −6.1 | S | 198 | Y |
| −3.1 | A | 205 | P |
| −3.2 | S | 211 | K |
| −3.3 | T | 215 | Y |
| −3.0 | K | 225 | P |
| −3.0 | D | 231 | I |
| −3.3 | D | 231 | T |
| −5.7 | S | 232 | D |
| −6.7 | S | 232 | E |
| −3.5 | S | 232 | F |
| −3.1 | S | 232 | H |
| −6.6 | S | 232 | I |
| −4.1 | S | 232 | K |
| −6.7 | S | 232 | L |
| −4.1 | S | 232 | M |
| −3.0 | S | 232 | N |
| −6.5 | S | 232 | Q |
| −5.0 | S | 232 | R |
| −3.9 | S | 232 | V |
| −3.3 | P | 234 | D |
| −3.8 | P | 234 | K |
| −4.2 | P | 234 | Q |
| −4.2 | P | 234 | R |
| −4.0 | N | 235 | W |
| −4.8 | H | 236 | F |
| −3.1 | H | 236 | I |
| −3.9 | H | 236 | M |
| −5.4 | H | 236 | V |
| −3.2 | H | 236 | Y |
| −3.8 | F | 237 | D |
| −3.1 | F | 237 | E |
| −3.8 | F | 237 | I |
| −4.1 | F | 237 | K |
| −4.5 | F | 237 | R |
| −3.6 | F | 237 | T |
| −3.4 | F | 237 | V |
| −4.0 | E | 239 | F |
| −3.4 | E | 239 | Y |
| −3.5 | T | 243 | V |
| −3.3 | A | 245 | I |
| −3.9 | A | 245 | L |
| −6.0 | A | 245 | Q |
| −4.1 | A | 245 | V |
| −3.5 | D | 250 | Q |
| −3.3 | D | 250 | W |
| −4.3 | K | 255 | C |
| −4.6 | K | 255 | I |
| −6.3 | K | 255 | L |
| −5.0 | K | 255 | M |
| −4.6 | T | 256 | D |
| −3.1 | T | 256 | E |
| −3.7 | T | 256 | W |
| −3.1 | T | 256 | Y |
| −4.4 | D | 259 | R |
| −3.1 | T | 264 | D |
| −7.5 | S | 272 | G |
| −6.6 | N | 274 | D |
| −6.4 | G | 275 | A |
| −6.7 | G | 275 | C |
| −3.0 | G | 275 | E |
| −3.4 | G | 275 | F |
| −5.8 | G | 275 | L |
| −5.8 | G | 275 | M |
| −4.1 | G | 275 | N |
| −5.1 | G | 275 | S |
| −6.5 | S | 276 | D |
| −4.1 | S | 276 | E |
| −4.3 | S | 276 | F |
| −3.1 | S | 276 | G |
| −4.2 | S | 276 | Q |
| −4.1 | S | 276 | T |
| −3.7 | S | 276 | Y |
| −5.2 | N | 279 | Y |
| −5.1 | Q | 282 | D |
| −3.2 | Q | 282 | G |
| −3.4 | Q | 282 | N |
| −4.3 | T | 297 | D |
| −5.2 | T | 297 | G |
| −3.2 | T | 297 | N |
| −3.3 | T | 297 | Q |
| −3.3 | N | 300 | A |
| −4.8 | N | 300 | F |
| −4.7 | N | 300 | Q |
| −4.4 | N | 300 | R |
| −3.2 | N | 300 | W |
| −4.1 | N | 300 | Y |
| −3.4 | A | 302 | P |
| −3.2 | V | 310 | D |
| −4.3 | V | 310 | E |
| −3.5 | A | 320 | T |
| −6.8 | N | 326 | I |
| −3.0 | N | 326 | L |
| −4.1 | N | 326 | M |
| −3.5 | N | 326 | T |
| −6.1 | N | 326 | V |
| −7.3 | G | 331 | D |
| −5.6 | G | 331 | N |
| −3.8 | G | 331 | Q |
| −5.2 | G | 331 | S |
| −6.1 | G | 331 | T |
| −4.2 | S | 332 | G |
| −4.3 | N | 335 | Y |
| −3.6 | A | 342 | L |
| −5.0 | A | 342 | V |
| −5.1 | S | 343 | V |
| −3.4 | T | 347 | L |
| −3.4 | H | 348 | L |
| −6.5 | T | 355 | D |
| −6.2 | T | 355 | G |
| −4.6 | T | 355 | P |
| −3.2 | T | 355 | Y |
| −3.7 | Q | 357 | P |
| −3.4 | Q | 357 | S |
| −3.4 | Q | 357 | Y |
| −3.8 | N | 361 | G |
| −3.5 | N | 361 | S |
| −4.1 | Q | 371 | G |
| −3.3 | G | 374 | Y |
| −4.3 | N | 378 | I |
| −5.5 | N | 378 | Y |
| −6.3 | Q | 385 | Y |
| −4.4 | K | 401 | G |
| −3.6 | N | 402 | I |
| −3.7 | N | 402 | L |
| −4.1 | N | 402 | Q |
| −3.2 | S | 409 | H |
| −3.8 | S | 409 | Y |
| −3.0 | L | 413 | I |
| −3.1 | H | 424 | F |
| −3.1 | H | 424 | Y |
| −3.9 | E | 460 | G |
| −4.0 | N | 463 | E |
| −4.1 | N | 463 | K |
| −3.5 | E | 464 | A |
| −3.4 | E | 464 | D |
| −5.1 | E | 464 | K |
| −3.9 | E | 464 | M |
| −3.7 | E | 464 | R |
| −4.1 | R | 468 | Q |

TABLE 6
128 point mutations that are predicted to improve affinity of MG
WT to antibodies. A Delta Score refers to the score of the mutant
− score of the WT residue. This list considered 70 residues
that were within 5 Å of the antibody interface within PDB ID: 4NZR.

| Delta | WT | Residue | Mutant |
|---|---|---|---|
| Score | Residue | Number | Residue |
| −1.6 | R | 95 | E |
| −0.6 | A | 102 | I |
| −0.7 | A | 102 | L |
| −1.3 | N | 103 | A |
| −0.9 | N | 103 | C |
| −1.5 | N | 103 | D |
| −2.5 | N | 103 | E |
| −0.8 | N | 103 | F |
| −0.7 | N | 103 | H |
| −3.5 | N | 103 | I |
| −2.6 | N | 103 | K |
| −0.8 | N | 103 | L |
| −1.5 | N | 103 | M |
| −1.8 | N | 103 | Q |
| −2.5 | N | 103 | R |
| −1.0 | N | 103 | S |
| −2.3 | N | 103 | T |
| −2.1 | N | 103 | V |
| −1.3 | N | 103 | Y |
| −0.8 | S | 106 | A |
| −0.9 | E | 107 | K |
| −0.7 | E | 107 | L |
| −0.7 | K | 114 | F |
| −0.7 | K | 114 | L |
| −1.1 | K | 114 | M |
| −2.1 | K | 114 | T |
| −1.6 | L | 116 | D |
| −1.4 | L | 116 | E |
| −2.4 | L | 116 | K |
| −3.9 | L | 116 | R |
| −1.0 | L | 116 | W |
| −1.3 | S | 160 | A |
| −1.5 | S | 160 | P |
| −0.9 | T | 161 | I |
| −0.9 | T | 161 | L |
| −2.3 | T | 161 | P |
| −0.6 | E | 162 | L |
| −0.5 | Y | 163 | F |
| −1.0 | M | 181 | F |
| −0.8 | M | 181 | I |
| −1.8 | M | 181 | W |
| −0.6 | G | 186 | A |
| −0.5 | G | 186 | C |
| −0.5 | G | 186 | D |
| −1.6 | G | 186 | F |
| −2.3 | G | 186 | H |
| −0.8 | G | 186 | K |
| −0.5 | G | 186 | L |
| −0.6 | G | 186 | Q |
| −2.4 | G | 186 | R |
| −3.0 | G | 186 | S |
| −3.0 | G | 186 | T |
| −1.8 | N | 321 | H |
| −0.8 | N | 321 | K |
| −1.9 | R | 381 | F |
| −1.6 | R | 381 | W |
| −2.0 | R | 381 | Y |
| −0.7 | R | 384 | L |
| −0.5 | Y | 389 | A |
| −2.3 | Y | 389 | D |
| −2.5 | Y | 389 | E |
| −1.7 | Y | 389 | K |
| −1.1 | Y | 389 | L |
| −2.4 | Y | 389 | P |
| −1.2 | Y | 389 | Q |
| −0.8 | Y | 389 | S |
| −0.8 | F | 390 | Y |
| −2.9 | A | 391 | I |
| −0.8 | A | 391 | T |
| −2.8 | A | 391 | V |
| −1.7 | D | 396 | A |
| −0.9 | K | 397 | I |
| −0.8 | K | 397 | V |
| −1.6 | E | 426 | I |
| −1.0 | E | 426 | L |
| −0.9 | Y | 429 | E |
| −0.9 | Y | 436 | L |
| −1.5 | R | 438 | F |
| −1.6 | R | 438 | I |
| −0.6 | R | 438 | Q |
| −0.7 | R | 438 | V |
| −3.0 | V | 439 | F |
| −1.2 | V | 439 | L |
| −1.4 | V | 439 | Y |
| −1.1 | E | 441 | Q |
| −1.0 | N | 442 | D |
| −3.5 | N | 442 | E |
| −4.0 | N | 442 | F |
| −0.6 | N | 442 | H |
| −1.7 | N | 442 | I |
| −1.2 | N | 442 | L |
| −1.2 | N | 442 | M |
| −1.9 | N | 442 | V |
| −1.6 | N | 442 | W |
| −4.4 | N | 442 | Y |
| −1.0 | A | 447 | L |
| −0.5 | A | 447 | N |
| −0.8 | S | 448 | Y |
| −0.7 | I | 449 | K |
| −2.5 | I | 449 | R |
| −0.7 | N | 452 | A |
| −3.5 | N | 452 | F |
| −1.1 | N | 452 | K |
| −0.6 | N | 452 | Q |
| −1.1 | E | 453 | K |
| −1.3 | E | 453 | L |
| −2.0 | E | 453 | Q |
| −1.7 | E | 453 | R |
| −0.6 | A | 455 | M |
| −2.0 | S | 456 | A |
| −3.7 | S | 456 | F |
| −4.7 | S | 456 | K |
| −0.6 | S | 456 | L |
| −1.7 | S | 456 | M |
| −1.0 | S | 456 | N |
| −3.2 | S | 456 | R |
| −1.3 | S | 456 | Y |
| −0.8 | Q | 462 | A |
| −0.6 | Q | 462 | F |
| −1.4 | Q | 462 | K |
| −1.8 | Q | 462 | L |
| −2.7 | Q | 462 | R |
| −1.1 | Q | 462 | Y |
| −0.5 | L | 466 | D |
| −1.2 | L | 466 | E |
| −1.5 | L | 466 | K |
| −1.3 | L | 466 | R |
| −0.7 | L | 466 | Y |

TABLE 7
17 point mutations that are predicted to improve affinity of MG WT
to antibodies. A delta score refers to the score of the mutant −
score of the wt residue. This list considered 70 residues that
were within 5 Å of the antibody interface within PDB ID: 4NZR.

| Delta | WT | Residue | Mutant |
|---|---|---|---|
| Score | Residue | Number | Residue |
| −0.7 | N | 103 | H |
| −2.3 | G | 186 | H |
| −1.8 | N | 321 | H |
| −0.6 | N | 442 | H |
| −1.5 | N | 103 | D |
| −1.6 | L | 116 | D |
| −0.5 | G | 186 | D |
| −2.3 | Y | 389 | D |
| −1.0 | N | 442 | D |
| −0.5 | L | 466 | D |
| −1.6 | R | 95 | E |
| −2.5 | N | 103 | E |
| −1.4 | L | 116 | E |
| −2.5 | Y | 389 | E |
| −0.9 | Y | 429 | E |
| −3.5 | N | 442 | E |
| −1.2 | L | 466 | E |

SEQUENCES

```
SEQ ID NO: 1: Wild-type M. genitalium protein M
Met Gln Phe Lys Lys His Lys Asn Ser Val Lys Phe Lys Arg Lys Leu
1 5 10 15
Phe Trp Thr Ile Gly Val Leu Gly Ala Gly Ala Leu Thr Thr Phe Ser
                        20 25 30
Ala Val Met Ile Thr Asn Leu Val Asn Gln Ser Gly Tyr Ala Leu Val
                35 40 45
Ala Ser Gly Arg Ser Gly Asn Leu Gly Phe Lys Leu Phe Ser Thr Gln
        50 55 60
Ser Pro Ser Ala Glu Val Lys Leu Lys Ser Leu Ser Leu Asn Asp Gly
65 70 75 80
Ser Tyr Gln Ser Glu Ile Asp Leu Ser Gly Gly Ala Asn Phe Arg Glu
                                85 90 95
Lys Phe Arg Asn Phe Ala Asn Glu Leu Ser Glu Ala Ile Thr Asn Ser
                        100 105 110
Pro Lys Gly Leu Asp Arg Pro Val Pro Lys Thr Glu Ile Ser Gly Leu
                115 120 125
Ile Lys Thr Gly Asp Asn Phe Ile Thr Pro Ser Phe Lys Ala Gly Tyr
        130 135 140
Tyr Asp His Val Ala Ser Asp Gly Ser Leu Leu Ser Tyr Tyr Gln Ser
145 150 155 160
Thr Glu Tyr Phe Asn Asn Arg Val Leu Met Pro Ile Leu Gln Thr Thr
                                165 170 175
Asn Gly Thr Leu Met Ala Asn Asn Arg Gly Tyr Asp Asp Val Phe Arg
                        180 185 190
Gln Val Pro Ser Phe Ser Gly Trp Ser Asn Thr Lys Ala Thr Thr Val
                195 200 205
Ser Thr Ser Asn Asn Leu Thr Tyr Asp Lys Trp Thr Tyr Phe Ala Ala
        210 215 220
Lys Gly Ser Pro Leu Tyr Asp Ser Tyr Pro Asn His Phe Phe Glu Asp
225 230 235 240
Val Lys Thr Leu Ala Ile Asp Ala Lys Asp Ile Ser Ala Leu Lys Thr
                                245 250 255
Thr Ile Asp Ser Glu Lys Pro Thr Tyr Leu Ile Ile Arg Gly Leu Ser
                        260 265 270
Gly Asn Gly Ser Gln Leu Asn Glu Leu Gln Leu Pro Glu Ser Val Lys
                275 280 285
Lys Val Ser Leu Tyr Gly Asp Tyr Thr Gly Val Asn Val Ala Lys Gln
        290 295 300
Ile Phe Ala Asn Val Val Glu Leu Glu Phe Tyr Ser Thr Ser Lys Ala
305 310 315 320
Asn Ser Phe Gly Phe Asn Pro Leu Val Leu Gly Ser Lys Thr Asn Val
                                325 330 335
Ile Tyr Asp Leu Phe Ala Ser Lys Pro Phe Thr His Ile Asp Leu Thr
                        340 345 350
Gln Val Thr Leu Gln Asn Ser Asp Asn Ser Ala Ile Asp Ala Asn Lys
                355 360 365
Leu Lys Gln Ala Val Gly Asp Ile Tyr Asn Tyr Arg Arg Phe Glu Arg
        370 375 380
Gln Phe Gln Gly Tyr Phe Ala Gly Gly Tyr Ile Asp Lys Tyr Leu Val
385 390 395 400
Lys Asn Val Asn Thr Asn Lys Asp Ser Asp Asp Asp Leu Val Tyr Arg
                                405 410 415
Ser Leu Lys Glu Leu Asn Leu His Leu Glu Glu Ala Tyr Arg Glu Gly
                        420 425 430
Asp Asn Thr Tyr Tyr Arg Val Asn Glu Asn Tyr Tyr Pro Gly Ala Ser
                435 440 445
Ile Tyr Glu Asn Glu Arg Ala Ser Arg Asp Ser Glu Phe Gln Asn Glu
        450 455 460
Ile Leu Lys Arg Ala Glu Gln Asn Gly Val Thr Phe Asp Glu Asn Ile
465 470 475 480
Lys Arg Ile Thr Ala Ser Gly Lys Tyr Ser Val Gln Phe Gln Lys Leu
                                485 490 495
Glu Asn Asp Thr Asp Ser Ser Leu Glu Arg Met Thr Lys Ala Val Glu
                        500 505 510
Gly Leu Val Thr Val Ile Gly Glu Glu Lys Phe Glu Thr Val Asp Ile
                515 520 525
Thr Gly Val Ser Ser Asp Thr Asn Glu Val Lys Ser Leu Ala Lys Glu
        530 535 540
Leu Lys Thr Asn Ala Leu Gly Val Lys Leu Lys Leu
545 550 555
SEQ ID NO: 2: Soluble form of M. genitalium protein M (amino acid
residues 37-556 of SEQ ID NO: 1) with an N-terminal 6-His tag followed
by a thrombin cleavage site
His His His His His His Ser Ser Gly Leu Val Pro Arg Gly Ser His
1 5 10 15
Met Thr Asn Leu Val Asn Gln Ser Gly Tyr Ala Leu Val Ala Ser Gly
                        20 25 30
Arg Ser Gly Asn Leu Gly Phe Lys Leu Phe Ser Thr Gln Ser Pro Ser
                35 40 45
Ala Glu Val Lys Leu Lys Ser Leu Ser Leu Asn Asp Gly Ser Tyr Gln
        50 55 60
Ser Glu Ile Asp Leu Ser Gly Gly Ala Asn Phe Arg Glu Lys Phe Arg
65 70 75 80
Asn Phe Ala Asn Glu Leu Ser Glu Ala Ile Thr Asn Ser Pro Lys Gly
                                85 90 95
Leu Asp Arg Pro Val Pro Lys Thr Glu Ile Ser Gly Leu Ile Lys Thr
                        100 105 110
Gly Asp Asn Phe Ile Thr Pro Ser Phe Lys Ala Gly Tyr Tyr Asp His
                115 120 125
Val Ala Ser Asp Gly Ser Leu Leu Ser Tyr Tyr Gln Ser Thr Glu Tyr
        130 135 140
Phe Asn Asn Arg Val Leu Met Pro Ile Leu Gln Thr Thr Asn Gly Thr
145 150 155 160
Leu Met Ala Asn Asn Arg Gly Tyr Asp Asp Val Phe Arg Gln Val Pro
                                165 170 175
Ser Phe Ser Gly Trp Ser Asn Thr Lys Ala Thr Thr Val Ser Thr Ser
                        180 185 190
Asn Asn Leu Thr Tyr Asp Lys Trp Thr Tyr Phe Ala Ala Lys Gly Ser
                195 200 205
Pro Leu Tyr Asp Ser Tyr Pro Asn His Phe Phe Glu Asp Val Lys Thr
        210 215 220
Leu Ala Ile Asp Ala Lys Asp Ile Ser Ala Leu Lys Thr Thr Ile Asp
225 230 235 240
Ser Glu Lys Pro Thr Tyr Leu Ile Ile Arg Gly Leu Ser Gly Asn Gly
                                245 250 255
Ser Gln Leu Asn Glu Leu Gln Leu Pro Glu Ser Val Lys Lys Val Ser
                        260 265 270
Leu Tyr Gly Asp Tyr Thr Gly Val Asn Val Ala Lys Gln Ile Phe Ala
                275 280 285
Asn Val Val Glu Leu Glu Phe Tyr Ser Thr Ser Lys Ala Asn Ser Phe
        290 295 300
Gly Phe Asn Pro Leu Val Leu Gly Ser Lys Thr Asn Val Ile Tyr Asp
305 310 315 320
Leu Phe Ala Ser Lys Pro Phe Thr His Ile Asp Leu Thr Gln Val Thr
                                325 330 335
Leu Gln Asn Ser Asp Asn Ser Ala Ile Asp Ala Asn Lys Leu Lys Gln
                        340 345 350
Ala Val Gly Asp Ile Tyr Asn Tyr Arg Arg Phe Glu Arg Gln Phe Gln
                355 360 365
Gly Tyr Phe Ala Gly Gly Tyr Ile Asp Lys Tyr Leu Val Lys Asn Val
        370 375 380
Asn Thr Asn Lys Asp Ser Asp Asp Asp Leu Val Tyr Arg Ser Leu Lys
385 390 395 400
Glu Leu Asn Leu His Leu Glu Glu Ala Tyr Arg Glu Gly Asp Asn Thr
                                405 410 415
Tyr Tyr Arg Val Asn Glu Asn Tyr Tyr Pro Gly Ala Ser Ile Tyr Glu
                        420 425 430
Asn Glu Arg Ala Ser Arg Asp Ser Glu Phe Gln Asn GluIle Leu Lys
                435 440 445
Arg Ala Glu Gln Asn Gly Val Thr Phe Asp Glu Asn Ile Lys Arg Ile
        450 455 460
Thr Ala Ser Gly Lys Tyr Ser Val Gln Phe Gln Lys Leu Glu Asn Asp
465 470 475 480
Thr Asp Ser Ser Leu Glu Arg Met Thr Lys Ala Val Glu Gly Leu Val
                                485 490 495
Thr Val Ile Gly Glu Glu Lys Phe Glu Thr Val Asp Ile Thr Gly Val
                        500 505 510
Ser Ser Asp Thr Asn Glu Val Lys Ser Leu Ala Lys Glu Leu Lys Thr
                515 520 525
Asn Ala Leu Gly Val Lys Leu Lys Leu
        530 535
SEQ ID NO: 3: Wild-type M. genitalium protein M fragment 74-479
SLSLNDGSYQSEIDLSGGANFREKERNFANELSEAITNSPKGLDRPVPKTEISGLIKTGDNF
ITPSFKAGYYDHVASDGSLLSYYQSTEYFNNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 4: Modified M. genitalium protein M MG1
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHTFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 5: Modified M. genitalium protein M MG8
SLSLNDGSYQSEIDLSGGANFREKERNFANELSEAITNSPKGLDRPVPKTEISGLIKTGDNF
ITPSFKAGYYDHVASDGSLLSYYQSTEYFNNRVLMPILQTTNGTLMANNRGYDDVFRQVPSF
SGWSNTKATTVSTSNNLTYDKWTYFAAKGSPLYDQYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 6: Modified M. genitalium protein M MG13
SLSLNDGSYQSEIDLSGGANFREKERNFANELSEAITNSPKGLDRPVPKTEISGLIKTGDNF
ITPSFKAGYYDHVASDGSLLSYYQSTEYFNNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELDLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 7: Modified M. genitalium protein M MG15
SLSLNDGSYQSEIDLSGGANFREKERNFANELSEAITNSPKGLDRPVPKTEISGLIKTGDNF
ITPSFKAGYYDHVAEDGSLLSYYQSTEYENNRVLMPILQTTNGTLMANNRGYDDVFRQVPRF
PGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 8: Modified M. genitalium protein M MG21
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFASKPFTHIDLTQVTLQNSDNSAIDANKLKQAVGDIYNYRRFE
RQFQGYFAGGYIDKYLIKIVNTNPDVDDDIVYRSLKELNLHLEEAYREGDNIYYRVNENYYP
GASIYENERASRDSEFQNEILKRAEQNGVTFDEN
SEQ ID NO: 9: Modified M. genitalium protein M MG22
SLSLNDGSYQSEIDLSGGANFREKERNFANELSEAITNSPKGLDRPVPKTEISGLIKTGDNF
ITPSFKAGYYDHVASDGSLLSYYQSTEYFNNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAIGDIYNYRRFE
RQFQGYFAGGYIDKYLIKNVNTNKDSDDDLVYRSLKELNLHLEEAYREGDNTYYRVNENYYP
GASIYENERASRDSEFQNEILKRAEQNGVTFDEN
SEQ ID NO: 10: Modified M. genitalium protein M MG23
SLSLNDGSYQSEIDLSGGANFREKFRNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFASKPFTHIDLTQVTLQNSDNSAIDANKLKQAVGDIYNYRRFE
RQFQGYFAGGYIDKYLVKLVNTNPDVDDDIVYRSLKELNLHLEEAYREGDNTYYRVNENYYP
GASIYENERASRDSEFQNEILKRAEQNGVTFDEN
SEQ ID NO: 11: Modified M. genitalium protein M MG24
SLSLNDGSYQSEIDLSGGANFREKERNFANELSEAITNSPKGLDRPVPKTEISGLIKTGDNF
ITPSFKAGYYDHVASDGSLLSYYQSTEYFNNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFVSKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 12: Modified M. genitalium protein M MG27
SLSLNDGSYQSEIDLSGGANFREKERNFANELSEAITNSPKGLDRPVPKTEISGLIKTGDNF
ITPSFKAGYYDHVAEDGSLLSYYQSTEYENNRVLMPILQTTNGTLMANNRGYDDVFRQVPRF
PGWSNTKATTVSTSNNLTYDKWTYFAAKGSPLYDQYPNHTFEDVKTLAIDAKDISALKTTID
SEKPTYLIIRGLSGNGSQLNELDLPESVKKVSLYGDYTGVNVAKQIFANVVELEFYSTSKAN
SFGFNPLVLGSKINVINDLFVSKPFTHIDLTQVTLQNSDNSAIDANKLKQAIGDIYNYRRFE
RQFQGYFAGGYIDKYLIKIVNTNPDVDDDIVYRSLKELNLHLEEAYREGDNIYYRVNENYYP
GASIYENERASRDSEFQNEILKRAEQNGVTFDEN
SEQ ID NO: 13: Modified M. genitalium protein M MG28
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGDGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 14: Modified M. genitalium protein M MG29
SLSLNDGSYQSEIDLSGGANFREKERNFANELSEAITNSPKGLDRPVPKTEISGLIKTGDNF
ITPSFKAGYYDHVAEDGSLLSYYQSTEYENNRVLMPILQTTNGTLMANNRGYDDVFRQVPRF
PGWSNTKATTVSTSNNLTYDKWTYFAAKGSPLYDQYPNHTFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFVSKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 15: Modified M. genitalium protein M MG31
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAIGDIYNYRRFE
RQFQGYFAGGYIDKYLIKIVNTNPDVDDDIVYRSLKELNLHLEEAYREGDNIYYRVNENYYP
GASIYENERASRDSEFQNEILKRAEQNGVTFDEN
SEQ ID NO: 16: Modified M. genitalium protein M MG33
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKPTTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 17: Modified M. genitalium protein M MG38
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVDLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 18: Modified M. genitalium protein M MG40
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVPLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 19: Modified M. genitalium protein M MG43
SLSLNDGSYQSEIDLSGGANFREKERNFANELSEAITNSPKGLDRPVPKTEISGLIKTGDNF
ITPSFKAGYYDHVAEDGSLLSYYQSTEYENNRVLMPILQTTNGTLMANNRGYDDVFRQVPRF
PGWSNTKATTVSTSNNLTYDKWTYFAAKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFVSKPFTHIDLTQVTLQNSDNSAIDANKLKQAIGDIYNYRRFE
RQFQGYFAGGYIDKYLIKNVNTNKDSDDDLVYRSLKELNLHLEEAYREGDNTYYRVNENYYP
GASIYENERASRDSEFQNEILKRAEQNGVTFDEN
SEQ ID NO: 20: Modified M. genitalium protein M MG44
SLSLNDGSYQSEIDLSGGANFREKFRNFANELSEAITNSPKGLDRPVPKTEISGLIKTGDNF
ITPSFKAGYYDHVAEDGSLLSYYQSTEYFNNRVLMPILQTTNGTLMANNRGYDDVFRQVPRF
PGWSNTKATTVSTSNNLTYDKWTYFAAKGSPLYDQYPNHTFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFVSKPFTHIDLTQVTLQNSDNSAIDANKLKQAIGDIYNYRRFE
RQFQGYFAGGYIDKYLIKNVNTNKDSDDDLVYRSLKELNLHLEEAYREGDNTYYRVNENYYP
GASIYENERASRDSEFQNEILKRAEQNGVTFDEN
SEQ ID NO: 21: Modified M. genitalium protein M MG45
SLSLNDGSYQSEIDLSGGANFREKERNFANELSEAITNSPKGLDRPVPKTEISGLIKTGDNF
ITPSFKAGYYDHVASDGSLLSYYQSTEYENNRVLMPILQTTNGTLMANNRGYDDVFRQVPSF
SGWCNTKATTVSTSNNLTYDKWTYFACKGSPLYDSYPNHFFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKINVINDLFASKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 22: Modified M. genitalium protein M MG46
SLSLNDGSYQSEIDLSGGANFREKERNFANELSEAITNSPKGLDRPVPKTEISGLIKTGDNF
ITPSFKAGYYDHVAEDGSLLSYYQSTEYFNNRVLMPILQTTNGTLMANNRGYDDVFRQVPRF
PGWCNTKATTVSTSNNLTYDKWTYFACKGSPLYDQYPNHTFEDVKTLAIDAKDISALKTTID
SEKPTYLIIRGLSGNGSQLNELQLPESVKKVSLYGDYTGVNVAKQIFANVVELEFYSTSKAN
SFGFNPLVLGSKTNVINDLFVSKPFTHIDLTQVTLQNSDNSAIDANKLKQAVGDIYNYRRFE
RQFQGYFAGGYIDKYLVKNVNTNKDSDDDLVYRSLKELNLHLEEAYREGDNTYYRVNENYYP
GASIYENERASRDSEFQNEILKRAEQNGVTFDEN
SEQ ID NO: 23: Wild-type M. pneumoniae protein M
MKLNFKIKDKKTLKRLKKGGFWALGLFGAAINAFSAVLIVNEVLRLQSGETLIASGRSGNLS
FQLYSKVNQNAKSKLNSISLTDGGYRSEIDLGDGSNFREDERNFANNLSEAITDAPKDLLRP
VPKVEVSGLIKTSSTFITPNFKAGYYDQVAADGKTLKYYQSTEYFNNRVVMPILQTTNGTLT
ANNRAYDDIFVDQGVPKFPGWFHDVDKAYYAGSNGQSEYLFKEWNYYVANGSPLYNVYPNHH
FKQIKTIAFDAPRIKQGNTDGINLNLKQRNPDYVIINGLTGDGSTLKDLELPESVKKVSIYG
DYHSINVAKQIFKNVLELEFYSTNQDNNFGFNPLVLGDHTNIIYDLFASKPFNYIDLTSLEL
KDNQDNIDASKLKRAVSDIYIRRRFERQMQGYWAGGYIDRYLVKNTNEKNVNKDNDTVYAAL
KDINLHLEETYTHGGNTMYRVNENYYPGASAYEAERATRDSEFQKEIVQRAELIGVVFEYGV
KNLRPGLKYTVKFESPQEQVALKSTDKFQPVIGSVTDMSKSVTDLIGVLRDNAEILNITNVS
KDETVVAELKEKLDRENVFQEIRT
SEQ ID NO: 24: Wild-type M. pneumoniae protein M fragment
SISLTDGGYRSEIDLGDGSNFREDERNFANNLSEAITDAPKDLLRPVPKVEVSGLIKTSSTF
ITPNFKAGYYDQVAADGKTLKYYQSTEYFNNRVVMPILQTTNGTLTANNRAYDDIFVDQGVP
KFPGWFHDVDKAYYAGSNGQSEYLFKEWNYYVANGSPLYNVYPNHHFKQIKTIAFDAPRIKQ
GNTDGINLNLKQRNPDYVIINGLTGDGSTLKDLELPESVKKVSIYGDYHSINVAKQIFKNVL
ELEFYSTNQDNNFGFNPLVLGDHTNIIYDLFASKPFNYIDLTSLELKDNQDNIDASKLKRAV
SDIYIRRRFERQMQGYWAGGYIDRYLVKNTNEKNVNKDNDTVYAALKDINLHLEETYTHGGN
TMYRVNENYYPGASAYEAERATRDSEFQKEIVQRAELIGVVFE
SEQ ID NO: 25: Polynucleotide encoding M. genitalium protein M codon-
optimized for both bacterial and human expression
ATGGGCAGCAGCCATCACCATCATCATCACAGCAGCGGTCTGGTGCCGCGTGGT
AGCCACATGAGCCTGAGCCTGAACGATGGTAGCTACCAGAGCGAGATCGACCTG
AGCGGCGGTGCCAACTTCCGTGAAAAATTCCGCAACTTTGCTAACGAGCTGAGC
GAAGCCATTACCAATAGCCCAAAGGGCCTGGATCGTCCAGTGCCGAAAACCGAG
ATCAGCGGCCTGATTAAGACCGGTGACAACTTTATCACCCCGAGCTTCAAGGCGG
GCTACTATGATCACGTGGCTGAGGACGGTAGCCTGCTGAGCTACTATCAGAGCAC
CGAGTACTTTAACAACCGTGTGCTGATGCCGATTCTGCAGACCACCAACGGCACC
CTGATGGCCAACAACCGTGGTTATGACGACGTGTTCCGTCAGGTGCCGCGTTTCC
CGGGCTGGAGCAACACCAAAGCGACCACCGTGAGCACCAGCAACAACCTGACCT
ACGACAAGTGGACCTATTTCGCCGCGAAAGGTAGCCCGCTGTACGATCAGTATCC
GAACCACACCTTTGAGGACGTGAAAACCCTGGCTATCGATGCCAAGGACATTAG
CGCGCTGAAAACCACCATCGATAGCGAAAAGCCGACCTACCTGATCATTCGCGG
TCTGAGCGGCAACGGTAGCCAGCTGAACGAGCTGCAGCTGCCGGAAAGCGTGAA
GAAAGTGAGCCTGTACGGCGACTATACCGGTGTGAACGTGGCCAAACAGATTTT
TGCGAACGTGGTGGAGCTGGAATTCTATAGCACCAGCAAGGCGAACAGCTTCGG
CTTTAACCCGCTGGTGCTGGGTAGCAAAACCAACGTGATCAACGACCTGTTCGTG
AGCAAGCCGTTCACCCACATTGACCTGACCCAGGTGACCCTGCAGAACAGCGAT
AACAGCGCGATCGACGCTAACAAGCTGAAACAGGCTGTGGGCGATATCTACAAC
TATCGTCGCTTCGAGCGTCAGTTTCAGGGTTACTTCGCCGGCGGTTACATCGATA
AGTATCTGGTGAAAAACGTGAACACCAACAAAGACAGCGACGATGACCTGGTGT
ACCGCAGCCTGAAGGAACTGAACCTGCACCTGGAGGAAGCTTATCGTGAGGGCG
ACAACACCTACTATCGCGTGAACGAAAACTACTATCCGGGTGCCAGCATCTACG
AGAACGAACGTGCGAGCCGCGATAGCGAGTTTCAGAACGAAATTCTGAAGCGTG
CGGAGCAGAACGGCGTGACCTTCGACGAAAACTAATAA
SEQ ID NO: 26: Polynucleotide encoding M. genitalium protein M codon
optimized for human expression
ATGAGCCTGAGCCTGAACGATGGCAGCTACCAGAGCGAGATCGACCTGTCTGGC
GGAGCCAACTTCAGAGAGAAGTTCAGAAACTTCGCCAACGAGCTGAGCGAGGCC
ATCACAAACAGCCCCAAAGGCCTGGACAGACCCGTGCCTAAGACAGAGATCAGC
GGCCTGATCAAGACCGGCGACAACTTCATCACCCCTAGCTTCAAGGCCGGCTACT
ACGATCACGTGGCCTCTGATGGCAGCCTGCTGAGCTACTACCAGTCCACCGAGTA
CTTCAACAACCGGGTGCTGATGCCCATCCTCCAGACCACCAATGGCACCCTGATG
GCCAACAACAGAGGCTACGACGACGTGTTCAGACAGGTGCCCAGCTTTAGCGGC
TGGTCCAATACCAAGGCCACCACCGTGTCCACCAGCAACAACCTGACCTACGAC
AAGTGGACCTACTTCGCCGCCAAGGGCAGCCCTCTGTACGACAGCTACCCCAAC
CACTTCTTCGAGGACGTGAAAACCCTGGCCATCGACGCCAAGGATATCAGCGCC
CTGAAAACCACCATCGACAGCGAGAAGCCCACCTACCTGATCATCAGAGGACTG
AGCGGCAACGGCAGCCAGCTGAATGAACTCCAGCTGCCTGAGAGCGTGAAGAAG
GTGTCCCTGTACGGCGATTACACCGGCGTGAACGTGGCCAAGCAGATCTTCGCCA
ATGTGGTGGAACTGGAATTCTACAGCACCAGCAAGGCCAACAGCTTCGGCTTCA
ACCCTCTGGTGCTGGGCAGCAAGACCAACGTGATCAACGACCTGTTCGCCAGCA
AGCCCTTCACACACATCGATCTGACCCAAGTGACCCTCCAGAACAGCGACAACA
GCGCCATTGATGCCAACAAGCTGAAACAGGCCGTGGGCGACATCTACAACTACA
GAAGATTCGAGCGGCAGTTCCAGGGCTACTTCGCTGGCGGCTACATCGACAAGT
ACCTGGTCAAGAACGTGAACACCAACAAGGACAGCGACGACGACCTGGTGTACA
GAAGCCTGAAAGAGCTGAACCTGCACCTGGAAGAGGCCTACAGAGAGGGCGAC
AACACCTACTACAGAGTGAACGAGAACTACTACCCAGGCGCCAGCATCTACGAG
AACGAGAGAGCCAGCAGAGACAGCGAGTTCCAGAACGAGATCCTGAAGCGGGC
CGAGCAGAATGGCGTGACCTTCGACGAGAACTGATGA
SEQ ID NO: 27: Modified M. genitalium protein M MG47
SLSLNDGSYQSEIDLSGGANFREKFRNFANELSEAITNSPKGLDRPVPKTEISGLIKTG
DNFITPSFKAGYYDHVAEDGSLLSYYQSTEYFNNRVLMPILQTTNGTLMANNRGYD
DVFRQVPRFPGWSNTKATTVSTSNNLTYDKWTYFAAKGSPLYDQYPNHTFEDVKTL
AIDAKDISALKTTIDSEKPTYLIIRGLSGNGSQLNELQLPESVKKVSLYGDYTGVNVA
KQIFANVVELEFYSTSKANSFGFNPLVLGSKTNVINDLFVSKPFTHIDLTQVTLQNSD
NSAIDANKLKQAVGDIYNYRRFERQFQGYEAGGYIDKYLVKNVNTNKDSDDDLVY
RSLKELNLHLEEAYREGDNTYYRVNENYKPGASIYENERASRDSEFQNEILKRAEQN
GVTFDEN
SEQ ID NO: 28: Modified M. genitalium protein M MG48
SLSLNDGSYQSEIDLSGGANFREKFRNFANELSEAITNSPKGLDRPVPKTEISGLIKTG
DNFITPSFKAGYYDHVAEDGSLLSYYQSTEYFNNRVLMPILQTTNGTLMANNRGYD
DVFRQVPRFPGWCNTKPTTVSTSNNLTYDKWTYFACKGSPLYDQYPNHTFEDVKTL
AIDAKDISALKTTIDSEKPTYLIIRGLSGDGSQLNELQLPESVKKVSLYGDYTGVNVA
KQIFANVVELEFYSTSKANSFGFNPLVLGSKTNVINDLFVSKPFTHIDLTQVPLQNSD
NSAIDANKLKQAVGDIYNYRRFERQFQGYFAGGYIDKYLVKNVNTNKDSDDDLVY
RSLKELNLHLEEAYREGDNTYYRVNENYYPGASIYENERASRDSEFQNEILKRAEQN
GVTFDEN
SEQ ID NO: 29: Modified M. genitalium protein M MG49
SLSLNDGSYQSEIDLSGGANFREKFRNFANELSEAITNSPKGLDRPVPKTEISGLIKTG
DNFITPSFKAGYYDHVAEDGSLLSYYQSTEYFNNRVLMPILQTTNGTLMANNRGYD
DVFRQVPRFPGWSNTKATTVSTSNNLTYDKWTYFAAKGSPLYDQYPNHTFEDVKTL
AIDAKDISALKTTIDSEKPTYLIIRGLSGNGSQLNELQLPESVKKVSLYGDYTGVNVA
KQIFANVVELEFYSTSKANSFGFNPLVLGSKTNVINDLFVSKPFTHIDLTQVTLQNSD
NSAIDANKLKQAVGDIYNYRRFERQFQGYFPGGYIDKYLVKNVNTNKDSDDDLVYR
SLKELNLHLEEAYREGDNTYYRVNENYYPGASIYENERASRDSEFQNEILKRAEQNG
VTFDEN
SEQ ID NO: 30: Modified M. pneumoniae protein M MP29
SISLTDGGYRSEIDLGDGSNFREDFRNFANNLSEAITDAPKDLLRPVPKVEVSGLIKTS
STFITPNFKAGYYDQVAEDGKTLKYYQSTEYFNNRVVMPILQTTNGTLTANNRAYD
DIFVDQGVPRFPGWFHDVDKAYYAGSNGQSEYLFKEWNYYVANGSPLYNQYPNHT
FKQIKTIAFDAPRIKQGNTDGINLNLKQRNPDYVIINGLTGDGSTLKDLELPESVKKVS
IYGDYHSINVAKQIFKNVLELEFYSTNQDNNFGFNPLVLGDHTNIIYDLFVSKPFNYID
LTSLELKDNQDNIDASKLKRAVSDIYIRRRFERQMQGYWAGGYIDRYLVKNTNEKN
VNKDNDTVYAALKDINLHLEETYTHGGNTMYRVNENYYPGASAYEAERATRDSEF
QKEIVQR
SEQ ID NO: 31: Modified M. genitalium protein M MG64 (Secretion
Peptide + Fc from IgG1 + 15xGS Linker + MG29dGly1):
MYRMQLLSCIALSLALVTNSAPLEPKSSDKTHTCPPCPAPELLGGPSVFLFPPKPKDTL
YITREPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQG
NVFSCSVMHEALKFHYTQKSLSLSPGAGGGSGGGSGGGSGGGMSLSLNDGSYQSEI
DLSGGANFREKFRNFANELSEAITNSPKGLDRPVPKTEISGLIKTGDNFITPSFKAGYY
DHVAEDGSLLSYYQSTEYFNNRVLMPILQTTDGTLMANNRGYDDVFRQVPRFPGWS
NTKATTVSTSNNLYYDKWTYFAAKGSPLYDQYPNHTFEDVKTLAIDAKDISALKTTI
DSEKPTYLIIRGLSGDGSQLNELQLPESVKKVSLYGDYTGVNVAKQIFANVVELEFYS
TSKANSFGFNPLVLGSKTNVINDLFVSKPFTHIDLTQVTLQNSDNSAIDANKLKQAVG
DIYNYRRFERQFQGYFAGGYIDKYLVKNVNTNKDSDDDLVYRSLKELNLHLEEAYR
EGDNTYYRVNENYYPGASIYENERASRDSEFQNEILKRAEQNGVTFDEN**
SEQ ID NO: 32: Modified M. genitalium protein M MG88 (Secretion
Peptide + Fc from IgG2 + 15xGS Linker + MG29dGly1):
MYRMQLLSCIALSLALVTNSAPLERKSSVECPPCPAPPAAASSVFLFPPKPKDTLMISR
TPEVTCVVVDVSAEDPEVQFNWYVDGVEVHNAKTKPREEQFNSTFRVVSVLTVLHQ
DWLNGKEYKCKVSNKGLPSSIEKTISKTKGQPREPQVYTLPPSREEMTKNQVSLTCL
VKGFYPSDIAVEWESNGQPENNYKTTPPMLDSDGSFFLYSKLTVDKSRWQQGNVFS
CSVMHEALHNHYTQKSLSLSPGAGGGSGGGSGGGSGGGMSLSLNDGSYQSEIDLSG
GANFREKFRNFANELSEAITNSPKGLDRPVPKTEISGLIKTGDNFITPSFKAGYYDHVA
EDGSLLSYYQSTEYFNNRVLMPILQTTDGTLMANNRGYDDVFRQVPRFPGWSNTKA
TTVSTSNNLYYDKWTYFAAKGSPLYDQYPNHTFEDVKTLAIDAKDISALKTTIDSEK
PTYLIIRGLSGDGSQLNELQLPESVKKVSLYGDYTGVNVAKQIFANVVELEFYSTSKA
NSFGFNPLVLGSKINVINDLFVSKPFTHIDLTQVTLQNSDNSAIDANKLKQAVGDIYN
YRRFERQFQGYFAGGYIDKYLVKNVNTNKDSDDDLVYRSLKELNLHLEEAYREGDN
TYYRVNENYYPGASIYENERASRDSEFQNEILKRAEQNGVTFDEN**
SEQ ID NO: 33: Modified M. genitalium protein M MG118 (Protein A
(Z-domain) + 10x GS Linker + MG29):
MKFNKEQQNAFYEILHLPNLNEEQRNAFIQSLKDDPSQSANLLAEAKKLNDAQAPG
GSGGSGGSGSLSLNDGSYQSEIDLSGGANFREKFRNFANELSEAITNSPKGLDRPVPK
TEISGLIKTGDNFITPSFKAGYYDHVAEDGSLLSYYQSTEYFNNRVLMPILQTTNGTL
MANNRGYDDVFRQVPRFPGWSNTKATTVSTSNNLTYDKWTYFAAKGSPLYDQYPN
HTFEDVKTLAIDAKDISALKTTIDSEKPTYLIIRGLSGNGSQLNELQLPESVKKVSLYG
DYTGVNVAKQIFANVVELEFYSTSKANSFGFNPLVLGSKINVINDLFVSKPFTHIDLT
QVTLQNSDNSAIDANKLKQAVGDIYNYRRFERQFQGYFAGGYIDKYLVKNVNTNKD
SDDDLVYRSLKELNLHLEEAYREGDNTYYRVNENYYPGASIYENERASRDSEFQNEI
LKRAEQNGVTFDEN*
SEQ ID NO: 34: Modified M. genitalium protein M MG66 (having the
following mutations: S150E, S196R, S198P, F237T, S232Q, A342V, N274D,
A205P, and T355P):
SLSLNDGSYQSEIDLSGGANFREKFRNFANELSEAITNSPKGLDRPVPKTEISGLIKTG
DNFITPSFKAGYYDHVAEDGSLLSYYQSTEYFNNRVLMPILQTTNGTLMANNRGYD
DVFRQVPRFPGWSNTKPTTVSTSNNLTYDKWTYFAAKGSPLYDQYPNHTFEDVKTL
AIDAKDISALKTTIDSEKPTYLIIRGLSGDGSQLNELQLPESVKKVSLYGDYTGVNVA
KQIFANVVELEFYSTSKANSFGFNPLVLGSKTNVINDLFVSKPFTHIDLTQVPLQNSD
NSAIDANKLKQAVGDIYNYRRFERQFQGYFAGGYIDKYLVKNVNTNKDSDDDLVY
RSLKELNLHLEEAYREGDNTYYRVNENYYPGASIYENERASRDSEFQNEILKRAEQN
GVTFDEN*
SEQ ID NO: 35: Modified M. genitalium protein M MG71 (Protein L B1
Domain + 5xGS Linker + MG29 with an N-terminal deletion of SLSLND):
MEEVTIKANLILPGCCTQTAEFKGTFEKATSEAYAYADTLKKDNGEWTVDVADKGY
TLNIKFAGGSGGSGSYQSEIDLSGGANFREKFRNFANELSEAITNSPKGLDRPVPKTEI
SGLIKTGDNFITPSFKAGYYDHVAEDGSLLSYYQSTEYFNNRVLMPILQTTNGTLMA
NNRGYDDVFRQVPRFPGWSNTKATTVSTSNNLTYDKWTYFAAKGSPLYDQYPNHT
FEDVKTLAIDAKDISALKTTIDSEKPTYLIIRGLSGNGSQLNELQLPESVKKVSLYGDY
TGVNVAKQIFANVVELEFYSTSKANSFGFNPLVLGSKTNVINDLFVSKPFTHIDLTQV
TLQNSDNSAIDANKLKQAVGDIYNYRRFERQFQGYFAGGYIDKYLVKNVNTNKDSD
DDLVYRSLKELNLHLEEAYREGDNTYYRVNENYYPGASIYENERASRDSEFQNEILK
RAEQNGVTFDEN*
SEQ ID NO: 36: Modified M. genitalium protein M MG86 (a dimer containing
GCN4 helix + 15x GS + MG66)
GGCGGMKQLEDKIEELLSKIYHLENEIARLKKLIGEGGGSGGGSGGGSGGGMSLSLN
DGSYQSEIDLSGGANFREKFRNFANELSEAITNSPKGLDRPVPKTEISGLIKTGDNFITP
SFKAGYYDHVAEDGSLLSYYQSTEYFNNRVLMPILQTTNGTLMANNRGYDDVFRQ
VPRFPGWSNTKPTTVSTSNNLTYDKWTYFAAKGSPLYDQYPNHTFEDVKTLAIDAK
DISALKTTIDSEKPTYLIIRGLSGDGSQLNELQLPESVKKVSLYGDYTGVNVAKQIFAN
VVELEFYSTSKANSFGFNPLVLGSKINVINDLFVSKPFTHIDLTQVPLQNSDNSAIDA
NKLKQAVGDIYNYRRFERQFQGYFAGGYIDKYLVKNVNTNKDSDDDLVYRSLKEL
NLHLEEAYREGDNTYYRVNENYYPGASIYENERASRDSEFQNEILKRAEQNGVTFDE
N*
```

1. A modified Mycoplasma protein M or a functional fragment thereof, having one or more amino acid mutations that increase or maintain pH stability or thermostability of the Mycoplasma protein M or a functional fragment thereof relative to wild-type Mycoplasma protein M or a functional fragment thereof, wherein thermostability is maintained up to 75 degrees, 70 degrees, 65 degrees, 60 degrees, 55 degrees, 50 degrees, 45 degrees, 40 degrees, or 38 degrees and/or wherein pH stability is maintained from a pH of 2 to 8, from a pH of 2.5 to 7.5, or from a pH of 4.5 to 7.5.

2. The modified Mycoplasma protein M or a functional fragment thereof of claim 1, having one or more amino acid mutations that increase pH stability or thermostability of the Mycoplasma protein M or a functional fragment relative to wild-type Mycoplasma protein M or a functional fragment thereof.

3. The modified Mycoplasma protein M or a functional fragment thereof of claim 1, wherein the protein M or a functional fragment or derivative thereof is a protein M fragment.

4. The modified Mycoplasma protein M or a functional fragment thereof of claim 1, derived from protein M of Mycoplasma genitalium, Mycoplasma pneumoniae, or Mycoplasma penetrans.

5. The modified Mycoplasma protein M or a functional fragment thereof of claim 1, wherein the protein M functional fragment comprises amino acid residues 17-537, 37-556, 37-482, 37-468, 37-442, 74-468, 74-479, 74-482, 74-468, or 74-442 of M. genitalium protein M (SEQ ID NO:1) or the equivalent residues from another protein M.

6. The modified Mycoplasma protein M or a functional fragment thereof of claim 1, being a fragment from about residue 74 to about residue 479 of M. genitalium protein M (SEQ ID NO:3) or the equivalent residues of M. pneumoniae protein M (SEQ ID NO:23).

7. The modified Mycoplasma protein M or a functional fragment thereof of claim 1, wherein the one or more mutations are at residues 77, 125, 165, 170, and 280 of M. pneumoniae protein M (SEQ ID NO:23) or any combination thereof.

8. The modified Mycoplasma protein M or a functional fragment thereof of claim 7, wherein the one or more mutations are A77E, K125R, V165Q, H170T, and A280V of M. pneumoniae protein M (SEQ ID NO:23) or any combination thereof.

9. The modified Mycoplasma protein M or a functional fragment thereof of claim 1, wherein the one or more mutations are at residues selected from:
a) 159;
b) 182;
c) 102;
d) 161;
e) 86;
f) 101;
g) 147;
h) 236;
i) 348;
j) 424;
k) 147;
l) 321;
m) 389;
n) 442;
o) 119;
p) 179;
q) 186; or
r) 103
of M. genitalium protein M (SEQ ID NO:1) or the equivalent residues of M. pneumoniae protein M (SEQ ID NO:23) or any combination thereof.

10. The modified Mycoplasma protein M or a functional fragment thereof of claim 9, wherein the one or more mutations are selected from:
a) Q159C and A182C;
b) A102C and T161C; or
c) 186C and F101C
of M. genitalium protein M (SEQ ID NO:1) or the equivalent residues of M. pneumoniae protein M (SEQ ID NO:23) or any combination thereof, wherein the modified residues in a), b), or c) are capable of forming a di-sulfide bond between the modified residues (e.g., SEQ ID NO:34).

11. The modified Mycoplasma protein M or a functional fragment thereof of claim 9, wherein the one or more mutations are selected from:
a) H147Y;
b) H236Y;
c) H348Y;
d) H424Y; or
e) H147Q
of M. genitalium protein M (SEQ ID NO:1) or the equivalent residues of M. pneumoniae protein M (SEQ ID NO:23) or any combination thereof, wherein the mutation can prevent histidine protonation at that site at low pH and thereby prevent protein destabilization.

12. The modified Mycoplasma protein M or a functional fragment thereof of claim 9, wherein the one or more mutations are selected from:
a) N321H;
b) Y389H;
c) N442H;
d) P119H;
e) T179H;
f) G186H; or
g) N103H
of M. genitalium protein M (SEQ ID NO:1) or the equivalent residues of M. pneumoniae protein M (SEQ ID NO:23) or any combination thereof, wherein the mutation can increase pH sensitivity thereby improving elution conditions for antibody purification.

13. The modified Mycoplasma protein M or a functional fragment thereof of claim 1, wherein the mutation is at residue 338 of M. genitalium protein M (SEQ ID NO:1) (e.g., Y388N) or the equivalent residues of M. pneumoniae protein M (SEQ ID NO:23).

14. The modified Mycoplasma protein M or a functional fragment thereof of claim 1, wherein the modified Mycoplasma protein M or a functional fragment thereof is further modified to remove one or more glycosylation sites.

15. A modified Mycoplasma protein M or a functional fragment thereof, having a deletion from about residue 185 to about residue 342 of M. genitalium protein M (SEQ ID NO:1) or the equivalent residues of another Mycoplasma protein M.

16-18. (canceled)

19. A fusion protein comprising:
the modified Mycoplasma protein M or a functional fragment thereof of claim 1;
a linker; and
a peptide.

20-39. (canceled)

40. A polynucleotide encoding the modified Mycoplasma protein M or a functional fragment thereof of claim 1.

41-46. (canceled)

47. A vector comprising the polynucleotide of claim 40.

48-49. (canceled)

50. A transformed cell comprising the polynucleotide of claim 40.

51-53. (canceled)

54. A method of inhibiting neutralization of a heterologous agent by neutralizing antibodies upon administration of the heterologous agent to a subject, comprising administering to the subject an effective amount of the Mycoplasma protein M or a functional fragment or derivative thereof of claim 1, thereby inhibiting neutralization of the heterologous agent.

55-105. (canceled)
